(19) United States
(12) Patent Application Publication
(10) Pub. No.: US 2002/0086825 A1
(43) Pub. Date: Jul. 4, 2002

(54) Genes associated with mechanical stress, expression products therefrom, and uses thereof

(76) Inventors: Paz Einat, Ness-Ziona (IL); Orit Segev, Rohovot (IL); Rami Skaliter, Ness-Ziona (IL); Elena Feinstein, Rehovot (IL); Alexander Faerman, Bnei Aiish (IL)

Correspondence Address: Edgar H. Haug, Esq., FROMMER LAWRENCE & HAUG LLP, 745 Fifth Avenue, New York, NY, 10151, US

(21) Appl. No.: 09/802,318

(22) Filed: Mar. 8, 2001

Related U.S. Application Data
(63) Continuation-in-part of application No. 09/729,485, filed on Dec. 4, 2000.
(60) Provisional application No. 60/207,821, filed on May 30, 2000.
(60) Provisional application No. 60/084,944, filed on May 11, 1998.
(60) Provisional application No. 60/085,673, filed on May 15, 1998.

(51) Int. Cl. A61K 39/395; C07K 16/42; A61K 38/17

(52) U.S. Cl. 514/12; 424/146.1; 530/388.26

(57) ABSTRACT

The disclosure relates to mechanical stress induced genes, such as those from human and from mice, probes therefor, tests to identify such genes, expression products of such genes, uses for such genes and expression products, e.g., in diagnosis (for instance risk determination), treatment, prevention, or control, of osteoporosis or factors or processes which lead to osteoporosis; and, to diagnostic, treatment, prevention, or control methods or processes, as well as compositions therefor and methods or processes for making and using such compositions.

### CROSS-REFERENCE TO RELATED APPLICATIONS

This application claims the priority of U.S. patent application Ser. No. 09/729,485 filed Dec. 4, 2000 which claims the priority of U.S. Provisional application Ser. No. 60/207,821, filed May 30, 2000 and U.S. Ser. No. 09/632, 862, filed Aug. 4, 2000.

Reference is also made to U.S. Provisional application Ser. No. 60/084,944, filed May 11, 1998; and the full U.S. utility application, Ser. No. 09/309,862, filed May 11, 1999, and claiming priority from U.S. Provisional application Ser. No. 60/084,944 (herein “the May 14, 1999 Einat et al. full U.S. utility application”); and U.S. Ser. No. 09/312,216, filed, May 14,1999; and U.S. Provisional application Ser. No. 60/085,673, filed May 15, 1998; U.S. Provisional application Ser. No. 60/085,673, filed May 15, 1998; U.S. Provisional application Ser. No. 60/207, 821, filed May 30, 2000; U.S. Ser. No. 09/312,216, filed, May 14, 1999; U.S. Provisional application Ser. No. 60/084,944; and the May 11, 1999 Einat et al. full U.S. utility application; as well as each document or reference cited in these applications, are hereby expressly incorporated herein by reference. Documents or references are also cited in the following text and, each of these documents or references (“herein-cited documents or references”), as well as each document or reference cited in each of the herein-cited documents or references, are hereby expressly incorporated herein by reference. It is explicitly stated that the inventive entity of the May 11, 1999 Einat et al. full U.S. utility application is not another or others as to the inventive entity of this application; and, that the inventive entity of the present application is not another or others as to the inventive entity of the May 11, 1999 Einat et al. full U.S. utility application.

### STATEMENT REGARDING FEDERALLY SPONSORED RESEARCH

Not Applicable.

### FIELD OF THE INVENTION

This invention relates to mechanical stress induced genes, probes therefor, tests to identify such genes, expression products of such genes, uses for such genes and expression products, e.g., in diagnosis (for instance risk determination), treatment, prevention, or control, of osteoporosis or factors or processes which lead to osteoporosis, osteopenia, osteopetrosis, osteoscierosis, osteoarthritis, periodontosis and bone fractures; and, to diagnosis, treatment, prevention, or control methods or processes, as well as compositions therefor and methods or processes for making and using such compositions.

### BACKGROUND OF THE INVENTION

Bone is composed of a collagen-rich organic matrix impregnated with mineral, largely calcium and phosphate. Two major forms of bone exist, compact cortical bone forms the external envelopes of the skeleton and trabecular or medullary bone forms plates that traverse the internal cavities of the skeleton. The responses of these two forms to metabolic influences and their susceptibility to fracture differ.

Bone undergoes continuous remodeling (turnover, renewal) throughout life. Mechanical and electrical forces, hormones and local regulatory factors influence remodeling. Bone is renewed by two opposing activities that are coupled in time and space. Parfitt (1979) Calcif. Tis. Int. 28:1-5. These activities, resorption and formation, are contained within a temporary anatomic structure known as a bone-remodeling unit. Parfitt (1981) Res. Staff Physic. Dec.:60-72. Within a given bone-remodeling unit, old bone is resorbed by osteoclasts. The resorbed cavity created by osteoclasts is subsequently filled with new bone by osteoblasts, synthesizing bone organic matrix.

Peak bone mass is mainly genetically determined, though dietary factors and physical activity can have positive effects. Peak bone mass is attained at the point when skeletal growth ceases, after which time bone loss starts.

In contrast to the positive balance that occurs during growth, in osteoporosis, the resorbed cavity is not completely refilled by bone. Parfitt (1988) in, Osteoporosis: Etiology, Diagnosis, and Management (Riggs and Melton, eds.) Raven Press, New York, pp. 74-93. Osteoporosis, or porous bone, is a progressive and chronic disease characterized by low bone mass and structural deterioration of bone tissue, leading to bone fragility and an increased susceptibility to fractures of the hip, spine, and wrist (diminishing bone strength).

Bone loss occurs without symptoms. The Consensus Development Conference ((1993) Am. J. Med. 94:646-650) defined osteoporosis as “a systemic skeletal disease characterized by low bone mass and microarchitectural deterioration of bone tissue, with a consequent increase in bone fragility and susceptibility to fracture.”

Common types of osteoporosis include postmenopausal osteoporosis; and senile osteoporosis, which generally occurs in later life, e.g., 70+years. See, e.g., U.S. Pat. No. 5,691,153. Osteoporosis is estimated to affect more than 25 million people in the United States (Rosen (1997) Calcif. Tis. Int. 60:225-228); and, at least one estimate asserts that osteoporosis affects 1 in 3 women. Keen et al. (1997) Drugs Aging 11:333-337. Moreover, life expectancy has increased, and in the western world, 17% of women are now over 50 years of age; and, a woman can expect to live one third of her life after menopause. Thus, some estimate that 1 out of every 2 women and 1 out of 5 men will eventually develop osteoporosis; and, that 75 million people in the U.S., Japan and Europe have osteoporosis. The World Summit of Osteoporosis Societies estimates that more than 200 million people worldwide are afflicted with the disease. The actual incidence of the disease is difficult to estimate since the condition is often asymptomatic until a bone fracture occurs. It is believed that there are over 1.5 million osteoporosis-associated bone fractures per year in the U.S. Of these, 300,000 are hip fractures that usually require hospitalization and surgery and may result in lengthy or permanent disability or even death. Spangler et al. “The Genetic Component of Osteoporosis Mini-review”; and http://www.csa.com.osteointro.html).

Osteoporosis is also a major health problem in virtually all societies. Eisman (1996); Wark (1996) Maturitas 23:193-207; and U.S. Pat. No. 5,834,200. There is a 20-30% mortality rate related to hip fractures in elderly women (U.S. Pat. No. 5,691,153); and, such a patient with a hip fracture has a 10-15% greater chance of dying than others of the same age. Further, although men suffer fewer hip injuries than women, men are 25% more likely than women to die within one year of the injury. See Spangler et, al., supra. Also, about 20% of the patients who lived inependently before a hip fracture remain confined in a long-term health care facility one year later. The treatment of osteoporosis and related fractures costs over $10 billion annually.

Osteoporosis treatment helps stop further bone loss and fractures. Common therapeutics include HRT (hormone replacement therapy), bisphosphonates, e.g., alendronate (Fosamax), estrogen and estrogen receptor modulators, progestin, calcitonin, and vitamin D. While there may be numerous factors that determine whether any particular person will develop osteoporosis, a step towards prevention, control or treatment of osteoporosis is determining whether one is at risk for osteoporosis. Genetic factors also play an important role in the pathogenesis of osteoporosis. Ralston (1997); see also Keen et al. (1997); Eisman (1996); Rosen (1997); Cole (1998); Johnston et al. (1995) Bone 17(2 Suppl)19S-22S; Gong et al. (1996) Am. J. Hum. Genet. 59:146-151; and Wasnich (1996) Bone 18(3 Suppl):179S-183S. Some attribute 50-60% of total bone variation (bone mineral density; BMD), depending upon the bone area, to genetic effects. Livshits et al. (1996) Hum. Biol. 68:540-554. However, up to 85%-90% of the variance in bone mineral density may be genetically determined.

Studies have shown from family histories, twin studies, and racial factors, that there may be a predisposition for osteoporosis. Jouanny et al. (1995) Arthritis Rheum. 38:61-67; Garnero et al. (1996) J. Clin. Endrocrinol. Metab. 81:140-146; Cummings (1996) Bone 18(3 Suppl):165S-167S; and Lonzer et al. (1996) Clin. Pediatr. 35:185-189. Several candidate genes may be involved in this, most probably multigenic, process.

Cytokines are powerful regulators of bone resorption and formation under control of estrogen/testosterone, parathyroid hormone and 1,25(OH)2D3. Some cytokines primarily enhance osteoclastic bone resorption e.g. IL-1 (interleukin-1), TNF (tumor necrosis factor) and IL-6 (interleukin-6); while others primarily stimulate bone formation e.g. TGF-β (transforming growth factor-β), IGF (insulin-like growth factor) and PDGF (platelet derived growth factor).

There is need for clinical and epidemiological research for the prevention and treatment of osteoporosis for gaining greater knowledge of factors controlling bone cell activity and regulation of bone mineral and matrix formation and remodeling.

Bone develops via a number of processes. Mesenchymal cells can differentiate directly into bone, as occurs in the flat bones of the craniofacial skeleton; this process is termed intramembranous ossification. Alternatively, cartilage provides a template for bone morphogenesis, as occurs in the majority of human bones. The cartilage template is replaced by bone in a process known as endochondral ossification. Reddi (1981) Collagen Rel. Res. 1:209-226. Bone is also continuously modeled during growth and development and remodeled throughout the life of the organism in response to physical and chemical signals. Development and maintenance of cartilage and bone tissue during embryogenesis and throughout the life-time of vertebrates is very complex. It is widely accepted that a multitude of factors, from systemic hormones to local regulatory factors such as the members of the TGF-p superfamily, cytokines and prostaglandins, act in concert to regulate the continuous processes of bone formation and bone resorption. Disturbance of the balance between osteoblastic bone deposition and osteoclastic bone resorption is responsible for many skeletal diseases.

Diseases of bone loss are a major public health problem especially for women in all Western communities. The most common cause of osteopenia is osteoporosis; other causes include osteomalacia and bone disease related to hyperparathyroidism. Osteopenia has been defined as the appearance of decreased bone mineral content on radiography, but the term more appropriately refers to a phase in the continuum from decreased bone mass to fractures and infirmity.

It is estimated that 30 million Americans are at risk for osteoporosis, the most common among these diseases, and there are probably 100 million people similarly at risk worldwide. Melton (1995) Bone Min. Res. 10:175. These numbers are growing as the elderly population increases. Despite recent successes with drugs that inhibit bone resorption, there is a clear need for specific anabolic agents that will considerably increase bone formation in people who have already suffered substantial bone loss. There are no such drugs currently approved.

Mechanical stimulation induces new bone formation in vivo and increases osteoblastic differentiation and metabolic activity in culture. Mechanotransduction in bone tissue involves several steps: 1) mechanochemical transduction of the signal; 2) cell-to-cell signaling; and 3) increased number and activity of osteoblasts. Cell-to-cell signaling after mechanical stimulus involves prostaglandins, especially those produced by COX-2, and nitric oxide. Prostaglandins induce new bone formation by promoting both proliferation and differentiation of osteoprogenitor cells.

### OBJECTS AND SUMMARY OF THE INVENTION

In a search for agents that enhance osteoblast proliferation/differentiation and bone formation, mechanical force was employed as an osteogenesis inducer and a proprietary gene discovery methodology was carried out to detect genes that are specifically expressed in very early osteo-, chondro-progenitor cells.

The present invention provides human mechanical stress induced genes, expression products of such genes, uses for such genes and expression products for treatment, prevention, control, of osteoporosis or factors or processes which are involved in bone diseases including, but not limited to, osteoporosis, osteopenia, osteopetrosis, osteosclerosis, osteoarthritis, periodontosis and bone fracture. The invention further provides diagnostic, treatment, prevention, control methods or processes as well as compositions.

The invention additionally provides an isolated nucleic acid molecule, and the complement thereof, encoding the protein 608 or a functional portion thereof or a polypeptide, which is at least substantially homologous or identical thereto. The invention encompasses an isolated nucleic acid molecule encoding human protein 608 (or “OCP”) or a functional portion thereof.

The invention further encompasses a method for preventing, treating or controlling osteoporosis or low bone density or other factors associated with, causing or contributing to bone diseases including, but not limited to, osteopenia, osteopetrosis, osteosclerosis, osteoarthritis, periodontosis or symptoms thereof, or other conditions involving mechanical stress or a lack thereof, by administering to a subject in need thereof, a polypeptide or portion thereof provided herein; and accordingly, the invention comprehends uses of polypeptides in preparing a medicament or therapy for such prevention, treatment or control.

The invention also comprehends a method for preventing, treating or controlling osteoporosis or low bone density or other factors causing or contributing to osteoporosis or symptoms thereof or other conditions involving mechanical stress or a lack thereof, by administering a composition comprising a gene or functional portion thereof, an antibody or portion thereof elicited by such an expression product or portion thereof; and, the invention thus further comprehends uses of such genes, expression products, antibodies, portions thereof, in the preparation of a medicament or therapy for such control, prevention or treatment.

The invention further encompasses methods of use of Adlican as described herein for any use of OCP. The Adlican gene, or functional portions thereof, can likewise be used for any purpose described herein for an OCP gene. The invention further encompasses compositions comprising a physiologically acceptable excipient and at least one of Adlican, the Adlican gene and antibodies specific to Adlican.

These and other embodiments are disclosed or are obvious from and encompassed by, the Detailed Description which follows the Brief Description of the Figures below.

### BRIEF DESCRIPTION OF THE FIGURES

The following Detailed Description, given by way of example, but not intended to limit the invention to specific embodiments described, may be understood in conjunction with the accompanying Figures, in which:

FIG. 1 shows the full rat 608 cDNA sequence (SEQ ID NO:1).

FIG. 2 shows the PcDNA3.1-608 construct.

FIG. 3 shows the OCP rat protein amino acid sequence (SEQ ID NO:2).

FIG. 4 shows the results of TNT (transcription—translation) assays.

FIG. 5 shows the structure of Bac 23-261L4.

FIG. 6 shows the structure of Bac 23-241H7.

FIG. 7 shows the sequence analysis of m608p-Lexicon clone (SEQ ID NO:3)—Partial re-sequence. (1) Re-sequenced regions are underlined; (2) Putative exons are in Bold lettering; and (3) ATG-First ATG of coding region (in Italics).

FIG. 8 shows the mouse OCP exon and intron map.

FIG. 9 shows the OCP map of exon-intron borders.

FIG. 10 shows the sequence alignment between genomic human OCP (SEQ ID NO:4) and rat OCP cDNA (SEQ ID NO:5)-2 exons.

FIG. 11 shows the human OCP exon and intron list.

FIG. 12 shows the OCP human cDNA sequence (predicted coding region, SEQ ID NO:6)

FIG. 13 shows the percent identity between A. rat protein/human protein; B. rat protein/mouse protein; C. rat cDNA/human cDNA; and D. rat cDNA/mouse cDNA.

FIG. 14 shows the alignment of rat, human, and mouse OCP cDNA coding regions (rat cDNA: SEQ ID NO:7; human 5+3 corrected: SEQ ID NO:8; and mus cDNA 5: SEQ ID NO:9).

FIG. 15 shows the alignment of rat, human and mouse OCP proteins (rat: SEQ ID NO:10; human 5+3 corrected: SEQ ID NO:11; and mouse 5 corrected: SEQ ID NO:12).

FIG. 16 shows the alignment of rat and human OCP proteins (rat: SEQ ID NO:13; and human 5+3 corrected: SEQ ID NO:14).

FIG. 17 shows the partial mouse OCP protein amino acid sequence (236 aa) (SEQ ID NO:15).

FIG. 18 shows the OCP human protein amino acid sequence (2587 aa) (SEQ ID NO:16).

FIG. 19 shows the OCP protein structure predicted from the OCP gene.

FIG. 20 shows a list of expression patterns of OCP in primary cells and various other cell lines. A. Northern blot of poly A+ RNA RT-PCR from rat primary calvaria cells and MC3T3 cells is shown. The main 8.9kb transcript is present only in calvaria cells. RT-PCR assays with specific OCP primers were performed on total RNA from various lines as indicated on the right side of the figure. In all assays similar amounts of GapDH RT-PCR products were detected in all RNA samples. In addition, B. no GapDH products were detected in any RNA samples, when RT was omitted. (−) represents no expression of OCP, while (+) represents expression. When (− +) are indicated, the expression of OCP is induced only upon specific conditions.

FIG. 21 shows the effects of mechanical stress on MC3T3 pre-osteoblastic cells. RT-PCR for OCP, Cbfal, Osteopontin (OPN) and GAPDH transcripts are as indicated. The results shown are representative of three experiments using total cellular RNA from MC3T3 cells that did not undergo mechanical stress (1), and mechanically stimulated MC3T3 cells (2). The RT-PCR products, were stained with ethidium bromide.

FIG. 22 shows OCP (608) expression in early stages of in vitro osteoblast differentiation from mesenchymal (C3H10T½) and pre-myoblast (C2C12) cells.

FIG. 23 shows that OCP is an early marker of endochondral ossification in P7 rat femoral epiphysis.

FIG. 24 shows that OCP is induced during osteoblastic differentiation of bone marrow stroma cells and is a specific marker of early osteoblastic progenitors in bone marrow.

FIG. 25 shows in vivo regulation of OCP expression in bone marrow formation by various treatments. The results shown are representative of three experiments using total cellular RNA from treated two-month old mice. The different treatments are indicated. The RT-PCR products are marked. Control mice did not undergo any treatment. In each treatment group the left lane represents negative control without the addition of RT, the central lane represents the OCP RT-PCR product and the right lane represents the GapDH RT-PCR product. Bone formation is shown by blood loss, estrogen administration; bon loss, by sciatic neurotomy models.

FIG. 26 shows a low power microphotograph of fractured bone one week after the operation. Note that well-developed woven bone and fibrocartilagenous callus formed at the fracture site. Bone marrow tissue was mainly destroyed by insertion of the wire used for the fracture immobilization. Marked areas are presented at higher magnification in the following figures.

FIG. 27 shows microphotographs of the central part of callus, A. brightfield and B. darkfield. Cells expressing the OCP gene can be seen in the fibrous part of the callus. There was no hybridization signal from chondrocytes.

FIG. 28 shows microphotographs of the callus area marked by 2 in FIG. 26, A. brightfield and B. darkfield. Cells expressing the OCP gene can be seen in a highly vascularized subperiosteal area bordering the cartilagenous part of the callus.

FIG. 29 shows microphotographs of the highly vascularized endiosteal tissue. This was developed in reaction to the wire insertion (area 3 on FIG. 26), A. brightfield and B. darkfield. This tissue contains many cells expressing the OCP gene.

FIG. 30 shows a high power microphotograph of perivascular cells. The perivascular cells express the 608 gene within lacuna of woven bone arrowheads.

FIG. 31 shows a high power microphotograph of periosteum covering the woven bone. Multiple cells display expression of the 608 gene in periosteum. Arrowheads point to two 608 expressing cells within the woven bone.

FIG. 32 shows A. brightfield and B. darkfield microphotographs of a section of fractured bone healed for 4 weeks. Multiple cells in periosteal tissue area of active remodeling of the cancellous bone covering the callus show a hybridization signal.

FIG. 33 shows the boxed area of FIG. 32 presented at higher magnification. Several OCP-expressing cells are concentrated in vascular tissue that fills the cavities resulting from osteoclast activity (marked by astrisks).

FIG. 34 shows in vitro induction of osteoblastic differentiation by transfected OCP.

FIG. 35 shows transient transfections of OCP deletion constructs to calvaria cells. Two OCP deletion constructs (OCP-403,OCP-760) and OCP full length construct were transiently transfected to primary calvaria cells. ALP staining is presented. All deletion constructs show increased osteoblastic colony numbers and colony size compared with transient transfection of the control pCDNA vector.

FIG. 36 shows increased osteoblast differentiation in OCP-transfected ROS cells. RT-PCR assays were with OCP, Cbfal, ALP, BSP and GapDH specific primers as indicated above. The results shown are representative of two experiments using total cellular RNA from: (1) the stable OCP-expressed ROS cell line; and (2) the control ROS cell line (stable transfection with pCDNA). The OCP RT-PCR product is 1020 bp, the Cbfal product is 289 bp, the ALP product is 226 bp, the BSP product is 1048 bp and the GapDH (control) product is 450 bp long. M represents protein markers.

FIG. 37 shows increased osteoblast proliferation in OCP-transfected ROS cells.

FIG. 38 shows OCP induction of bone formation ex vivo. Bigger bones and higher bone mass density were found in bones co-cultivated with OCP transfected cells.

FIG. 39 shows the structure of the Osteocalcin promoter—OCP gene.

FIG. 40 shows autoradiograms of Southern blot analysis of placenta DNAs. “A” shows the results of a Southern blot on the DNA samples from all developed embryos. (Sample 10 is missing due to lack of embryo in the sample). “F,” the injected fragment, served as positive control for the expected size, the arrow marks the expected fragment. “B” shows a section of the autoradiogram of “A” exposed to the sample for additional time. These autoradiograms show that both embryos 20 and 21 are transgenic. “C” shows a repetition of the Southern blot on DNA from three selected embryos, 11, 20 and 21. Embryos 20 and 21 are again detected as transgenic. Embryo 11, which gave an obscured signal on the longer exposure of “A”, is also detected as transgenic in “C.” “F” is genomic DNA from a stable transgenic line produced later. The correct fragment is indicated by an arrow. The more intense fragment found below is a non-specific fragment occasionally observed with the SV40 probe.

FIG. 41 shows A. exogenic OCP expression in transgenic embryos. RT-PCR for exogenic OCP transcripts was performed. The results are representative of three experiments using total cellular RNA from embryo tails. The RT-PCR products that are marked were visualized by staining with ethidium bromide. B. GapDH primers were used to show that differences in OCP transcript abundance did not reflect variation in the efficiency of the RT reaction.

FIG. 42 shows the characterization of osteocalcin promoter of OCP transgenic embryos (E17 embryos). Calvaria, tibia and femur lengths were measured in μm. All measurements include only the calcified regions stained by Alizarin Red. A. shows calvaria length/width, B. shows calvaria length/width (%).

FIG. 43 shows Alizarin Red staining of OC-OCP transgenic embryo long bones showing that OCP induces bone formation in vivo. Cells shown are osteoblasts, chondrocytes and liver/bloodstream.

FIG. 44 shows Alizarin Red staining of calvaria bones from transgenic and control embryos. Higher calcification (represented by Alizarin Red staining) was detected when transgenic embryo calvaria bones were stained in comparison with their littermates. The transgenic embryo calvaria bones were longer and wider.

FIG. 45 compares clone 14C10 to the Lexicon clone.

FIG. 46 shows pMCSIEm608prm5.5.

FIG. 47 shows the sequence of the mouse OCP promoter region (proximal 5.5 kb fragment) (SEQ ID NO:17) cloned into pMCSIE/pGL3-basic.

FIG. 48 shows the sequence of the 5′ end of clone p14C10 (SEQ ID NO:18) encoding the mouse OCP promoter region.

FIG. 49 shows the proximal regulatory region of human and mouse OCP genes.

FIG. 50 shows the sequences of the primer (SEQ ID NO:19) and QB3 (CMF608) (SEQ ID NO:20).

FIG. 51 shows the Adlican and amino acid sequence (SEQ ID NO: 21).

FIG. 52 shows the Adlican mRNA sequence (SEQ ID NO: 22)

### DETAILED DESCRIPTION OF THE INVENTION

The present invention is related to the discovery of a novel gene, CMF608 (“OCP”), the expression of which is upregulated by mechanical stress on primary calvaria cells. Several functional features identify OCP as the most specific early marker of osteo- or chondro-progenitor cells as well as an inducer of osteoblast proliferation and differentiation As used herein, the same gene of the invention may be referred to either as “608” or “OCP.” RNA refers to RNA isolated from cell cultures, cultured tissues or cells or tissues isolated from organisms which are stimulated, differentiated, exposed to a chemical compound, infected with a pathogen, or otherwise stimulated. As used herein, translation is defined as the synthesis of protein encoded by an mRNA template.

As used herein, stimulation of translation, transcription, stability or transportation of unknown target mRNA or stimulating element, includes chemically, pathogenically, physically, or otherwise inducing or repressing an mRNA population encoded by genes derived from native tissues and/or cells under pathological and/or stress conditions. In other words, stimulating the expression of an mRNA with a stress inducing element or “stressor” includes, but is not limited to, the application of an external cue, stimulus, or stimuli that stimulates or initiates translation of an mRNA stored as untranslated mRNA in the cells from the sample. The stressor may cause an increase in stability of certain mRNAs, or induce the transport of specific mRNAs from the nucleus to the cytoplasm. The stressor may also induce specific gene transcription. In addition to stimulating translation of mRNA from genes in native cells/tissues, stimulation can include induction and/or repression of genes under pathological and/or stress conditions. The method utilizes a stimulus or stressor to identify unknown target genes regulated at the various possible levels by the stress inducing element or stressor.

More in particular, with respect to nucleic acid molecules (rat 608 and human 608 genes) and polypeptides expressed from them, the invention further comprehends isolated and/or purified nucleic acid molecules and isolated and/or purified polypeptides having at least about 70%, preferably at least about 75% or about 77% identity or homology (“substantially homologous or identical”); advantageously at least about 80% or about 83%, such as at least about 85% or about 87% homology or identity (“significantly homologous or identical”); for instance at least about 90% or about 93% identity or homology (“highly homologous or identical”); more advantageously at least about 95%, e.g., at least about 97%, about 98%, about 99% or even about 100% identity or homology (“very highly homologous or identical” to “identical”); or from about 84-100% identity considered (“highly conserved”). The invention also comprehends that these nucleic acid molecules and polypeptides can be used in the same fashion as the herein or aforementioned nucleic acid molecules and polypeptides.

Nucleotide sequence homology can be determined using the “Align” program of Myers and Miller, ((1988) CABIOS 4:11-17) and available at NCBI. Alternatively or additionally, the term “homology” or “identity,” for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, AGTCAGTC has a sequence similarity of 75% to AATCAATC (N_{ref}=8; N_{dif}=2).

Alternatively or additionally, “homology” or “identity” with respect to sequences can refer to the number of positions with identical nucleotides or amino acid residues divided by the number of nucleotides or amino acid residues in the shorter of the two sequences wherein alignment of the two sequences can be determined in accordance with the Wilbur and Lipman algorithm ((1983) Proc. Natl. Acad. Sci. USA 80:726), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc., CA). When RNA sequences are said to be similar, or have a degree of sequence identity or homology with DNA sequences, thymidine (T) in the DNA sequence is considered equal to uracil (U) in the RNA sequence (see also alignment used in the Figures). RNA sequences within the scope of the invention can be derived from DNA sequences or their complements, by substituting thymidine (T) in the DNA sequence with uracil (U).

Additionally or alternatively, amino acid sequence similarity or identity or homology can be determined, for instance, using the BlastP program (Altschul et al. Nucl. Acids Res. 25:3389-3402) and available at NCBI. The following references provide algorithms for comparing the relative identity or homology of amino acid residues of two proteins, and additionally, or alternatively, with respect to the foregoing, the teachings in these references can be used for determining percent homology or identity. Smith et al. (1981) Adv. Appl. Math. 2:482-489; Smith et al. (1983) Nucl. Acids Res. 11:2205-2220; Devereux et al. (1984) Nucl. Acids Res. 12:387-395; Feng et al. (1987) J. Molec. Evol. 25:351-360; Higgins et al. (1989) CABIOS 5:151-153; and Thompson et al. (1994) Nucl. Acids Res. 22:4673480.

As to uses, the inventive genes and expression products as well as genes identified by the herein disclosed methods and expression products thereof and the compositions comprising Adlican or the Adlican gene (including “functional” variations of such expression products, and truncated portions of herein defined genes such as portions of herein defined genes which encode a functional portion of an expression product) are useful in treating, preventing or controlling or diagnosing mechanical stress conditions or absence or reduced mechanical stress conditions.

As described herein, Adlican, including functional portions thereof, can be used in all methods suitable for OCP. The sequence homology between Adlican and human OCP provides this novel use of the Adlican protein. Adlican is provided, for instance, in AF245505.1:1.8487. Adlican is named for “ADhesion protein with Leucine-rich repeats has immunoglobulin domains related to perleCAN”; and shows elevated expression in cartilage from osteoarthritis patients. The Adlican gene, or functional portions thereof, can likewise be used for any purpose described herein for an OCP gene. The invention further encompasses compositions comprising a physiologically acceptable excipient and at least one of Adlican, the Adlican gene and antibodies specific to Adlican.

For instance, OCP expression is related to proliferation and differentation of osteoblasts and chondrocytes. The expression product of OCP, or cells or vectors expressing OCP may cause cells to selectively proliferate and differentiate and thereby increase or alter bone density. Detecting levels of OCP mRNA or expression and comparing it to “normal” non-osteopathic levels may allow one to detect subjects at risk for osteoporosis or lower levels of osteoblasts and chondrocytes.

The medicament or treatment can be any conventional medicament or treatment for osteoporosis. Alternatively, or additionally, the medicament or treatment can be the particular protein of the gene detected in the inventive methods, or that which inhibits that protein, e.g., binds to it. Similarly, additionally, or alternatively, the medicament or treatment can be a vector which expresses the protein of the gene detected in the inventive methods or that which inhibits expression of that gene; again, for instance, that which can bind to it and/or otherwise prevents its transcription or translation. The selection of administering a protein or that which expresses it, or of administering that which inhibits the protein or the gene expression, can be done without undue experimentation, e.g., based on down-regulation or up-regulation as determined by inventive methods (e.g., in the osteoporosis model).

In the practice of the invention, one can employ general methods in molecular biology. Standard molecular biology techniques known in the art and not specifically described are generally followed as in Sambrook et al. (1989, 1992) Molecular Cloning: A Laboratory Manual, Cold Spring Harbor Laboratory, New York; and Ausubel et al. (1989) Current Protocols in Molecular Biology, John Wiley and Sons, Baltimore, Md.

PCR comprising the methods of the invention is performed in a reaction mixture comprising an amount, typically between <10 ng-200 ng template nucleic acid; 50-100 p moles each oligonucleotide primer; 1-1.25 mM each deoxynucleotide triphosphate; a buffer solution appropriate for the polymerase used to catalyze the amplification reaction; and 0.5-2 Units of a polymerase, most preferably a thermostable polymerase (e.g., Taq polymerase or Tth polymerase).

Antibodies may be used in various aspects of the invention, e.g., in detection or treatment or prevention methods. Antibodies can be monoclonal, polyclonal or recombinant for use in the immunoassays or other methods of analysis necessary for the practice of the invention. Conveniently, the antibodies may be prepared against the immunogen or antigenic portion thereof for example a synthetic peptide based on the sequence, or prepared recombinantly by cloning techniques or the natural gene product and/or portions thereof may be isolated and used as the immunogen. The genes are identified as set forth in the present invention and the gene product identified. Immunogens can be used to produce antibodies by standard antibody production technology well known to those skilled in the art as described generally in Harlow and Lane (1988) Antibodies: A Laboratory Manual, Cold Spring Harbor Laboratory, Cold Spring Harbor, N.Y.; and Borrebaeck (1992) Antibody Engineering—A Practical Guide, W.H. Freeman and Co. Antibody fragments can also be prepared from the antibodies and include Fab, F(ab′)2, Fv and scFv prepared by methods known to those skilled in the art. Bird et al. (1988) Science 242:423-426. Any peptide having sufficient flexibility and length can be used as an scFv linker. Usually the linker is selected to have little to no immunogenicity. An example of a linking peptide is (GGGGS)₃, which bridges approximately 3.5 nm between the C-terminus of one V region and the N-terminus of another V region. Other linker sequences can also be used, and can provide additional functions, such as a means for attaching a drug or a solid support.

For producing polyclonal antibodies, a host, such as a rabbit or goat, is immunized with the immunogen or an immunogenic fragment thereof, generally with an adjuvant and, if necessary, coupled to a carrier; and antibodies to the immunogen are collected from the sera of the immunized animal. The sera can be adsorbed against related immunogens so that no cross-reactive antibodies remain in the sera rendering the polyclonal antibody monospecific.

For producing monoclonal antibodies (mAbs), an appropriate donor, generally a mouse, is hyperimmunized with the immunogen and splenic antibody producing cells are isolated. These cells are fused to an immortal cell, such as a myeloma cell, to provide an immortal fused cell hybrid that secretes the antibody. The cells are then cultured, in bulk, and the mAbs are harvested from the culture media for use. Hybridoma cell lines provide a constant, inexpensive source of chemically identical antibodies and preparations of such antibodies can be easily standardized. Methods for producing mAbs are well known to those of ordinary skill in the art. See, e.g. U.S. Pat. No. 4,196,265.

For producing recombinant antibodies, mRNAs from antibody producing B lymphocytes of animals, or hybridomas are reverse-transcribed to obtain cDNAs. See generally, Huston et al. (1991) Met. Enzymol. 203:46-88; Johnson and Bird (1991) Met. Enzymol. 203:88-99; and Mernaugh and Mernaugh (1995) In, Molecular Methods in Plant Pathology (Singh and Singh eds.) CRC Press Inc. Boca Raton, Fla., pp. 359-365). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

Antibodies can be bound to a solid support substrate or conjugated with a detectable moiety or be both bound and conjugated as is well known in the art. For a general discussion of conjugation of fluorescent or enzymatic moieties see, Johnston and Thorpe (1982) Immunochemistry in Practice, Blackwell Scientific Publications, Oxford. The binding of antibodies to a solid support substrate is also well known in the art. See for a general discussion, Harlow and Lane (1988); and Borrebaeck (1992). The detectable moieties contemplated with the present invention include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹³C and iodination.

Antibodies can also be used as an active agent in a therapeutic composition and such antibodies can be humanized, for instance, to enhance their effects. See, Huls et al. Nature Biotech. 17:1999. “Humanized” antibodies are antibodies in which at least part of the sequence has been altered from its initial form to render it more like human immunoglobulins. In one version, the H chain and L chain C regions are replaced with human sequence. In another version, the CDR regions comprise amino acid sequences from the antibody of interest, while the V framework regions have also been converted human sequences. See, for example, EP 0329400. In a third version, V regions are humanized by designing consensus sequences of human and mouse V regions, and converting residues outside the CDRs that are different between the consensus sequences. The invention encompasses humanized mAbs.

The expression product from the gene or portions thereof can be useful for generating antibodies such as monoclonal or polyclonal antibodies which are useful for diagnostic purposes or to block activity of expression products or portions thereof or of genes or a portion thereof, e.g., as therapeutics.

The genes of the present invention or portions thereof, e.g., a portion thereof which expresses a protein which function the same as or analogously to the full length protein, or genes identified by the methods herein can be expressed recombinantly, e.g., in Escherichia coli or in another vector or plasmid for either in vivo expression or in vitro expression. The methods for making and/or administering a vector or recombinant or plasmid for expression of gene products of genes of the invention or identified by the invention or a portion thereof either in vivo or in vitro can be any desired method, e.g., a method which is by or analogous to the methods disclosed in: U.S. Pat. Nos. 4,603,112; 4,769,330; 5,174,993; 5,505,941; 5,338,683; 5,494,807; 4,394,448; 4,722,848; 4,745,051; 4,769,331; 5,591,639; 5,589,466; 4,945,050; 5,677,178; 5,591,439; 5,552,143; and 5,580,859; U.S. patent application Ser. No. 920,197, filed Oct. 16, 1986; WO 94/16716; WO 96/39491; WO91/11525; WO 98/33510; WO 90/01543; EP 0 370 573; EP 265785; Paoletti (1996) Proc. Natl. Acad. Sci. USA 93:11349-11353; Moss (1996) Proc. Natl. Acad. Sci. USA 93:11341-11348; Richardson (Ed) (1995) Methods in Molecular Biology 39, “Baculovirus Expression Protocols,” Humana Press Inc.; Smith et al. (1983) Mol. Cell. Biol. 3:2156-2165; Pennock et al. (1984) Mol. Cell. Biol. 4:399-406; Roizman Proc. Natl. Acad. Sci. USA 93:11307-11312; Andreansky et al. Proc. Natl. Acad. Sci. USA 93:11313-11318; Robertson et al. Proc. Natl. Acad. Sci. USA 93:11334-11340; Frolov et al. Proc. Natl. Acad. Sci. USA 93:11371-11377; Kitson et al. (1991) J. Virol. 65:3068-3075; Grunhaus et al. (1992) Sem. Virol. 3:237-52; Ballay et al. (1993) EMBO J. 4:3861-65; Graham (1990) Tibtech 8:85-87; Prevec et al. J. Gen. Virol. 70:429-434; Felgner et al. (1994) J. Biol. Chem. 269:2550-2561; (1993) Science 259:1745-49; McClements et al. (1996) Proc. Natl. Acad. Sci. USA 93:11414-11420; Ju et al. (1998) Diabetologia 41:736-739; and Robinson et al. (1997) Sem. Immunol. 9:271-283.

The expression product generated by vectors or recombinants can also be isolated and/or purified from infected or transfected cells; for instance, to prepare compositions for administration to patients. However, in certain instances, it may be advantageous to not isolate and/or purify an expression product from a cell; for instance, when the cell or portions thereof enhance the effect of the polypeptide.

As used herein, “treatment” refers to clinical intervention in an attempt to alter the natural course of the individual or cell being treated, and may be performed either for prophylaxis or during the course of clinical pathology. Desirable effects of the treatment include preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis.

An inventive vector or recombinant expressing a gene or a portion thereof identified herein or from a method herein can be administered in any suitable amount to achieve expression at a suitable dosage level, e.g., a dosage level analogous to the herein mentioned dosage levels (wherein the gene product is directly present). The inventive vector or recombinant nucleotide can be administered to a patient or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu. In plasmid compositions, the dosage should be a sufficient amount of plasmid to elicit a response analogous to compositions wherein gene product or a portion thereof is directly present; or to have expression analogous to dosages in such compositions; or to have expression analogous to expression obtained in vivo by recombinant compositions. For instance, suitable quantities of plasmid DNA in plasmid compositions can be 1 μg to 100 mg, preferably 0.1 to 10 mg, e.g., 500 μg, but lower levels such as 0.1 to 2 mg or preferably 1-10 μg may be employed. Documents cited herein regarding DNA plasmid vectors can be consulted for the skilled artisan to ascertain other suitable dosages for DNA plasmid vector compositions of the invention, without undue experimentation.

Compositions for administering vectors can be as in or analogous to such compositions in documents cited herein or as in or analogous to compositions herein described, e.g., pharmaceutical or therapeutic compositions and the like.

Thus, the invention comprehends in vivo gene expression which is sometimes termed “gene therapy.” Gene therapy can refer to the transfer of genetic material (e.g. DNA or RNA) of interest into a host subject or patient to treat or prevent a genetic or acquired disease, condition or phenotype. The particular gene that is to be used or which has been identified as the target gene is identified as set forth herein. The genetic material of interest encodes a product (e.g. a protein, polypeptide, peptide or functional RNA) the production in vivo of which is desired. For example, the genetic material of interest can encode a hormone, receptor, enzyme, polypeptide or peptide of therapeutic value. For a review see, in general, the text “Gene Therapy” (Advances in Pharmacology 40, Academic Press, 1997).

Two basic approaches to gene therapy have evolved: (1) ex vivo; and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, homologous recombination, etc.) and, an expression system as needed and then the modified cells are expanded in culture and returned to the host/patient. These genetically reimplanted cells have been shown to produce the transfected gene product in situ. In in vivo gene therapy, target cells are not removed from the subject; rather, the gene to be transferred is introduced into the cells of the recipient organism in situ, that is within the recipient. Alternatively, if the host gene is defective, the gene is repaired in situ. Culver (1998) Antisense DNA & RNA Based Therapeutics, February, 1998, Coronado, Calif. These genetically altered cells have been shown to produce the transfected gene product in situ.

The gene expression vehicle is capable of delivery/transfer of heterologous nucleic acid into a host cell. The expression vehicle may include elements to control targeting, expression and transcription of the nucleic acid in a cell-selective manner as is known in the art. It should be noted that often the 5′UTR and/or 3′UTR of the gene may be replaced by the 5′ UTR and/or 3′UTR of the expression vehicle. Therefore, as used herein, the expression vehicle may, as needed, not include the 5′UTR and/or 3′UTR shown in sequences herein and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any non-translated DNA sequence that works contiguously with the coding sequence (in cis) to change the basal transcription level dictated by the promoter. The expression vehicle can also include a selection gene as described herein.

Vectors can be introduced into cells or tissues by any one of a variety of known methods within the art. Such methods can be found generally described in Sambrook et al. (1989, 1992); Ausubel et al. (1989); Chang et al. (1995) Somatic Gene Therapy, CRC Press, Ann Arbor, Mich.; Vega et al. (1995) Gene Targeting, CRC Press, Ann Arbor, Mich.; Vectors: A Survey of Molecular Cloning Vectors and Their Uses, Butterworths, Boston Mass. (1988); and Gilboa et al. (1986) BioTech. 4:504-512, as well as other documents cited herein and include, for example, stable or transient transfection, lipofection, electroporation and infection with recombinant viral vectors. In addition, see U.S. Pat. No. 4,866,042 for vectors involving the central nervous system; and also U.S. Pat. Nos. 5,464,764 and 5,487,992 for positive-negative selection methods.

Introduction of nucleic acids by infection offers advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed cell culture. Viral vectors can also be modified with specific receptors or ligands to alter target specificity through receptor-mediated events.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur. Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Delivery of gene products (products from herein defined genes: genes identified herein or by inventive methods or portions thereof) and/or antibodies or portions thereof and/or agonists or antagonists (collectively or individually “therapeutics”), and compositions comprising the same, as well as of compositions comprising a vector expressing gene products, can be done without undue experimentation from this disclosure and the knowledge in the art.

The pharmaceutically “effective amount” for purposes herein is thus determined by such considerations as are known in the art. The amount must be effective to achieve improvement including but not limited to improved survival rate or more rapid recovery, or improvement or amelioration or elimination of symptoms and other indicators, e.g., of osteoporosis, for instance, improvement in bone density, as are selected as appropriate measures by those skilled in the art.

It is noted that humans are treated generally longer than the mice or other experimental animals exemplified herein. Human treatment has a length proportional to the length of the disease process and drug effectiveness. The doses may be single doses or multiple doses over a period of several days, but single doses are preferred. Thus, one can scale up from animal experiments, e.g., rats, mice, and the like, to humans, by techniques from this disclosure and the knowledge in the art, without undue experimentation.

The present invention provides an isolated nucleic acid molecule containing nucleotides having a sequence set forth in at least one of SEQ ID NO:1, SEQ ID NO:3, SEQ ID NO:6 or SEQ ID NO:20, supplements thereof and a polynucleotide having a sequence that differs from SEQ ID NO:1, SEQ ID NO:3, SEQ ID NO:6 or SEQ ID NO:20 due to the degeneracy of the genetic code or a functional portion thereof or a polynucleotide which is at least substantially homologous or identical thereto. In a prefered embodiment, the nucleic acid molecule comprises a polynucleotide having at least 15 nucleotides from SEQ ID NO: 1, SEQ ID NO:3, SEQ ID NO:6 or SEQ ID NO:20, prefereably at least 50 nucleotides and more preferably at least 100 nucleotides.

The present invention also provides a composition of the isolated nucleic acid molecule, a vector comprising the isolated nucleic acid molecule, a composition containing said vector and a method for preventing, treating or controlling bone diseases including, but not limited to, osteoporosis, osteopenia, osteopetrosis, osteosclerosis, osteoarthritis, periodontosis, bone fractures or low bone density or or other conditions involving mechanical stress or a lack thereof in a subject, comprising administering the inventive composition, or the inventive vector, and a method for preparing a polypeptide comprising expressing the isolated nucleic acid molecule or comprising expressing the polypeptide from the vector.

The present invention further provides a method for preventing, treating or controlling osteoporosis, osteopenia, osteopetrosis, osteosclerosis, osteoarthritis, periodontosis, bone fractures or low bone density or other factors causing or contributing to osteoporosis or symptoms thereof or other conditions involving mechanical stress or a lack thereof in a subject, comprising administering an isolated nucleic acid molecule or functional portion thereof or a polypeptide comprising an expression product of the gene or functional portion of the polypeptide or an antibody to the polypeptide or a functional portion of the antibody. In one embodiment of the invention, the isolated nucleic acid molecule encodes a 10 kD to 30 kD N-terminal cleavage product of the OCP protein. Preferably, the N-terminal cleavage product comprises of a polypeptide of about 25 kD.

The present invention provides an isolated polypeptide encoded by the inventive polynucleotide. In one embodiment of the invention, the polypeptide is identified as human OCP or a functional portion thereof or a polypeptide which is at least substantially homologous or identical thereto. Preferably, the functional portion comprises a N-terminal polypeptide having a molecular weight of 10 kD to 30 kD. More preferably, the the functional portion comprises an N-terminal polypeptide having a molecular weight of about 25 kD.

The present invention also provides a composition comprising one or of isolated polypeptides, an antibody specific for the polypeptide or a functional portion thereof, a composition comprising the antibody or a functional portion thereof, and a method for treating or preventing osteoporosis, or fracture healing, bone elongation, or periodontosis in a subject, comprising administering to the subject a N-terminal polypeptide having a molecular weight of between 10 kD and 30 kD, preferably about 25 kD.

The present invention provides for a method of treating or preventing osteoarthritis, osteopetrosis, or osteosclerosis, comprising administering to a subject an effective amount of a chemical or a neutralizing mAbs that inhibit the activity of the N-terminal polypeptide having a molecular weight of between 10 kD and 30 kD, preferably about 25 kD.

As used herein, the term “subject,” “patient,” “host” include, but are not limited to human, bovine, pig, mouse, rat, goat, sheep and horse.

Those skilled in the art will recognize that the components of the compositions should be selected to be chemically inert with respect to the gene product and optional adjuvant or additive. This will present no problem to those skilled in chemical and pharmaceutical principles, or problems can be readily avoided by reference to standard texts or by simple experiments (not involving undue experimentation), from this disclosure and the documents cited herein.

A better understanding of the present invention and of its many advantages will be had from the following examples, given by way of illustration and as a further description of the invention.

### Experimental Details

TGF-β1 is known as a principal inducer of connective tissue growth factor (CTGF, cef10, fisp12, cyr61, βIG-M1, βIG-M2, non-protooncogene) expression. The latter contains four distinct structural modules, each of them homologous to distinct domains in other extracellular proteins such as von Willebrand factor, slit, thrombospondins, fibrillar collagens, IGF-binding proteins and mucins. CTGF expression is induced not only by TGF-β1, but also by BMP2 (bone morphogenic factor 2) and during wound repair. In embryogenesis, its expression is found in developing cartilaginous elements, including limbs, ribs, prevertebrae, chondrocranium and craniofascial elements (Meckel's cartilage). Thus, CTGF transcription correlates with differentiation of chondrocytes of both mesodermal and ectodermal origin. In culture, CTGF is expressed in chondrocytes but not in osteoblasts. A possible role for CTGF in endochondral ossification is suspected because of its responsiveness to BMP2. In fibroblasts, CTGF expression causes upregulation of α-1-collagen, α-5-integrin and fibronectin.

### EXAMPLE 1

### CMF608 Gene Expression by in Situ Hybridization

The CMF608 gene expression pattern was studied by in situ hybridization on sections of bones from ovariectomized and sham-operated rats. Female Wistar rats weighing 300-350 g were subjected to ovariectomy under general anesthesia. Control rats were operated on in the same way but ovaries were not excised—sham operation.

Three weeks after the operation, rats were sacrificed and tibia were excised together with the knee joint. Bones were fixed for three days in 4% paraformaldehyde and then decalcified for four days in a solution containing 5% formic acid and 10% fermalin. Decalcified bones were postfixed in 10% formalin for three days and embedded into paraffin.

The ectopic bone formation model was employed to study the bone development CMF608 gene expression pattern. Rat bone marrow cells were seeded into cylinders of demineralized bone matrix prepared from rat tibiae. Cylinders were implanted subcutaneously into adult rats. After three weeks, rats were sacrificed and implants were decalcified and embedded into paraffin as described above for tibial bones.

The 6 μm sections were prepared and hybridized in situ. After hybridization, sections were dipped into nuclear track emulsion and exposed for three weeks at 4° C. Autoradiographs were developed, stained with hematoxylin-eosin and studied under microscopy using brightfield and darkfield illumination.

For further assessment of cell and tissue specificity of CMF608 gene expression, an in situ hybridization study was performed on sections of multitissue block containing multiple samples of adult rat tissues. The CMF608 expression developmental pattern was studied on sagittal sections of mouse embryos of 12.5, 14.5 and 16.5 days postconception (dpc) stages.

Microscopic study of hybridized sections of long bones revealed a peculiar pattern of CMF608 probe hybridization. The hybridization signal can be seen mainly in fibroblast-like cells found in several locations throughout the sections. Prominent accumulations of these cells can be seen in the area of periosteal modeling in metaphysis, and also in regions of active remodeling of compact bone in diaphysis: at the boundary between bone marrow and endosteal osteoblasts and in periosteum; also in close contact with osteoblasts. Perivascular connective tissue filling Volkmann's canals in compact bone in diaphysis and epiphysis also contains CMF608-expressing cells. No hybridization was found within cancellous bone and in bone marrow. This hybridization pattern suggests that cells expressing CMF608 are associated with areas of remodeling of preexisting bone and are not involved in primary endochondral ossification.

At the growth plate level, CMF608 expressing cells can be seen in the perichondral fibrous ring of LaCroix. Some investigators regard this fibrous tissue as the aggregation of residual mesenchymal cells able to differentiate into both osteoblasts and chondrocytes. In this respect it is noteworthy that single cells expressing CMF608 can be seen in epiphyseal cartilage. These CMF608-expressing cells are rounded cells within the lateral segment of epiphysis (sometimes in close vicinity to the LaCroix ring) and flattened cells covering the articulate surface. Most cells in articulate cartilage and all chondrocytes on the growth plate do not show CMF608 expression. Ovariectomy did not alter the intensity and pattern of CMF608 expression in bone tissue.

In ectopic bone sections, CMF608 hybridization signal can be seen in some fibroblast-like cells either scattered within unmineralized connective tissue matrix or concentrated at the boundary between this tissue and osteoblasts of immature bone.

CMF608 gene expression patterns revealed by in situ hybridization in bone and cartilage indicate that its expression marks some skeletal tissue elements able to differentiate into two skeletal cell types—osteoblasts and chondrocytes. The terminal differentiation of these cells appears to be accompanied by down-regulation of CMF608 expression. The latter observation is supported by the peculiar temporal pattern of CMF608 expression in primary cultures of osteogenic cells isolated from calvaria bones of rat fetuses. In these cultures, expression was revealed by in situ hybridization in the vast majority of cells after one and two weeks of incubation in vitro. Three and four week old cultures showing signs of ossification contained no CMF608 expressing cells. Significantly, no hybridization signal was found on sections of multitissue block hybridized to CMF608 probe suggesting high specificity of this gene expression for the skeletal tissue in adult organisms.

In situ hybridization study of embryonic sections demonstrated that at 12.5 dpc weak hybridization signal can be discerned in some mesenchymal cells in several locations throughout the embryonic body. The most prominent signal is found in the head in loose mesenchymal tissue surrounding the olfactory epithelium and underlying the surface epithelium of nose tip. Other mesenchymal cells in the head also show hybridization signal: non-cartilaginous part of basisphenoid bone primordium and mesenchyme surrounding the dental laminae (tooth primordia) in the mandible.

In the trunk, expression can be detected in less developed vertebrae primordia in the thoraco-lumbar region. The hybridization signal here marks the condensed portion of sclerotomes. Another area of the trunk showing hybridization signal is comprised of a thin layer of mesenchymal cells in the anterior part of thoracic body wall.

At later stages of development, 14.5 and 16.5 dpc, probe CMF608 gave no hybridization signal. Thus, it appears that during embryonic development the CMF608 gene is transiently expressed by at least some mesenchymal and skeleton-forming cells. This expression is down-regulated at later stages of development. More detailed study of late embryonic and postnatal stages of development reveals the timing of appearance of CMF608 expressing cells in bone tissue.

### EXAMPLE 2

### Isolation of Rat OCP

Primary rat calvaria cells grown on elastic membranes that were stretched for 20 minutes provided a model system for a stimulator of bone formation following mechanical force. Gene expression patterns were compared before and after the application of mechanical force.

OCP expression was upregulated approximately 3-fold by mechanical force. This was detected both by microarray analysis and by Northern blot analysis using poly (A)+ RNA from rat calvaria cells before and after the mechanical stress. In rat calvaria primary cells and in rat bone extract this gene was expressed as a main RNA species of approximately 8.9 kb and a minor RNA transcript of approximately 9 kb. The hybridization signal was not detected in any other rat RNA from various tissue sources, including testis, colon, intestine, kidney, stomach, thymus, lung, uterus, heart, brain, liver, eye, and lymph node.

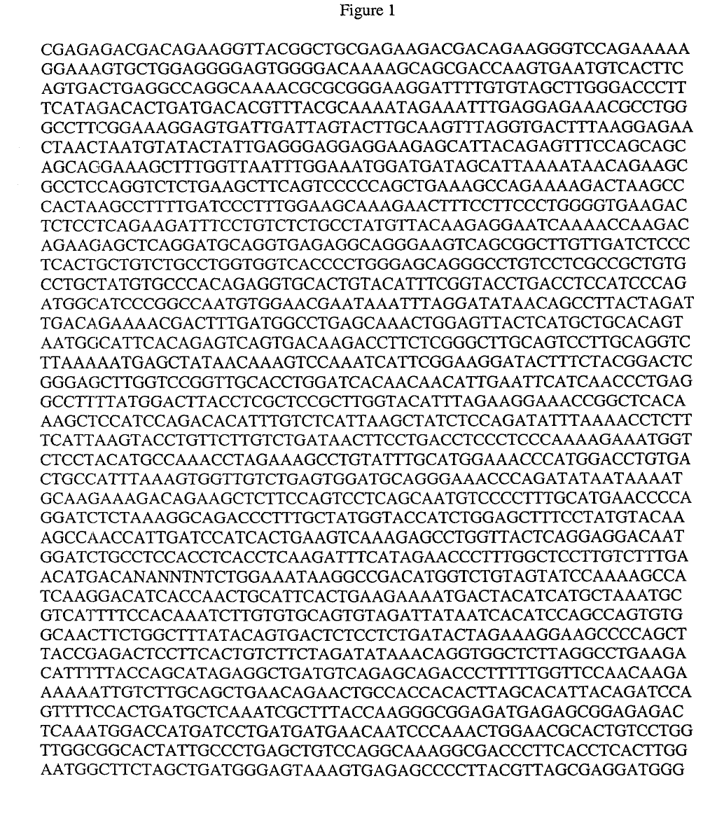

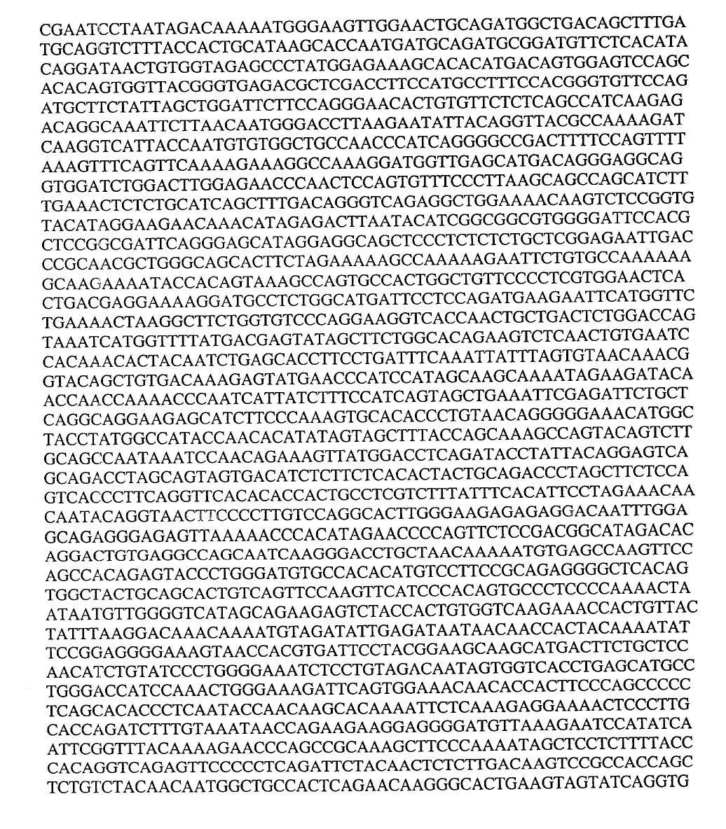

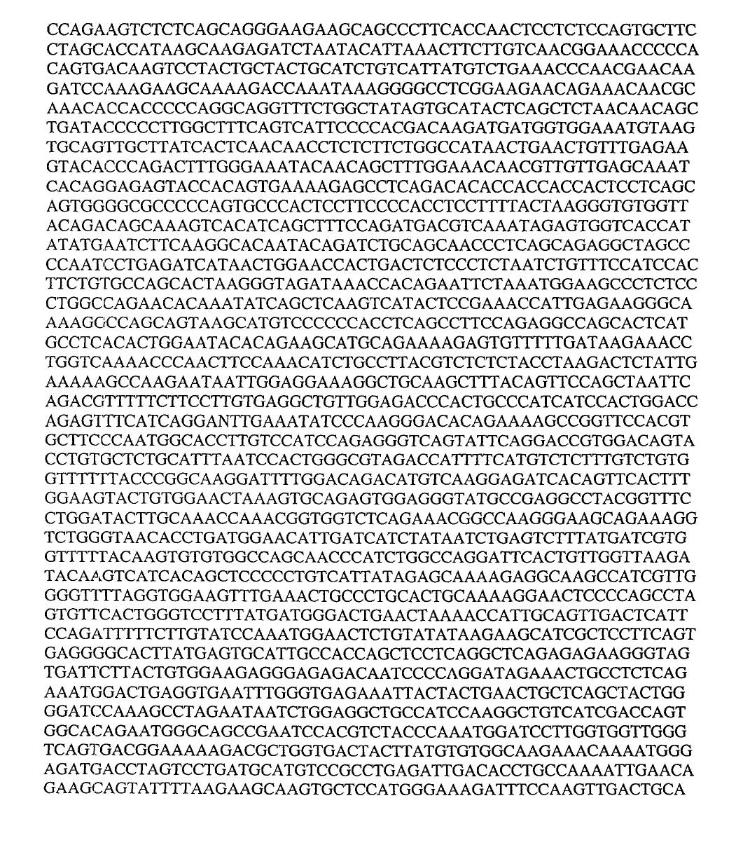

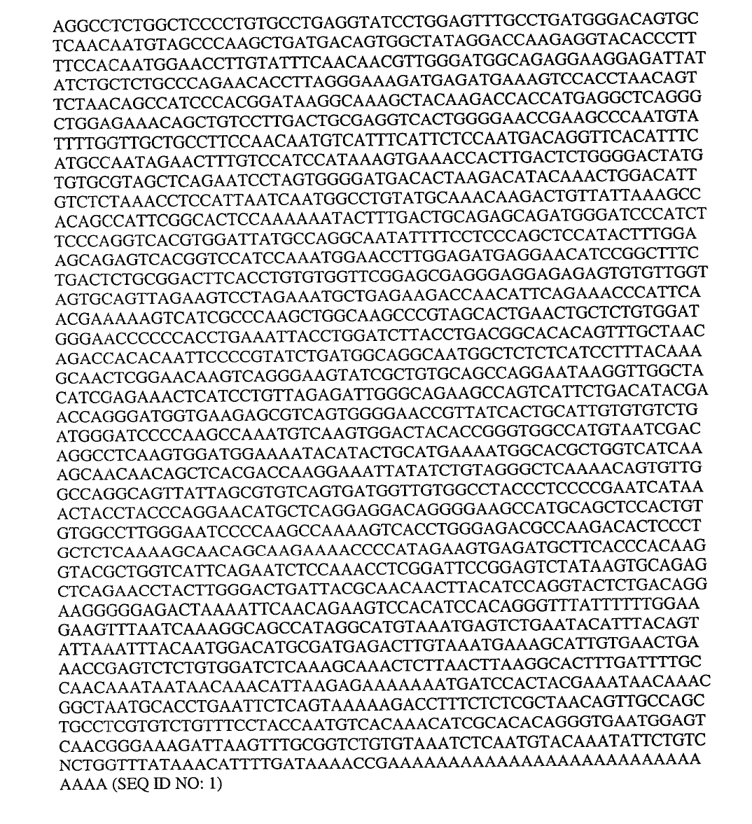

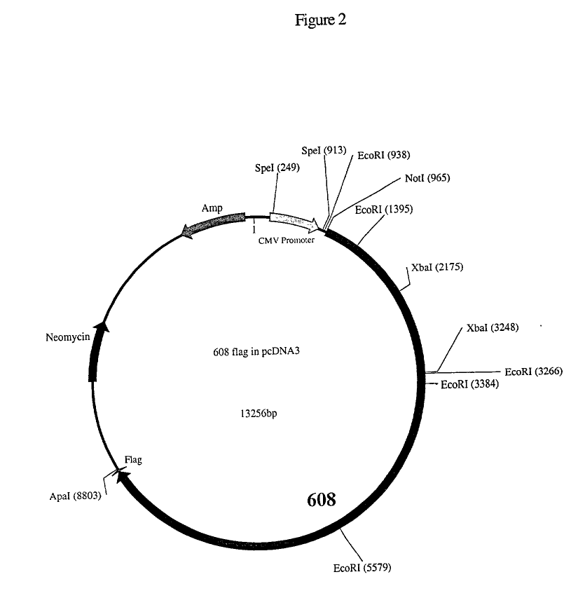

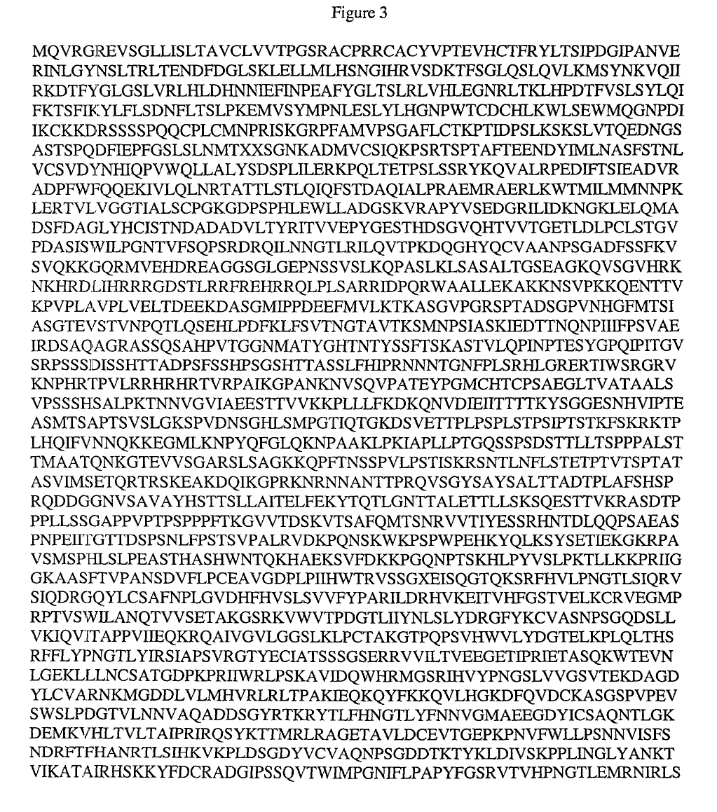

The partial OCP rat cDNA clone (4007 bp long) isolated from a rat calvaria cI)NA phage library was found to contain a 3356 bp open reading frame closed at the 3′ end. Comparison to public mouse databases revealed no sequence homologues. A complete OCP rat cDNA clone was isolated from the rat calvaria cDNA library by a combination of 5′ RACE technique (Clontech), RT-PCR of 5′ cDNA fragments, and ligation of the latter products to the original 3′ clone. The full rat cDNA clone that was generated (shown in FIG. 1 and pCDNA3.1-608, in FIG. 2) was sequenced, and no mutations were found. The full sequence stretch is 8883 bp long and contains an ORF (rit 575-8366) for a 2597 amino acid residue protein. FIG. 3. The cDNA does not contain a polyadenylation site, but contains a 3′ poly A stretch.

CMF608 encodes a large protein that appears to be a part of the extra-cellular matrix. The gene may be actively involved in supporting osteoblast differentiation. Another option is that it is expressed in regions were remodeling takes place. Such an hypothesis is also compatible with a role in directing osteoclast action and thus it may be a target for inhibition by small molecules.

In normal bone formation, activation of osteoblasts leads to secretion of various factors that attract osteoclast precursors or mature osteoclasts to the sites of bone formation to initiate the process of bone resorption. In normal bone formation both functions are balanced. Imbalance to any side causes either osteitis deformans (osteoblast function overwhelms) or osteoporosis (osteoclast function overwhelms).

Among known osteoblast activators—mechanical force stimulation—is actually applied in the present model. As proof of principle, increased expression of several genes known to respond to mechanical stress by transcriptional upregulation were found. They include tenascin, endothelin and possibly trombospondin.

### EXAMPLE 3

### Full-length OCP cDNA Construction and Expression

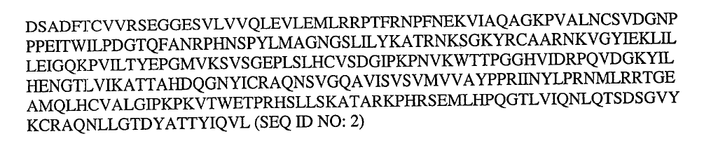

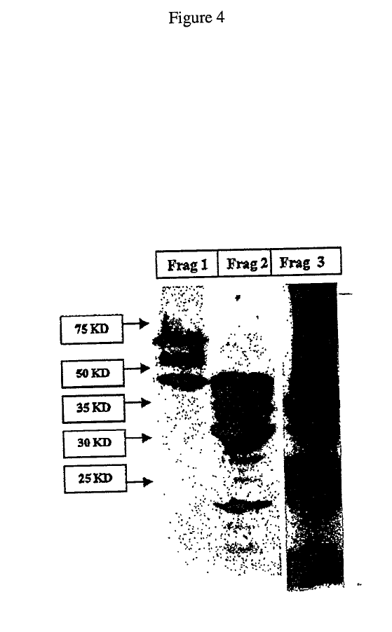

TNT (transcription—translation) assays were performed according to the manufacturer's instructions (Promega—TNT coupled reticulocyte lysate systems), using specific fragments taken from various regions of the gene. In all assays a clear translation product was observed. FIG. 4. The following fragments were tested:

TNT products

| Frag. | Location | Fragment size (bp) | Translation product size (kD) | Promoter |
|---|---|---|---|---|
| 1 | 134-2147 | 2013 | 73 | T7 |
| 2 | 3912-5014 | 1102 | 40 | ″ |
| 3 | 574-1513 | 939 | 34 | ″ |

### EXAMPLE 4

### The Mouse OCP Gene

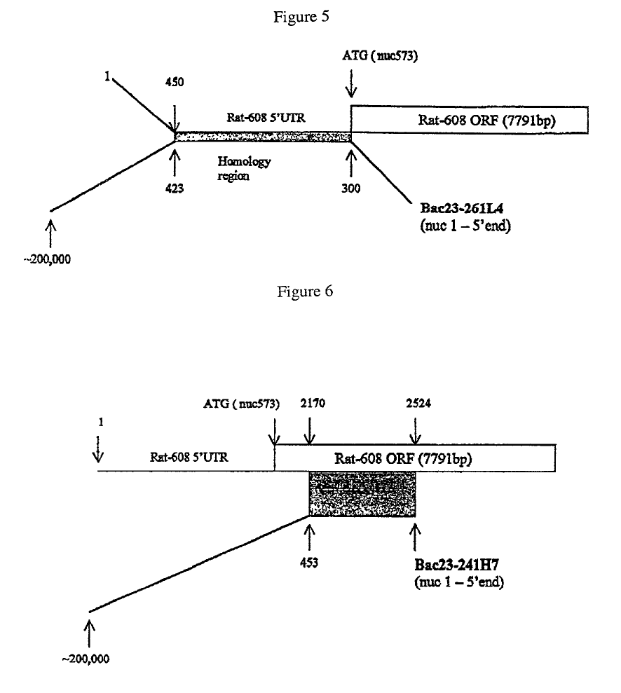

Two mouse genomic Bac clones containing the mouse OCP gene promoter region and part of the coding region were identified, based on their partial homology to the 5′UTR region of the rat-608 CDNA. These clones (23-261L4 and 23-241H7 with ˜200Kb average insert length) were bought from TIGR. FIGS. 5 and 6.

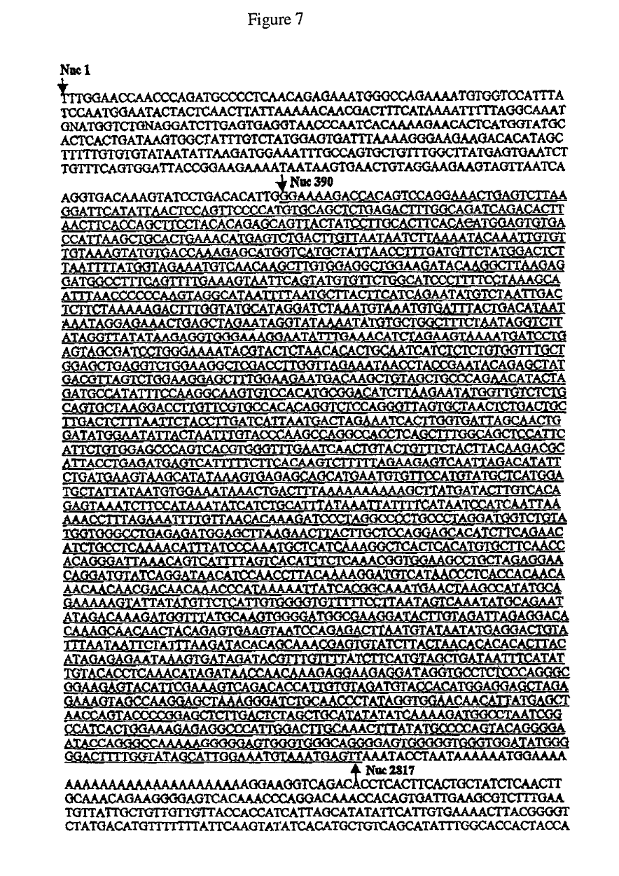

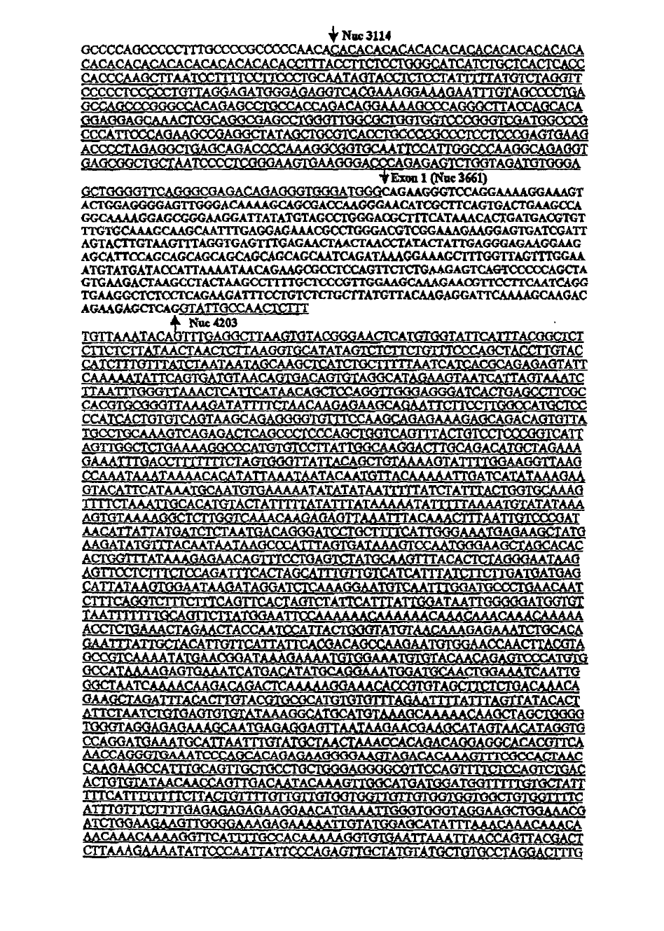

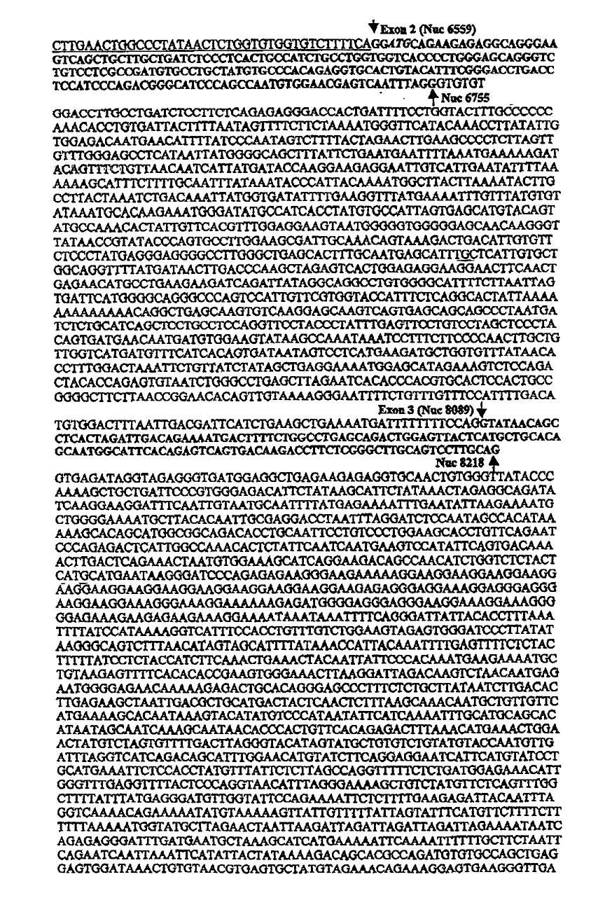

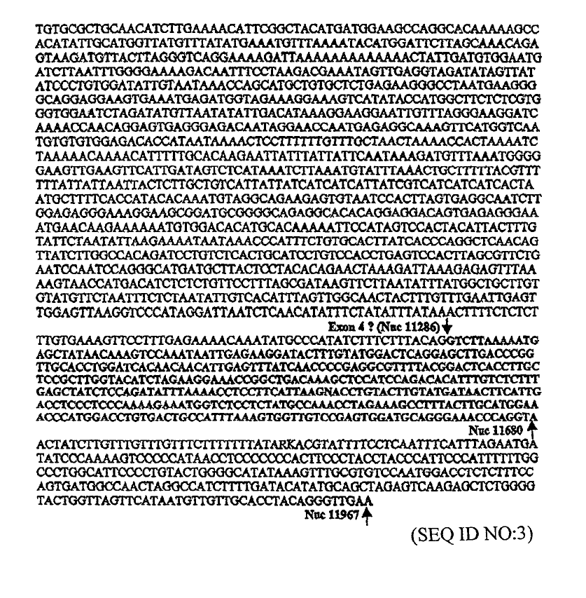

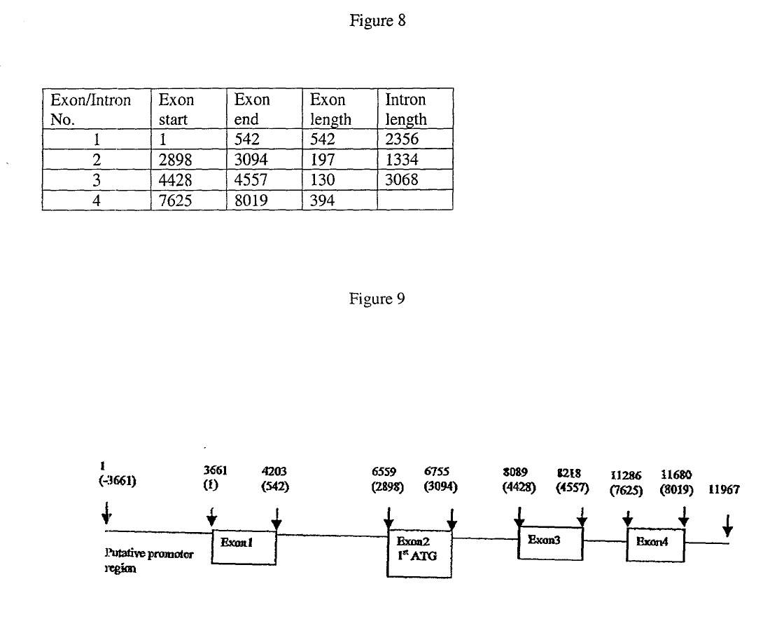

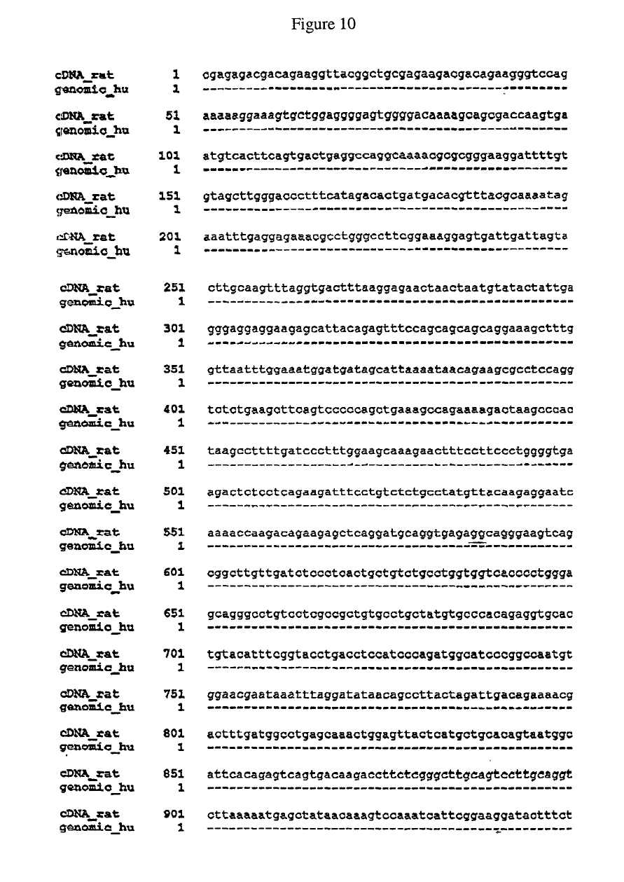

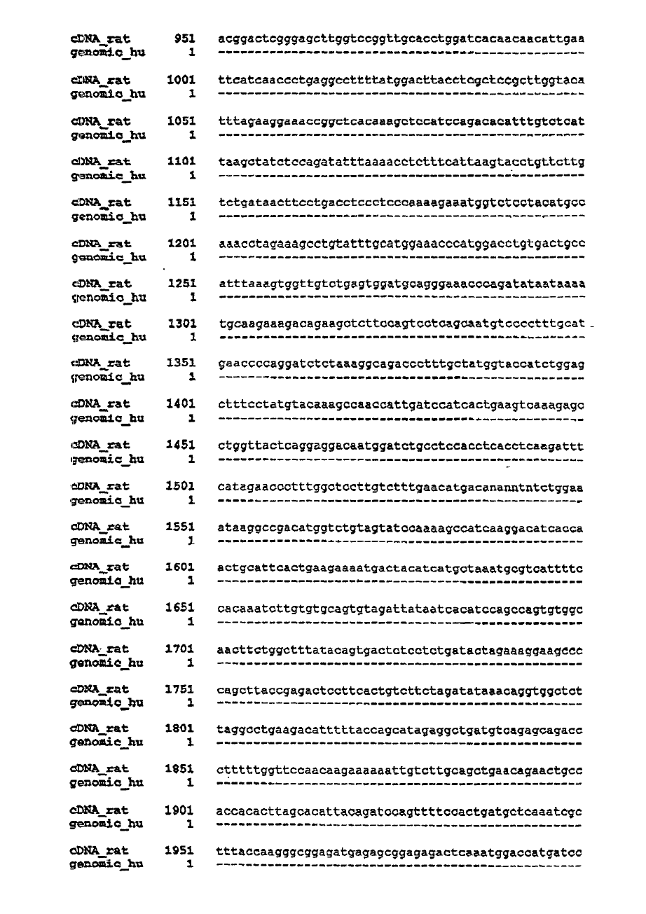

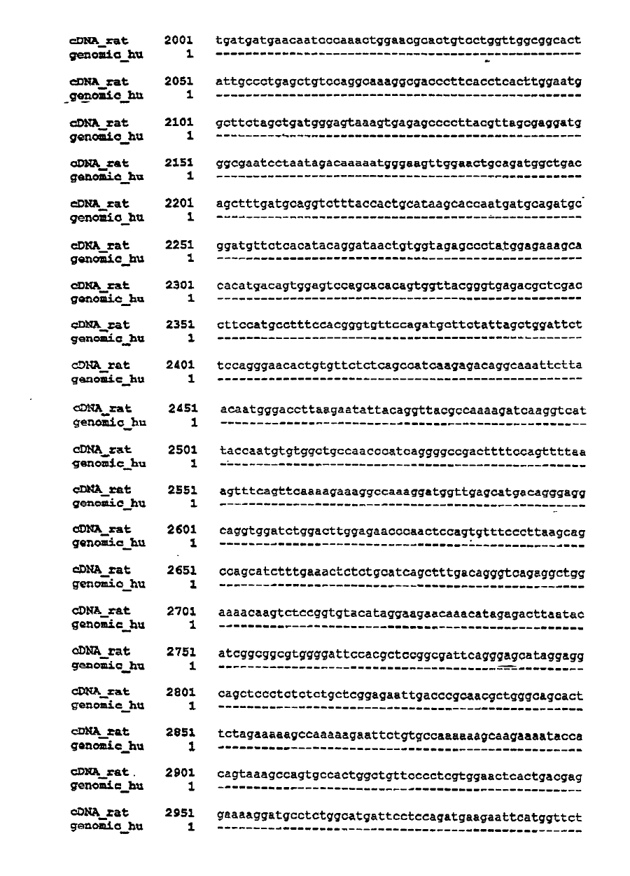

Specific primers for the amplification of a part of the mouse-OCP promoter region were designed and used for PCR screening of a mouse genomic phage library (performed by Lexicon Genetics Inc. for the Applicants). One phage clone containing part of the genomic region of the mouse 608 gene was detected and completely sequenced. The length of this clone was reported to be 11,963 bp. Parts of the physical “Lexicon” clone were re-sequenced by the inventors and corrections were made. The resequenced clone (FIG. 7) is 11967 bp long. Exon-location prediction (FIG. 8) was performed by the Applicant company's Bioinformatics unit based on the alignment of the mouse genomic and the rat cDNA sequences. FIGS. 9 and 10, respectively.

### EXAMPLE 5

### The Human OCP Gene

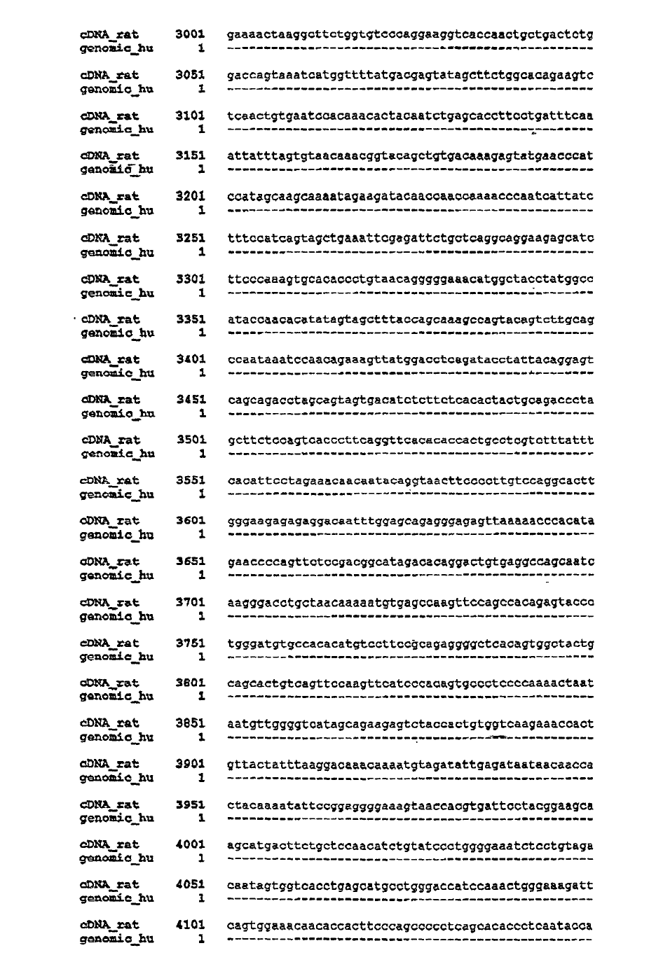

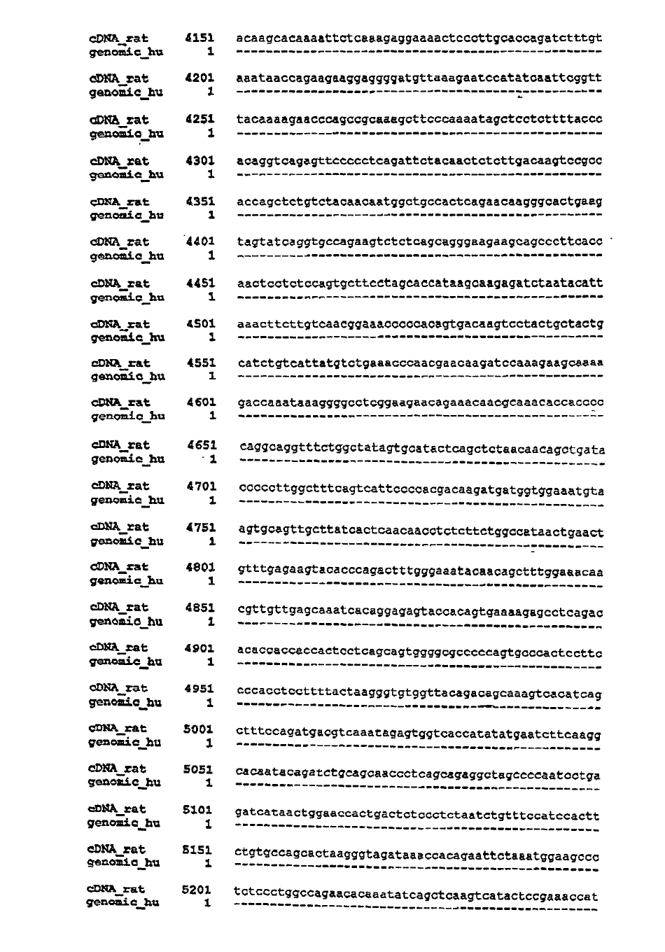

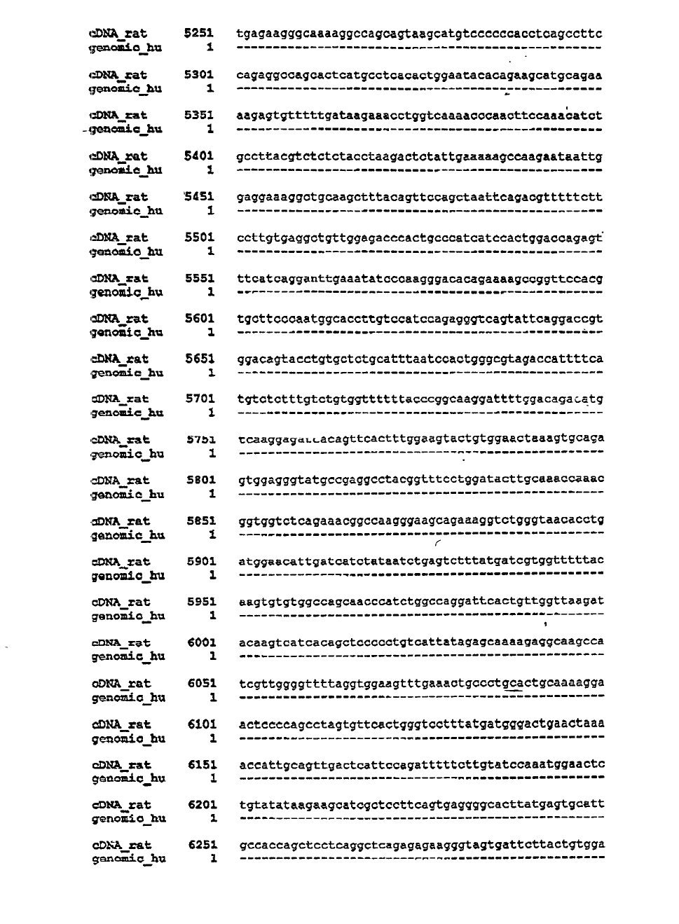

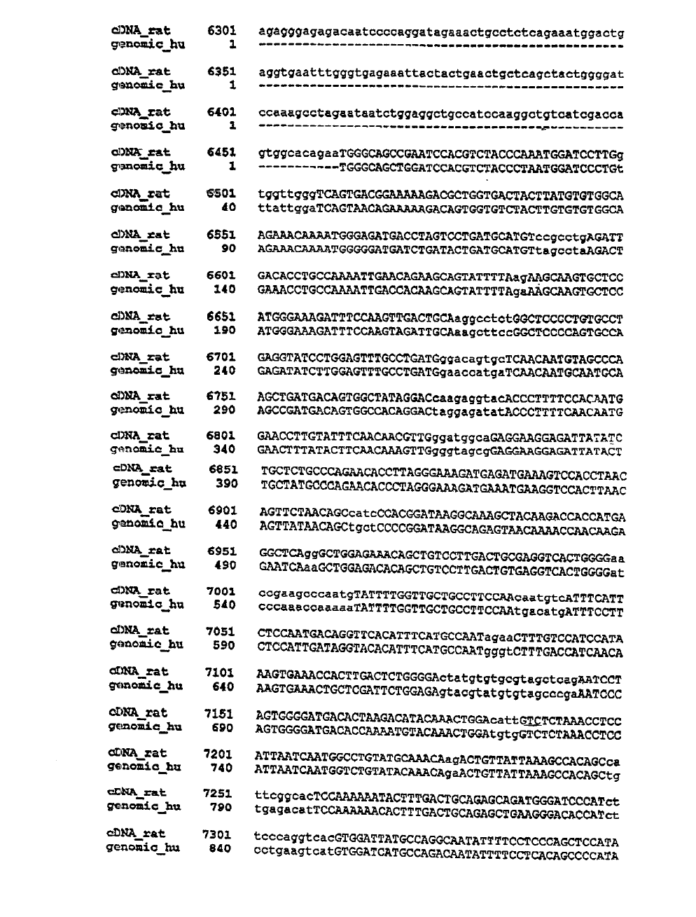

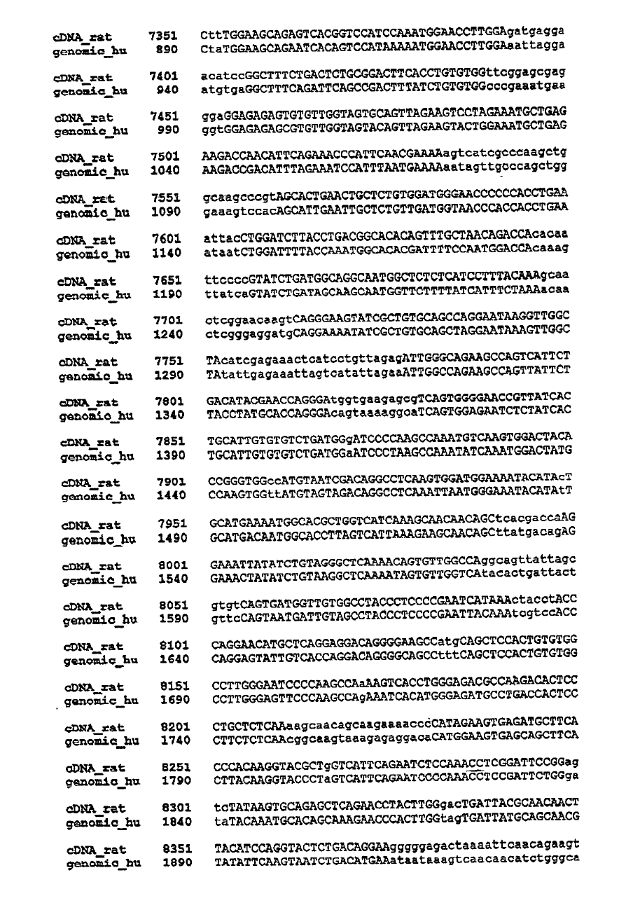

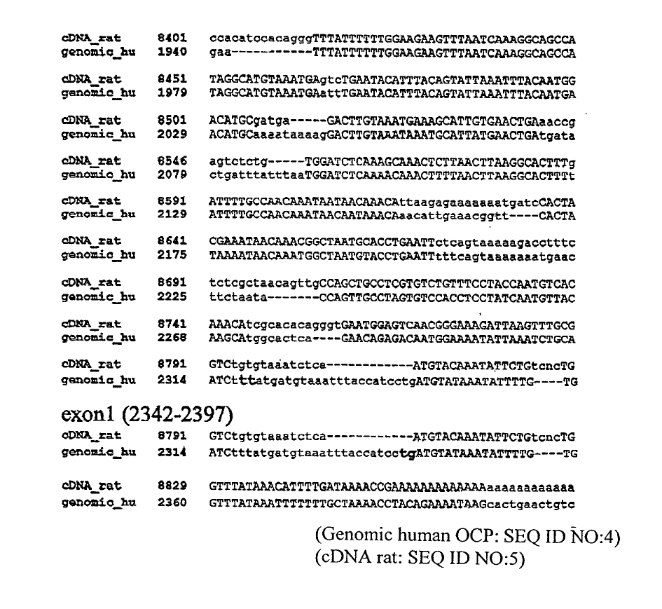

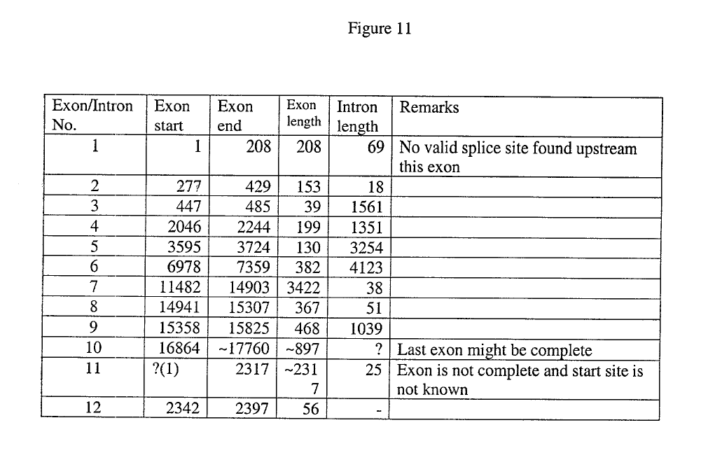

On the nucleotide level, the rat OCP cDNA sequence is homologous to the human genomic DNA sequence located on chromosome 3. Based on the homology and bioinformatic analysis (FIGS. 10 and 11), a putative cDNA sequence was generated.

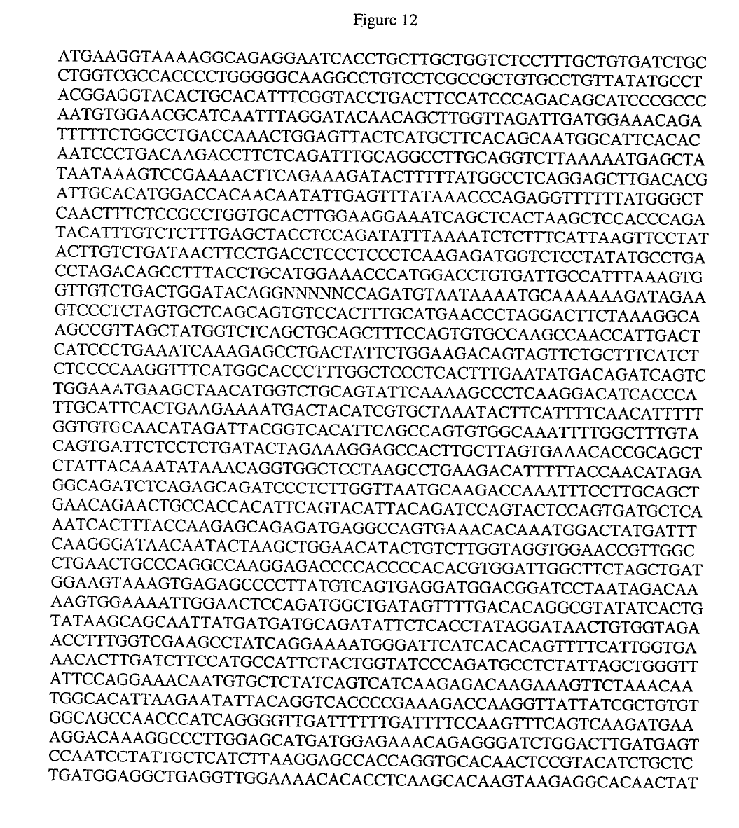

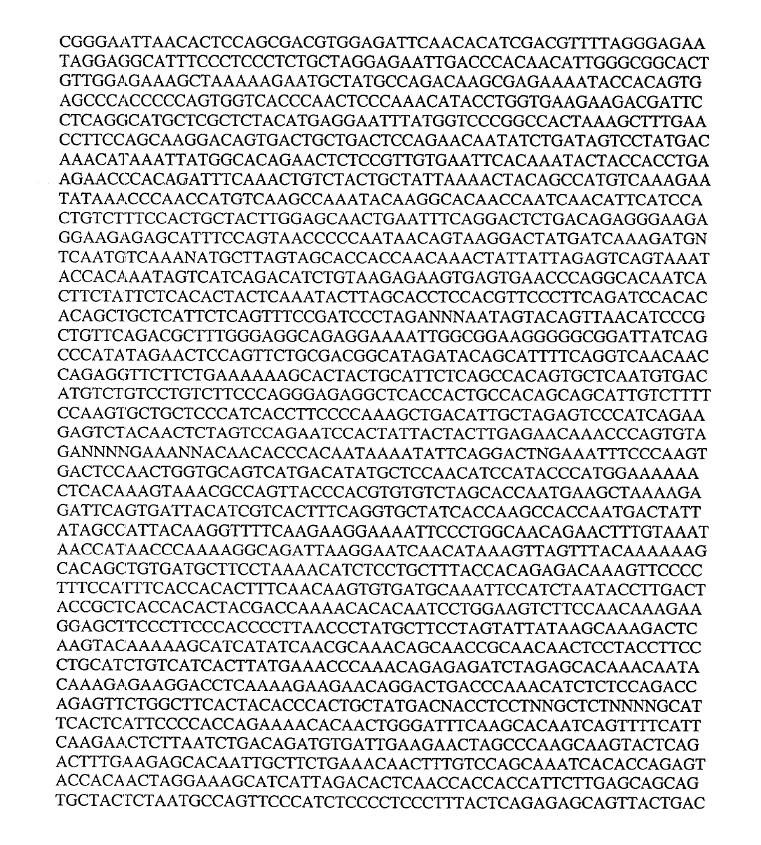

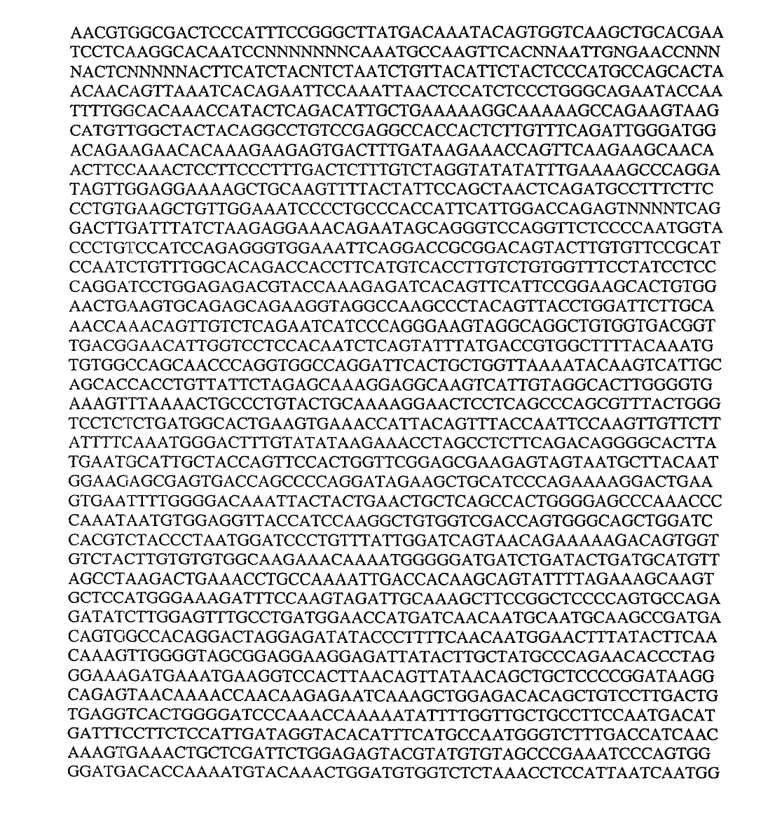

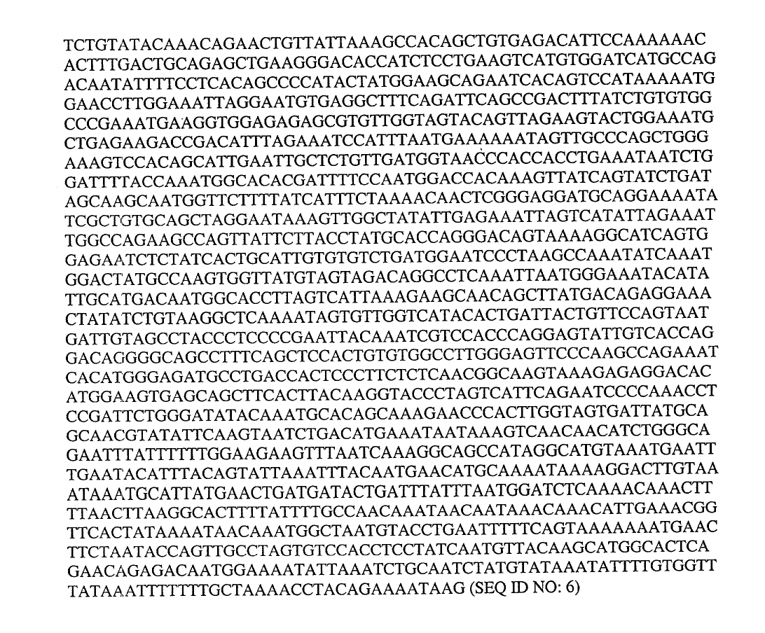

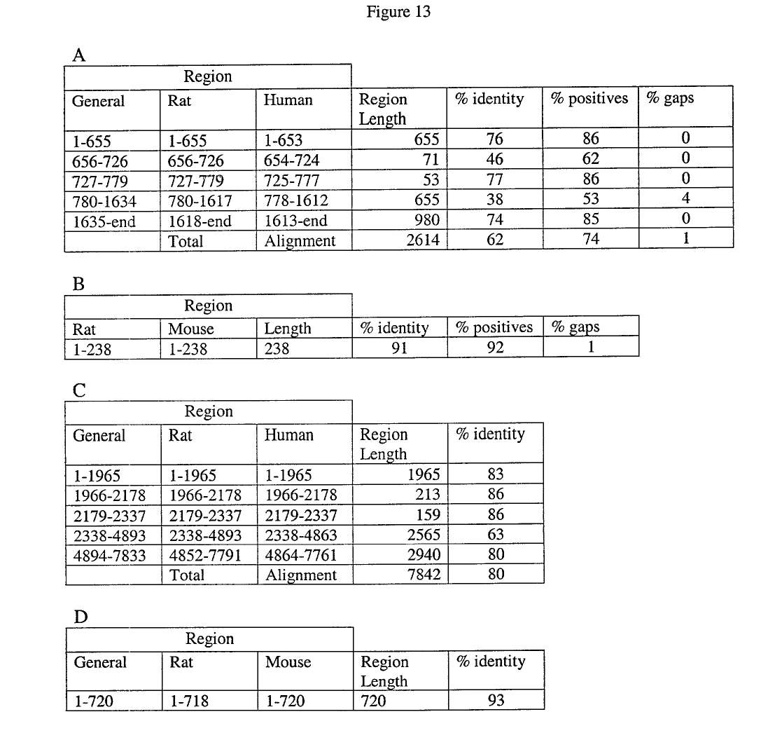

FIG. 12. The highest similarity is evident between nt 1-1965 (1-655 a.a); 2179-2337 (727-779 a.a); and 4894-7833 (1635 a.a.-end) as presented in the table shown in FIG. 13. On the protein level, no homologues were found in the data bank.

### EXAMPLE 6

### The Deduced OCP Protein

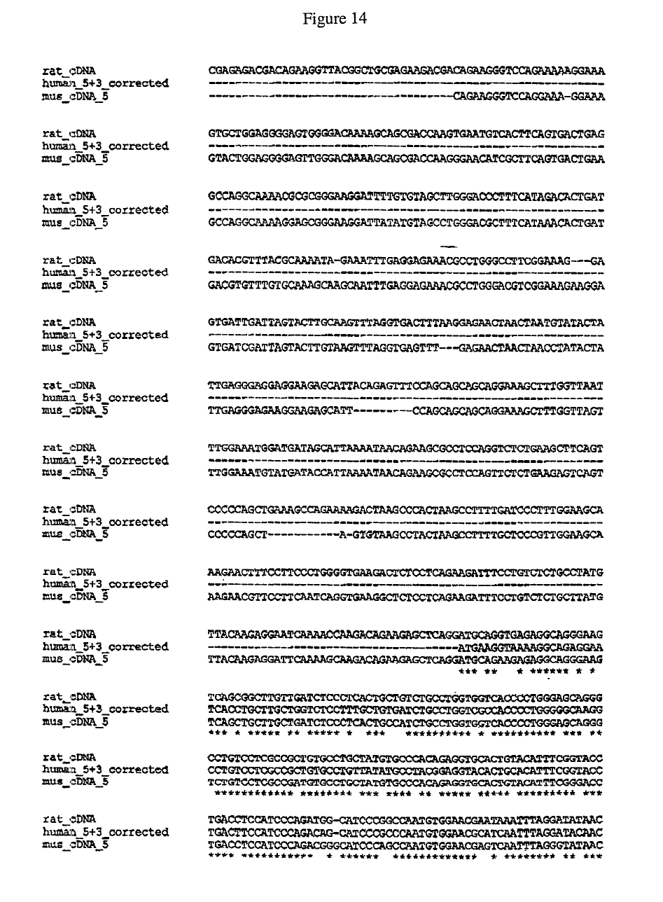

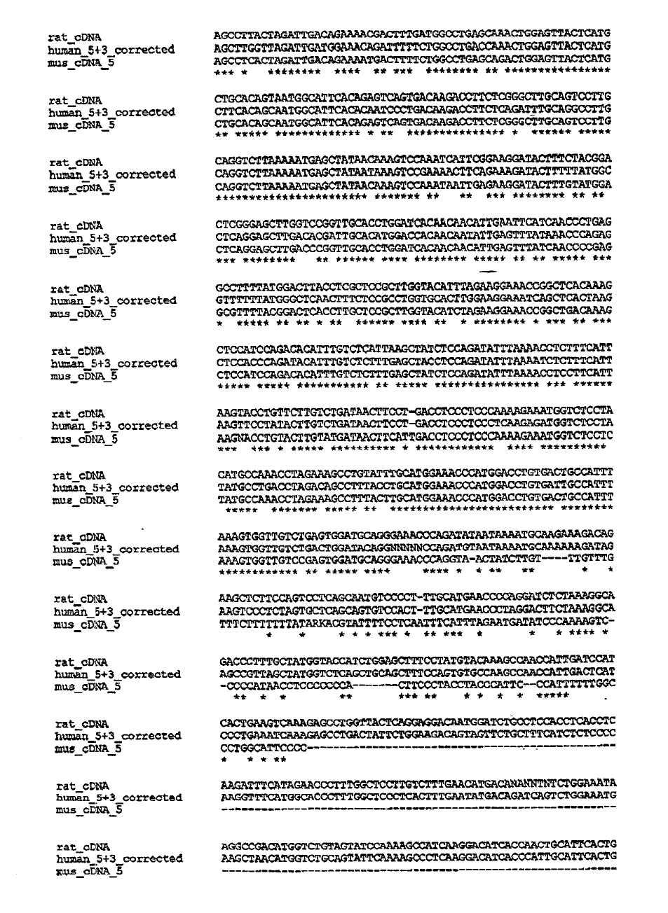

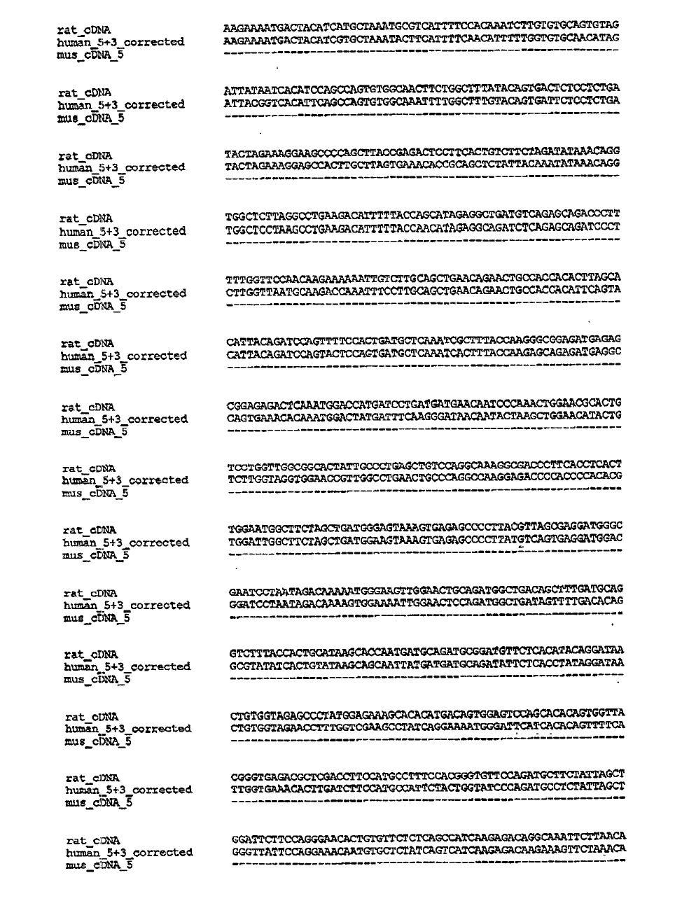

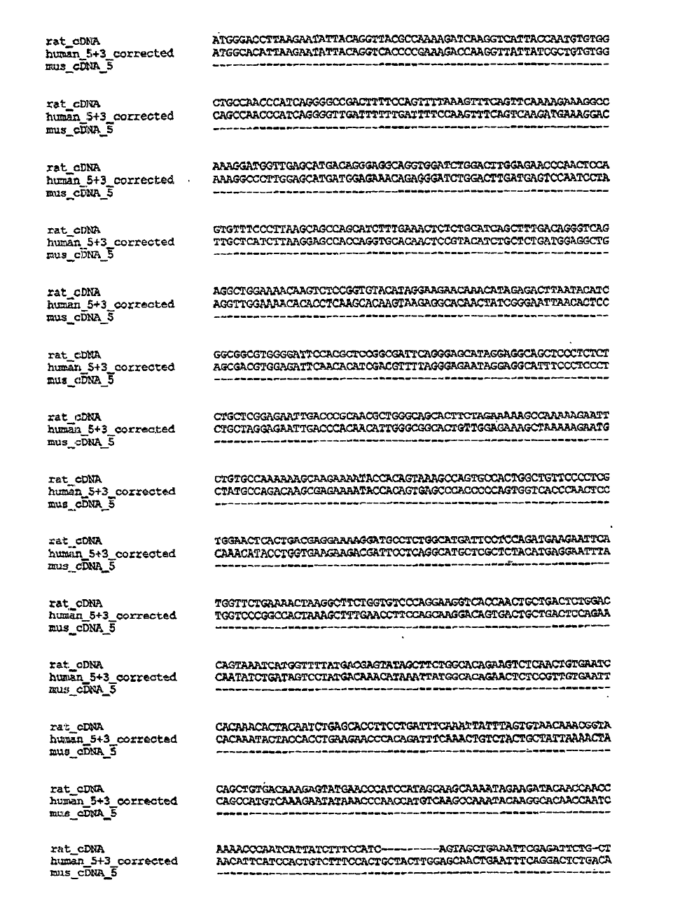

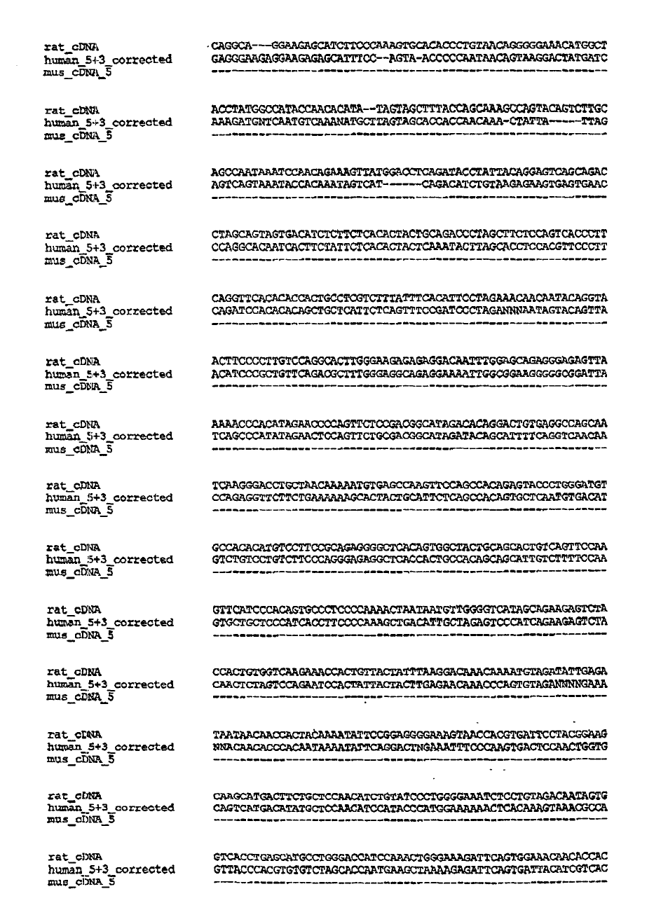

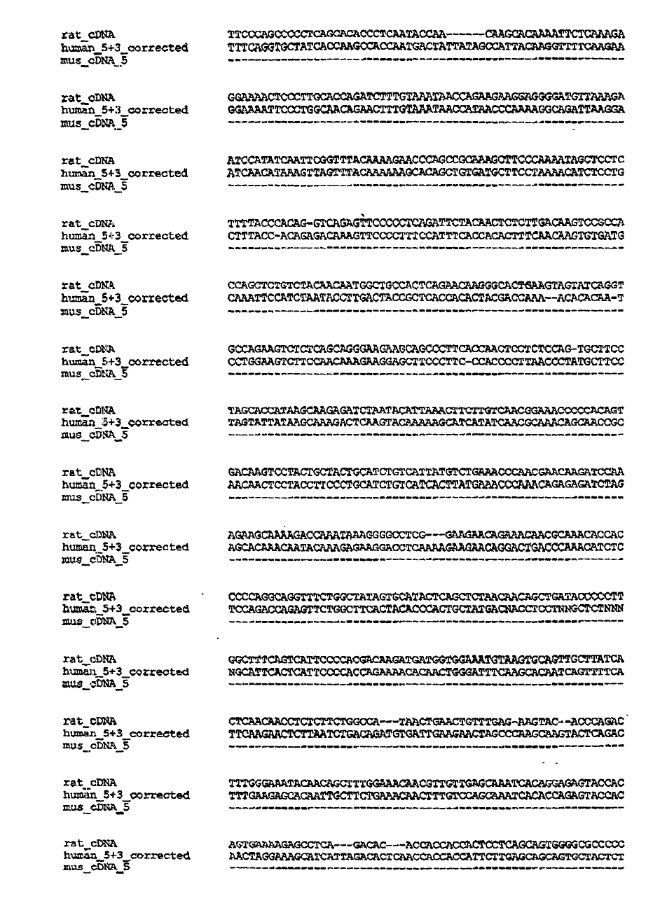

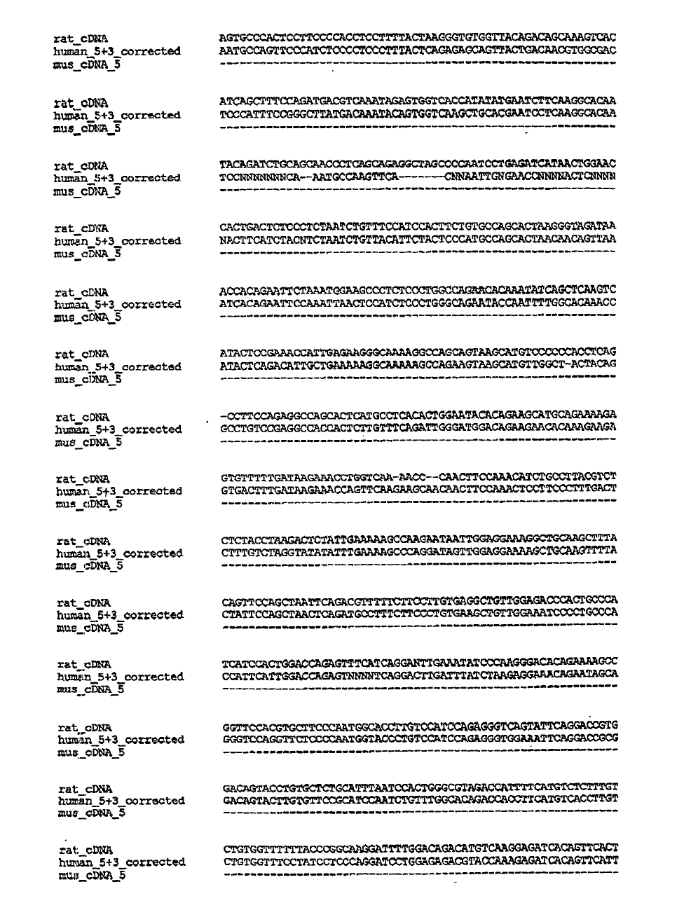

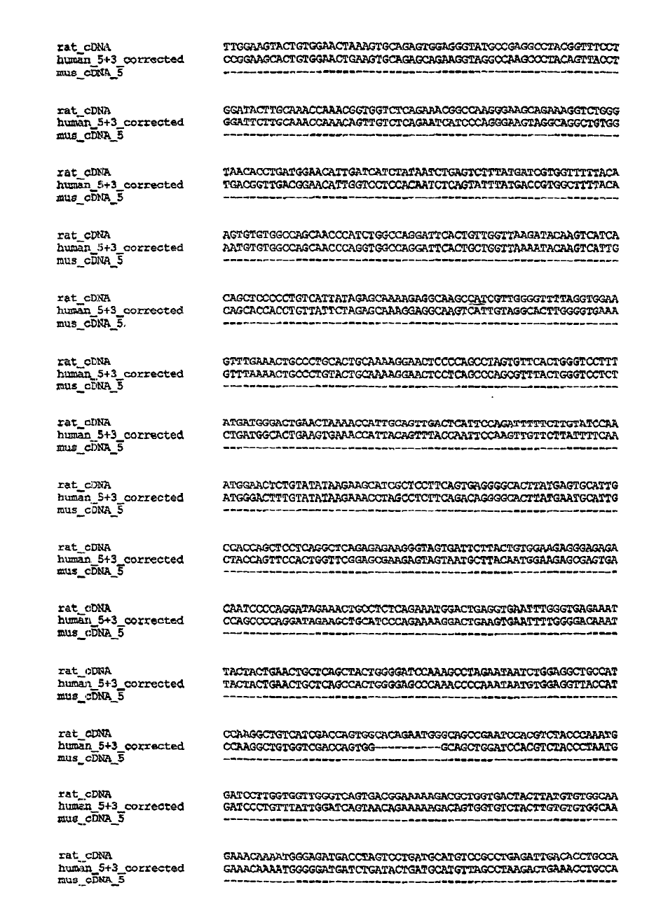

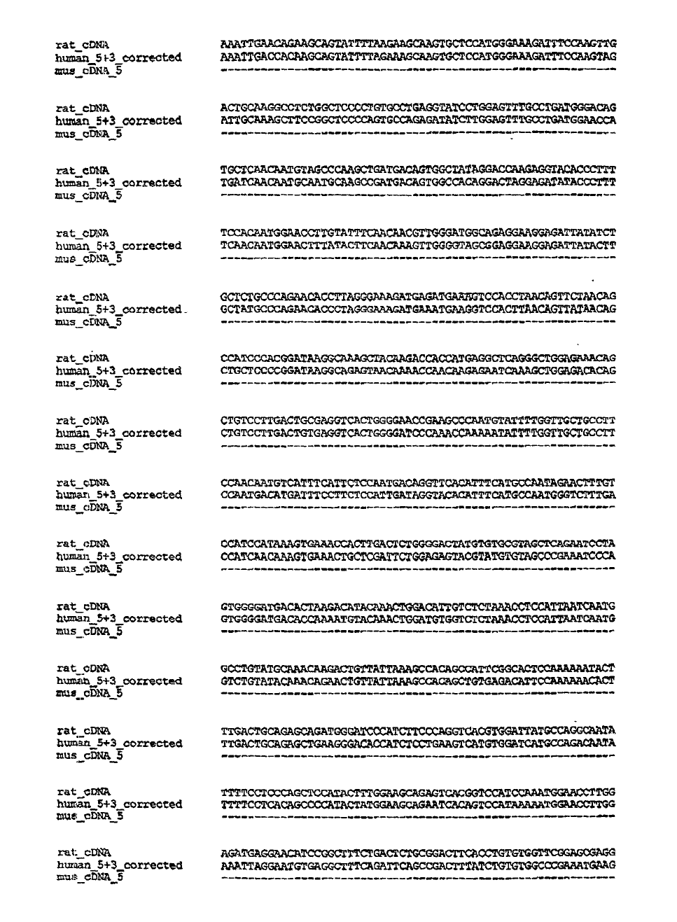

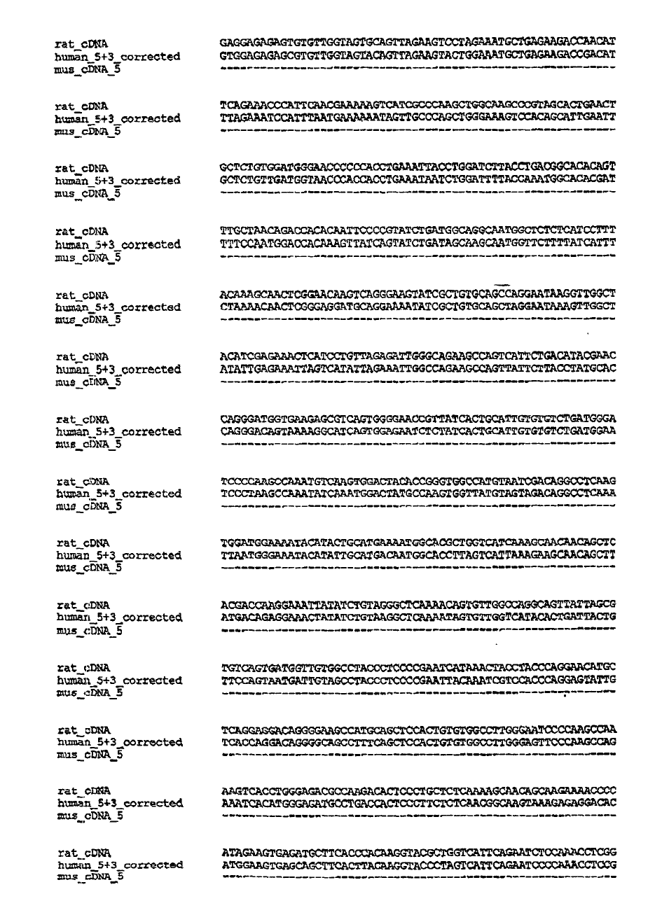

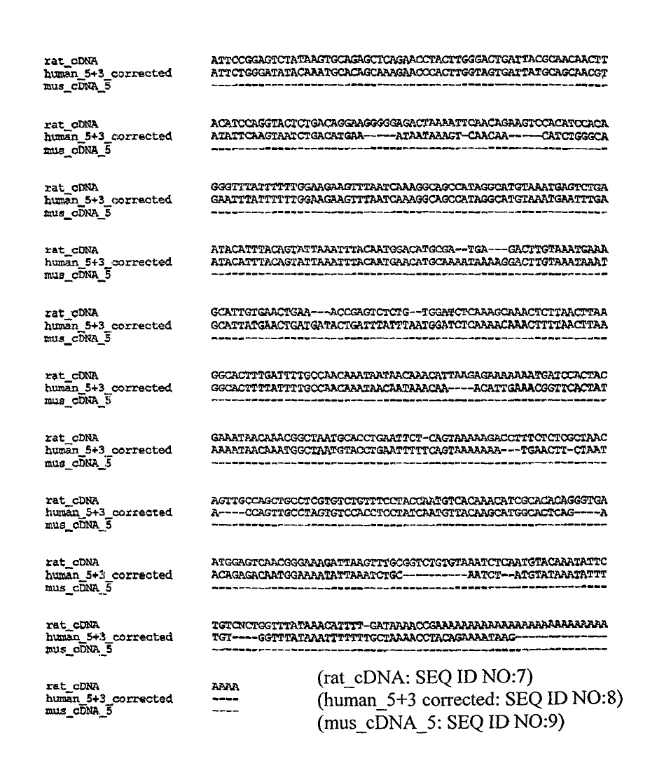

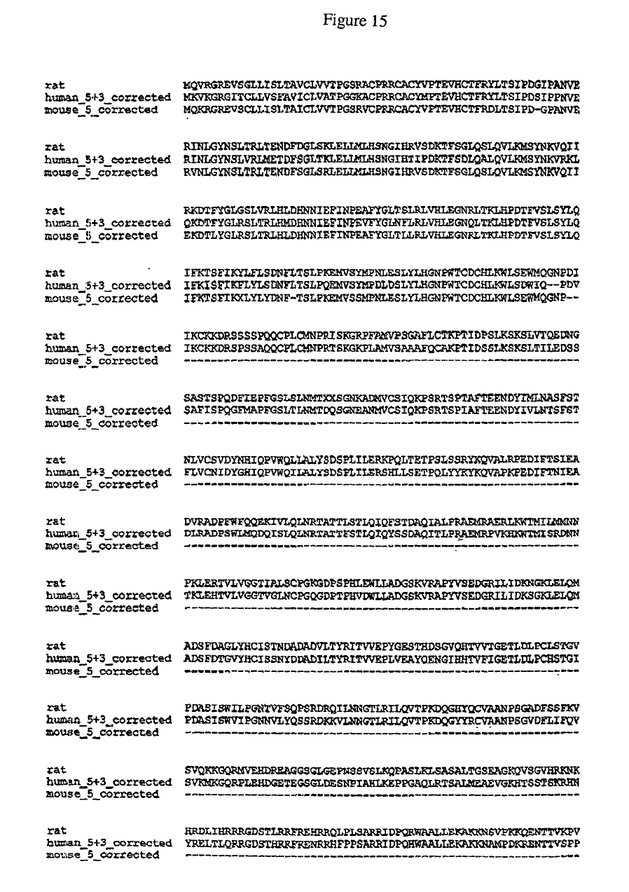

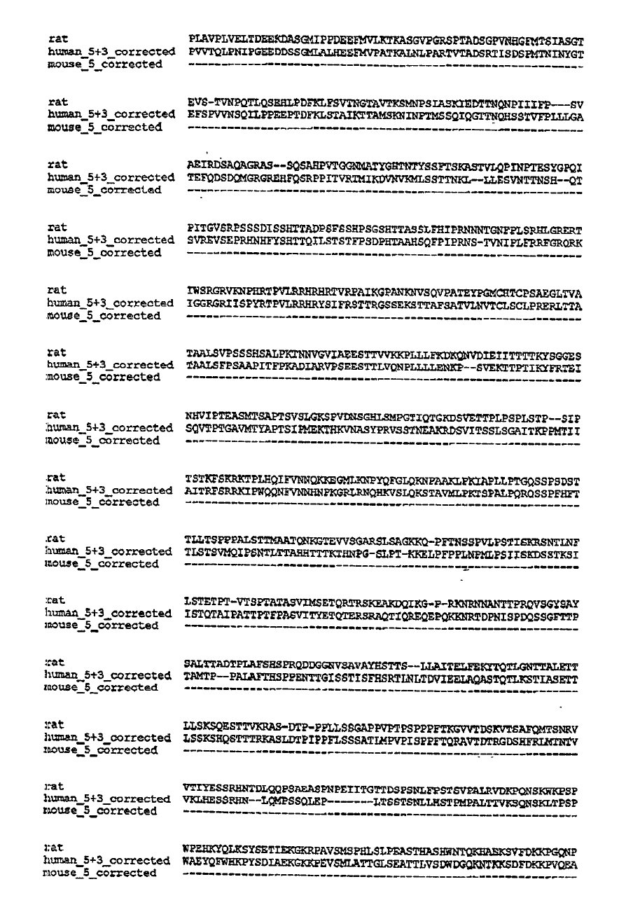

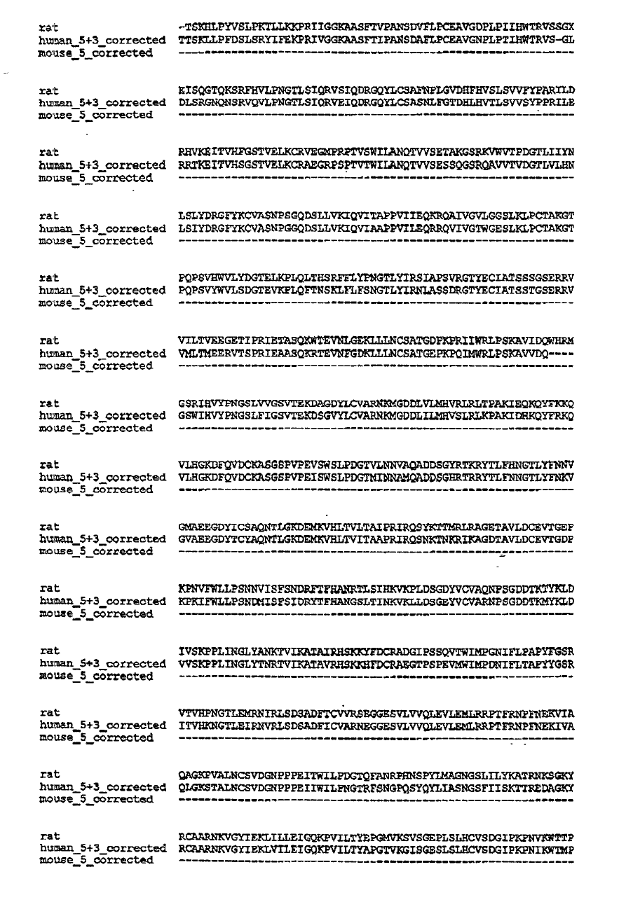

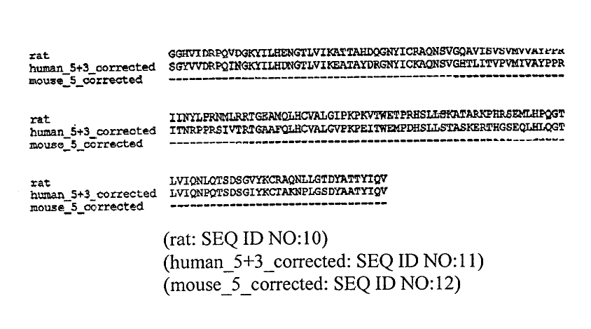

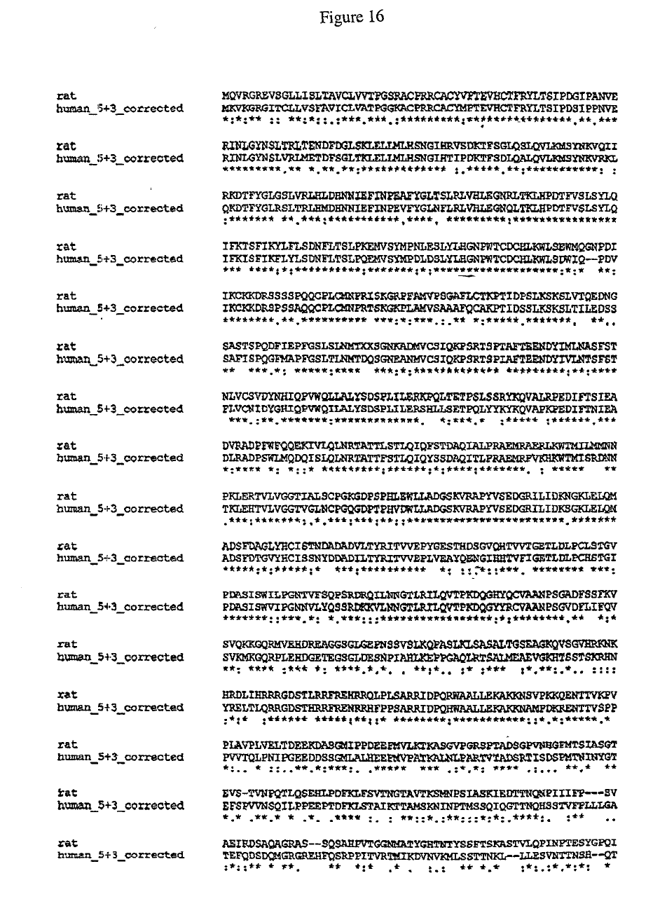

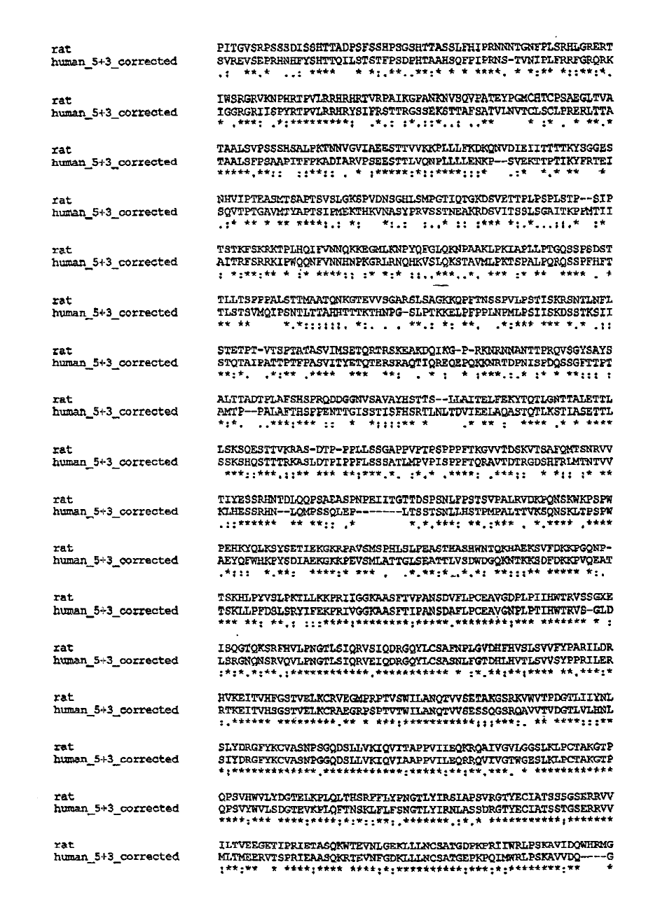

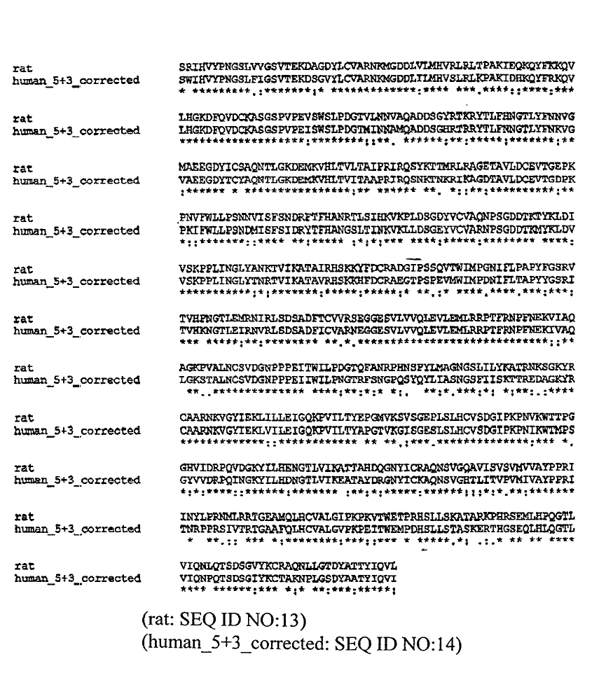

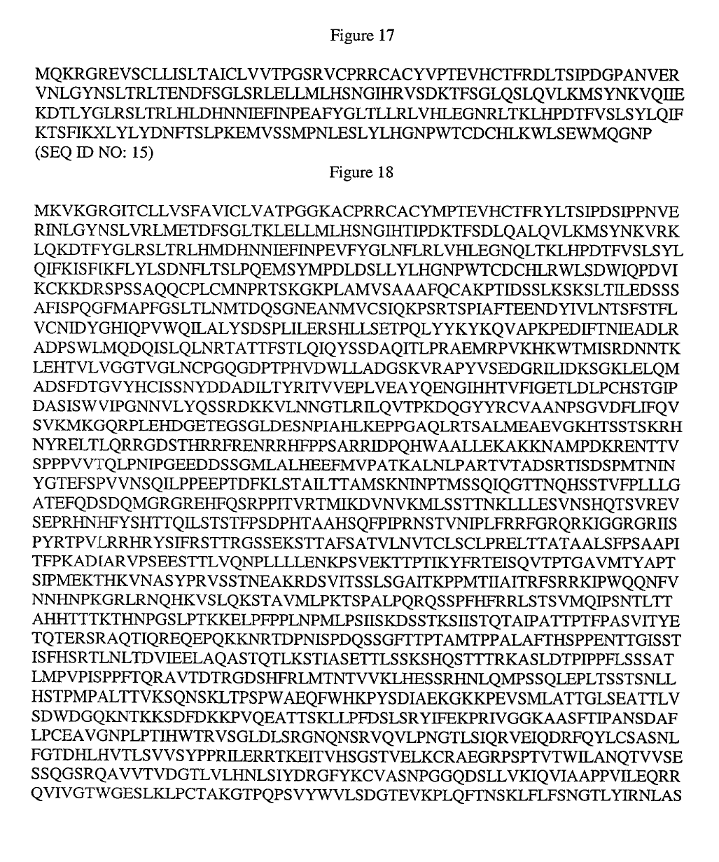

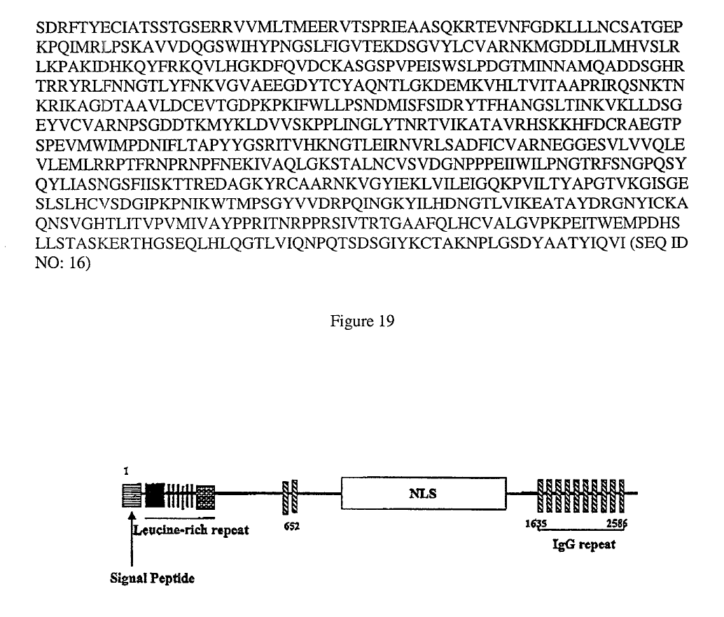

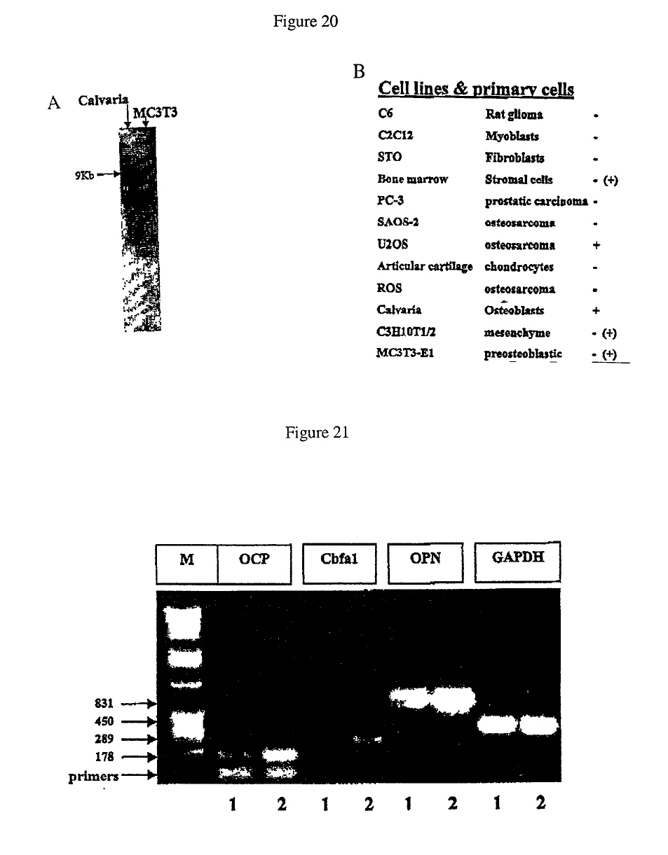

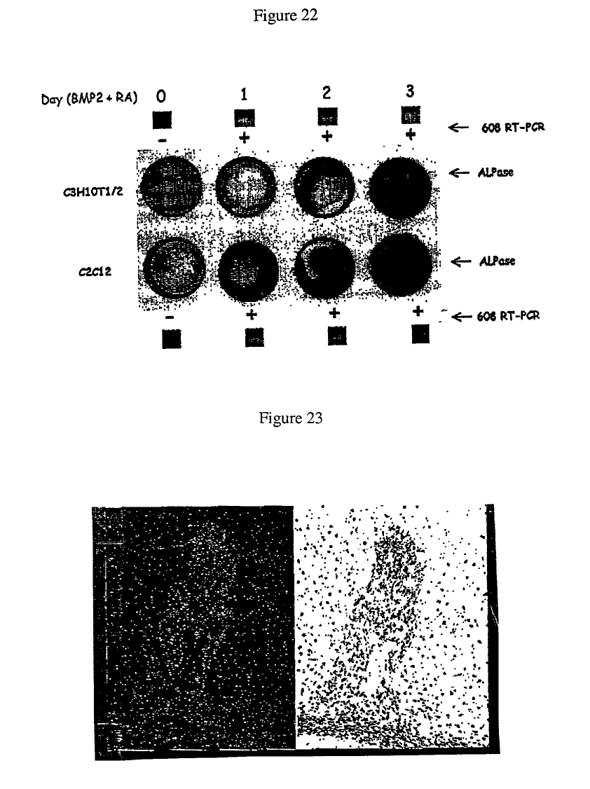

The deduced OCP protein was generated following the alignment (FIGS. 14-16) of the rat, mouse and human cDNA sequences (FIGS. 1, 7 and 12, respectively) and the equivalent rat, mouse and human amino acid sequences (FIGS. 3, 21 and 22, respectively).

The deduced OCP protein contains the following features (FIG. 18):

a. a cleavable, well-defined N-terminal signal peptide (aa 1-28);

b. a leucine-rich repeat region (aa 28-280). This region can be divided into N-terminal and C-terminal domains of leucine-rich repeats (aa 28-61 and 219-280, respectively). Between them, there are six leucine-rich repeat outliers (aa 74-96, 98-120, 122-144, 146-168, 178-200, 202-224). Leucine rich repeats are usually found in extracellular portions of a number of proteins with diverse functions. These repeats are thought to be involved in protein-protein interactions. Each leucine-rich repeat is composed of β-sheet and α-helix. Such units form elongated non-globular structures;

c. twelve immunoglobulin C-2 type repeats at amino acid positions 488-558, 568-652, 1635-1704, 1732-1801, 1829-1898, 1928-1997, 2025-2100, 2128-2194, 2233-2294, 2324-2392, 2419-2487, 2515-2586. Thus, two Ig-like repeats are found immediately downstream of a leucine-rich region, while the remaining 10 repeats are clustered at the protein's C-terminus. Immunoglobulin C-2 type repeats are involved in protein—protein interaction and are usually found in extracellular protein portions;

d. no transmembrane domain; and

e. 5 nuclear localization domains (NLS) at: 724, 747, 1026, 1346 and 1618.

These observations indicate that OCP belongs to the Ig superfamily. OCP is a serine-rich protein (10.3% versus av. 6.3%), with a central nuclear prediction domain and an N-terminal extracellular prediction domain.

### EXAMPLE 7

### Bone Fracture Healing

Expression of 608 RNA is bone-specific. Moreover, it seems to be specific to bone progenitors (as judged by their location in bone and involvement in normal bone modeling and remodeling processes) that do not yet express the known bone-specific markers. To further prove the relevance of 608-expressing cells to osteogenic lineage, the patterns of 608 expression in the animal model of bone fracture healing that imply the activation of bone formation processes were studied.

The sequence of physiological events following bone fracture is now relatively well understood. Healing takes place in three phases—inflammatory, reparative and remodeling. In each phase certain cells predominate and specific histological and biochemical events are observed. Although these phases are referred to separately, it is well known that events described in one phase persist into the next and events apparent in a subsequent phase begin before this particular phase predominates. These events have been described over the years in investigative reports and review articles. Ham (1969) In, Histology, 6th ed. Philadelphia, Lippincott, p. 441; and Urist and and Johnson (1943) J. Bone Joint Surg. 25:375.

During the first phase immediately following fracture (the inflammatory phase), wide-spread vasodilatation and exudation of plasma lead to the acute edema visible in the region of a fresh fracture. Acute inflammatory cells migrate to the region, as do polymorphonuclear leukocytes and then macrophages. The cells that participate directly in fracture repair during the second phase (the reparative phase), are of mesenchymal origin and are pluripotent. These cells form collagen, cartilage and bone. Some cells are derived from the cambium layer of the periosteum and form the earliest bone. Endosteal cells also participate. However, the majority of cells directly taking part in fracture healing enter the fracture site with the granulation tissue that invades the region from surrounding vessels. Trueta (1963) J. Bone Joint Surg. 45:402. Note that the entire vascular bed of an extremity enlarges shortly after the fracture has occurred but the osteogenic response is limited largely to the zones surrounding the fracture itself. Wray (1963) Angiol. 14:134.

The invading cells produce tissue known as “callus” (made up of fibrous tissue, cartilage, and young, immature fibrous bone), rapidly enveloping the ends of the bone, with a resulting gradual increase in stability of the fracture fragments. Cartilage thus formed will eventually be resorbed by a process that is indistinguishable except for its lack of organization from endochondral bone formation. Bone will be formed by those cells having an adequate oxygen supply and subjected to the relevant mechanical stimuli.

Early in the repair process, cartilage formation predominates and glycosaminoglycans are found in high concentrations. Later, bone formation is more obvious. As this phase of repair takes place, the bone ends graually become enveloped in a mass of callus containing increasing amounts of bone. In the middle of the reparative phase the remodeling phase begins, with resorption of portions of the callus and the laying down of trabecular bone along lines of stress. Finally, exercise increases the rate of bone repair. Heikkinen et al. Scand J. Clin. Lab. Invest. 25(suppl 113):32. In situ hybridization results have shown that OCP expression is confined to very specific regions where bone and cartilage formation is initiated.

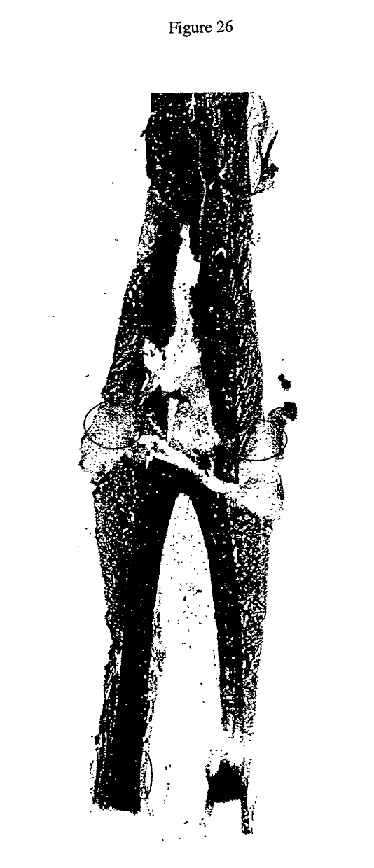

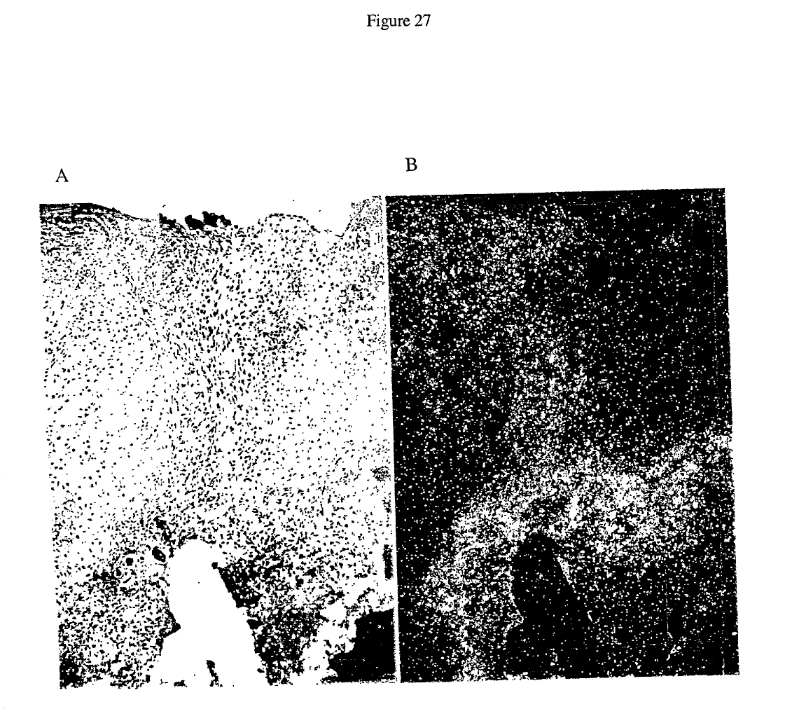

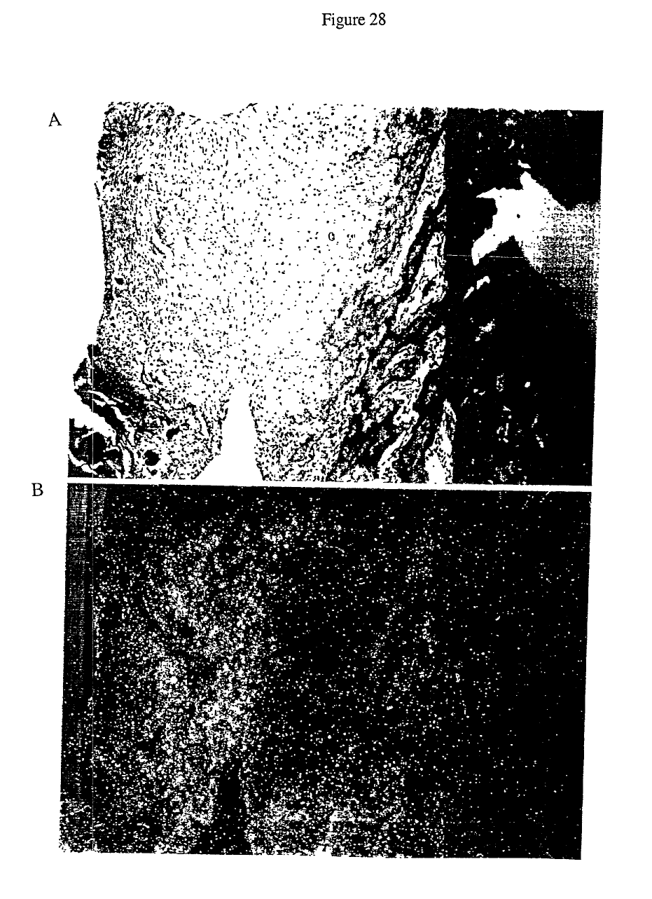

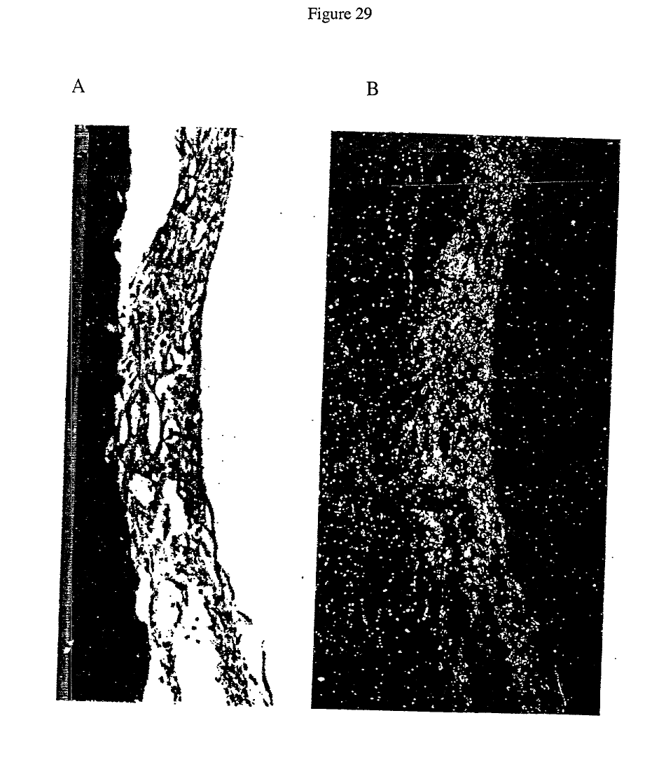

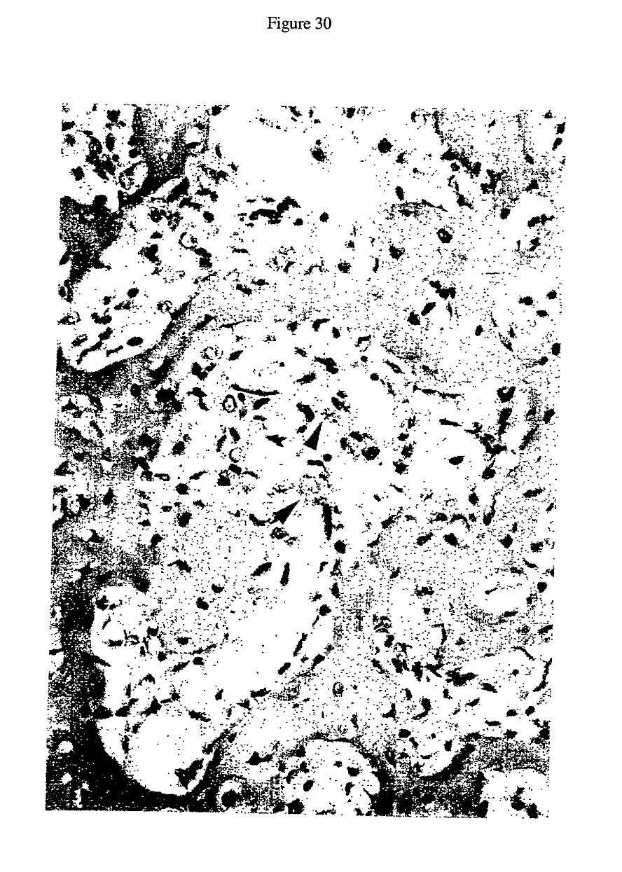

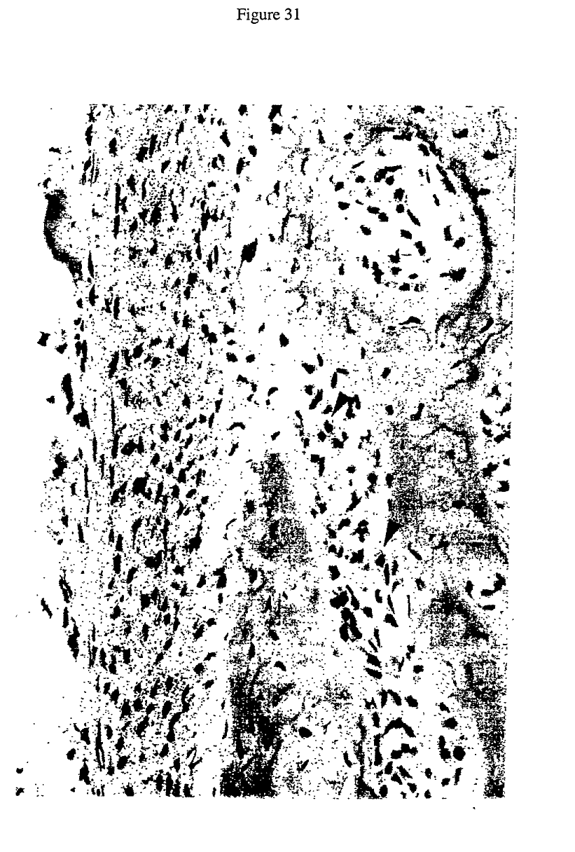

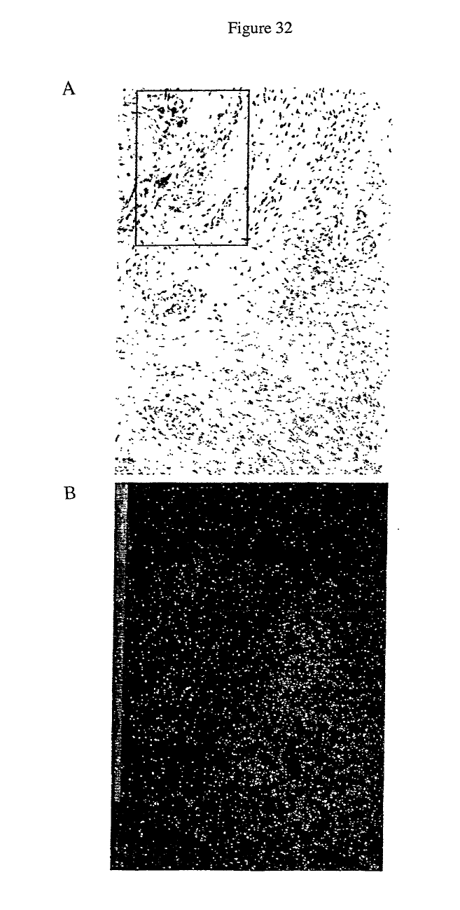

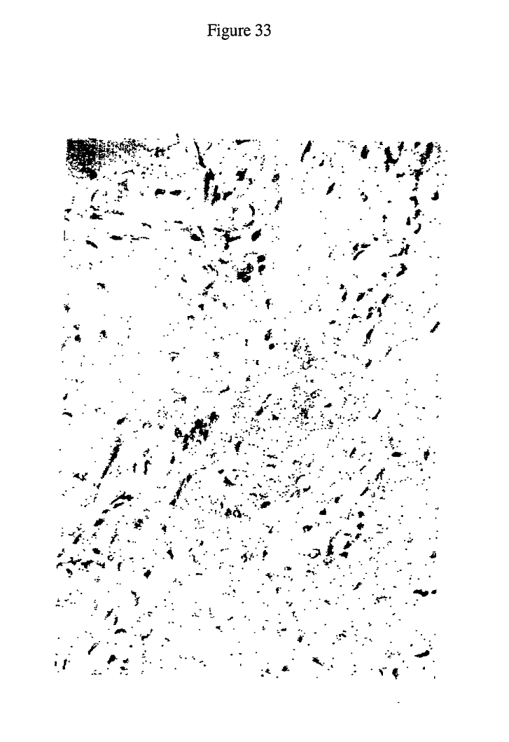

In order to find out if OCP expression is induced in an animal model of bone fracture healing, a standard midshaft fracture was created in rat femur by means of a blunt guillotine, driven by a dropped weight. Bonnarens et al. (1984) Orthop. Res. 2:97-101. One, 2, 3 and 4 week-fractured bones were excised, fixed in buffered formalin, decalcified in EDTA solution and embedded in paraffin. All sections were hybridized with the OCP probe. The in-situ hybridization results show that a strong hybridization signal was apparent during the first and second weeks of fracture healing in the highly vascularized areas of the connective tissue within the callus (FIGS. 26-28), the endosteum (FIG. 29), the woven bone (FIG. 30) and the periosteum (FIG. 31). The periosteum is regarded as a source of undifferentiated progenitors participating in callus formation at the site of bone fracture. The hybridization signal disappeared slowly during further differentiation stages of fracture healing (three and four weeks) and was retained only in the vascularized connective tissue. FIG. 32 displays brightfield (left) and darkfield (right) microphotographs of a section of fractured bone healed for 4 weeks. In these later healing stages, the mature callus tissue was found to be comprised mainly by cancellous bone undergoing remodeling into compact bone, with little if any cartilage or woven bone present. The volume of the vascularized periosteal tissue is decreased but multiple cells in the periosteal tissue area of active remodeling of the cancellous bone covering the callus, show hybridization signal. This tissue covers the center of the callus and is also entrapped within the bone. See FIGS. 32 and 33. The box in FIG. 32 is enlarged in FIG. 33. As in the earlier stages, no hybridization signal was found in chondrocytes and osteoblasts. FIGS. 27 and 33. Several OCP expressing cells are concentrated in the vascular tissue that fills the cavities resulting from osteoclast activity (marked by asterisks).

Fractures in the young heal rapidly, while adult bone fractures heal slowly. The cause is a slower recruitment of specific chondro-/osteo-progenitors for the reparative process. Denervation retards fracture healing by diminishing the stress across the fracture site, while mechanical stress increases the rate of repair probably by increasing the proliferation and differentiation of specific bone progenitor cells and as a result, accelerates the rate of bone formation. The above results confirm our conclusions (see also hereunder) that OCP is most probably involved in induction of cortical and trabecular bone formation and remodeling, endochondral bone growth during development, and bone repair processes. In addition, there is strong evidence that OCP expression is tightly regulated, and induced during the earliest stages of bone fracture repair when osteo-/chondro-progenitor cells are recruited. This observation suggests that OCP plays a role in this process.

Taking into account the pattern of 608 expression during the process of bone fracture healing, it is tempting to suggest that 608-positive precursor cells are involved not only in remodeling of intact bone but also in the repair processes of the fractured bone as well.

### EXAMPLE 8

### OCP Transcriptional Regulation

In order to clone the longest possible fragment which will contain the OCP regulatory region/s, bacs L4 and H7 were restricted with three different enzymes: BamHI, Bgl II and SauIIIA. The resulting fragments were cloned into the BamHI site of pKS. Ligation mixes were transformed into bacteria (E. coli—DH5a) and 1720 colonies were plated onto nitrocellulose filters which were screened with ³²P-labeled PCR fragment spanning the mouse-OCP-exon1. Positive colonies were isolated.

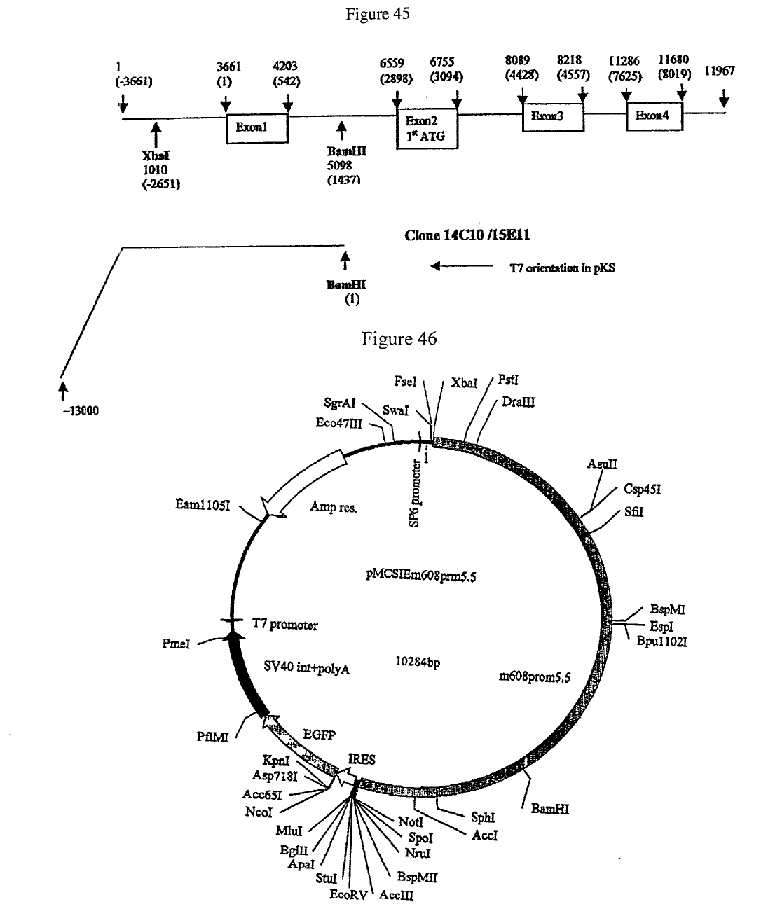

Two identical clones, 14C10 and 15E11, contained the largest inserts (BamHI derived ˜13Kb inserts). The structure of the insert compared to the “Lexicon” clone previously mentioned is illustrated in FIG. 45. The 14C10 clone is longer than the OCP “Lexicon” clone by ˜8Kb at the 5′end.

a) Cloning of Mouse OCP Promoter and UTR Upstream to the Reporter Gene—EGFP

The 1.4Kb genomic region of the mouse OCP gene, flanked by BamHI site (nuc 5098 of the “Lexicon” clone which is the start site of clone p14C10) and the first ATG codon (first nucleotide of exon 2), was synthesized by genomic PCR using the “Lexicon” clone as template and pre-designed primers: 5′primer (For1) located upstream to the BamHI site (nucleotides 4587-4611 of the Lexicon clone) and 3′ primer (Rev 2) located immediately upstream to the first ATG (nucleotides 6560-6540 of the Lexicon clone) and tailed by a NotI site. The PCR product was cut by BamHI and NotI and the resulting 1.4Kb fragment was ligated to pMCSE into BamHI/NotI sites upstream to the EGFP reporter gene. The resulting clone was designated pMCSIEm608prm1.4.

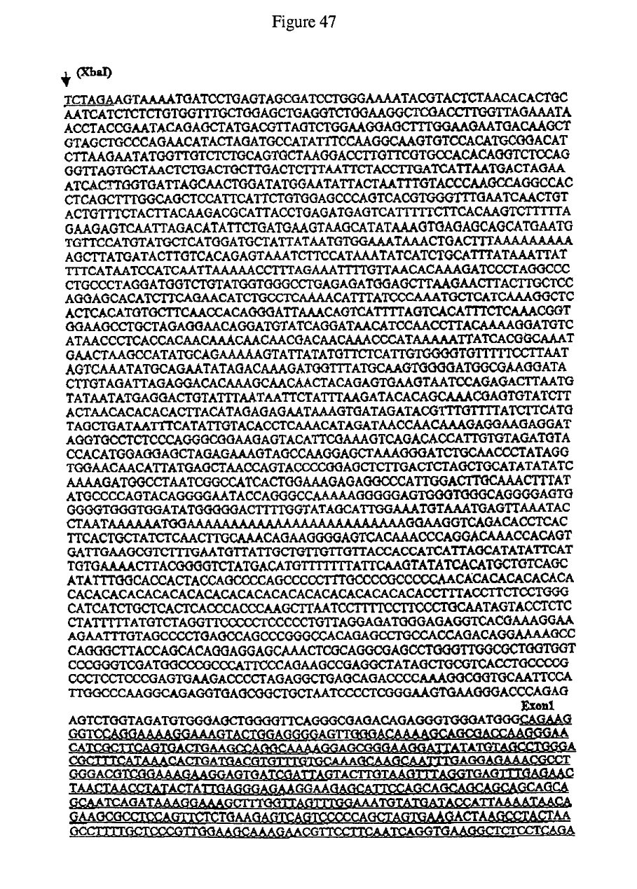

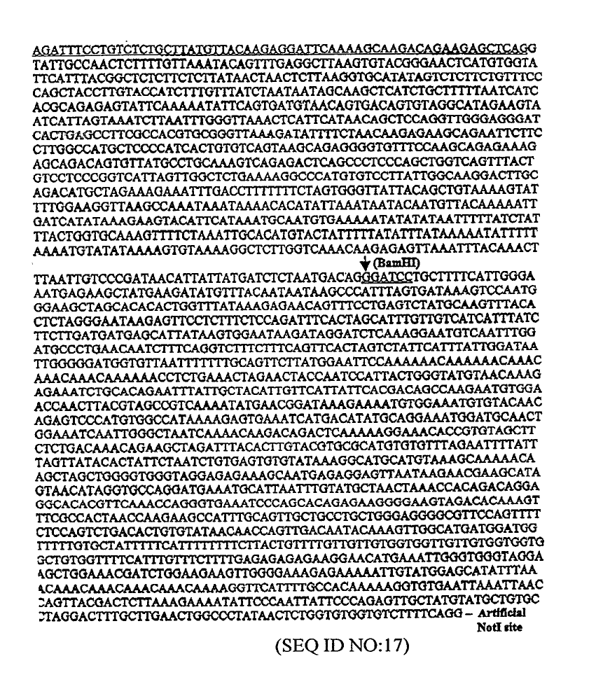

Clone p14C10 was cut by XbaI and BamHI and the excised 4.088Kb fragment was ligated into the BamHI and XbaI sites of pMCSIEm608prm1.4, upstream to the 1.4Kb insert. The resulting clone (shown in FIG. 46) was designated pMCSIEm608prm5.5 and contains 5552 nucleotides of the mouse 608 promoter and UTR upstream to EGFP. The insert of pMCSIEm608prm5.5 clone was completely sequenced, as can be seen in FIG. 47.

The whole 13Kb insert of p14C10 was excised by BamHI and ligated upstream to the 1.4Kb insert of of pMCSIEm608prm1.4 into the BamHI site. The resulting construct, pMCSIEm608prm14.5 contains a 14.5Kb fragment of the mouse-OCP promoter and UTR upstream to EGFP.

b) Transient Transfection Results

The two constructs, pMCSIEm608prm5.5 and pMCSIEm608prm14.5, were injected to fertilized mouse eggs and two weeks old transgenic and control mice were sacrificed for the detection of GFP activity in calvaria and long bones. No specific fluorescence was detected, partly because of background fluorescence from various tissues and partly because of the cellular specificity of OCP expression. Therefore, the inventors decided to use the more sensitive luciferase gene as the reporter gene.

c) Clonino Mouse OCP Promoter and UTR Upstream to the Reporter Gene-luciferase

Both inserts of pMCSIEm608prm5.5 and of pMCSIEm608prm14.5 were also cloned upstream to luciferase, in Promega's pGL3-Basic vector. The 5.5Kb insert of pMCSIEm608prm5.5 was excised by EcoRV and XbaI and ligated to SmaI and NheI sites of pGL3-Basic vector. The resulting clone is designated pGL3basicm608prm5.5.

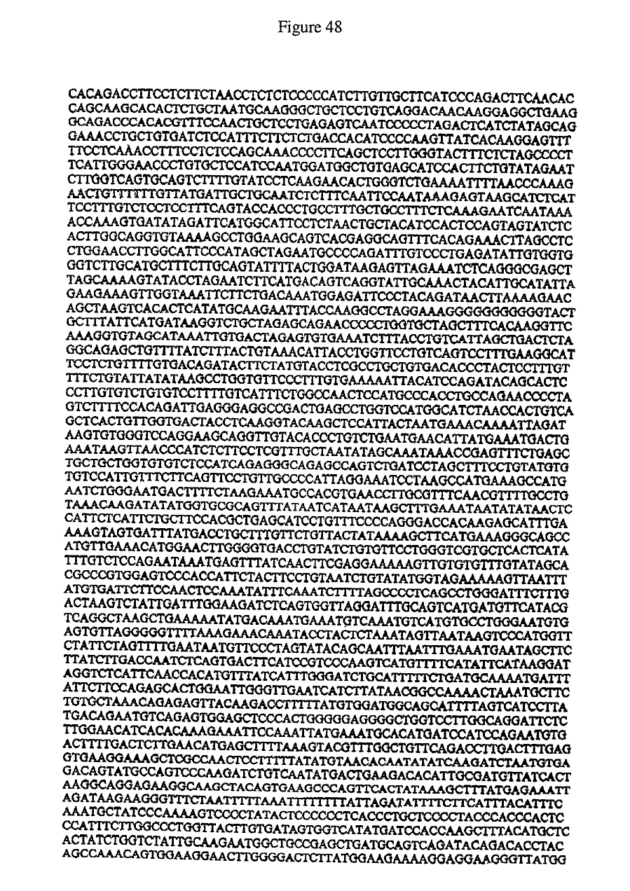

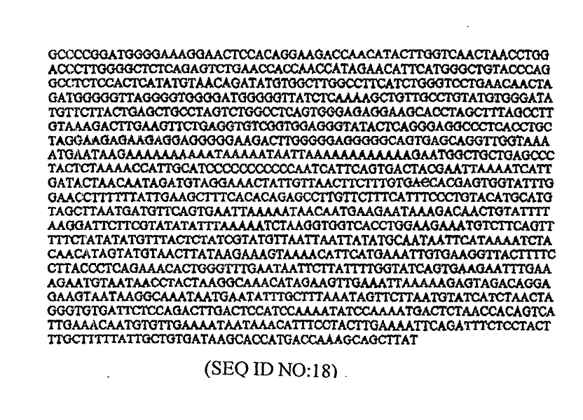

Plasmid pMCSIEm608prm14.5 was restricted by NotI and the cohesive ends of the linearized plasmid were filled and turned into blunt ends. The 14.5Kb insert was then excised by cutting the linear plasmid by SalI. The purified 14.5Kb fragment was ligated to the XhoI and HindIII (filled in) sites of pGL3-basic upstream to the luciferase gene to create the construct designated pGL3basicm608prm14.5. FIG. 48 depicts 4610 bp that have been sequenced. d) Transient Transfection Results

At this stage transient transfection of both constructs to primary calvaria cells, resulted in 10-fold expression only upon pMCSIEm608prm14.5 transfection. No enhanced promoter activity was observed upon pMCSIEm608prm5.5 transfection. These observations suggest that the region between the 5′end of pMCSIEm608prm14.5 and the 5′end of pMCSIEm608prm5.5 is necessary for full promoter activity. Further analysis is in process to detect all the sequences that are necessary and sufficient for maximal promoter activity and tissue specific OCP induction or repression in various cell systems.

e) Analysis of TF Binding DNA Elements Common to Mouse and Human OCP

Known transcription factor (TF) binding DNA elements were analyzed for similarity upstream of human and mouse OCP ATG using the DiAlign program of Genomatix GmbH. The genomic pieces used are the proprietary mouse genomic OCP and reverse complement of AC024886 92001 to 111090. The locations of the ATG in these DNA pieces are:

575 on rat cDNA

*6521 on mouse genomic

*3381 on the piece extracted from human genomic DNA AC0024886 14 elements were extracted in this procedure and analyzed for transcription binding motifs using the MatInspector.

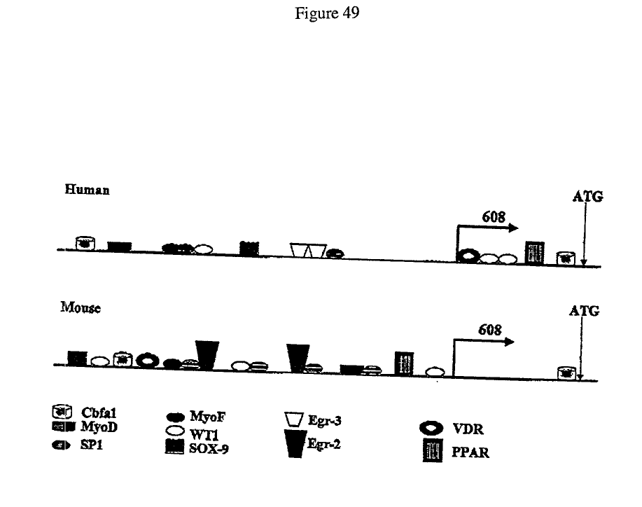

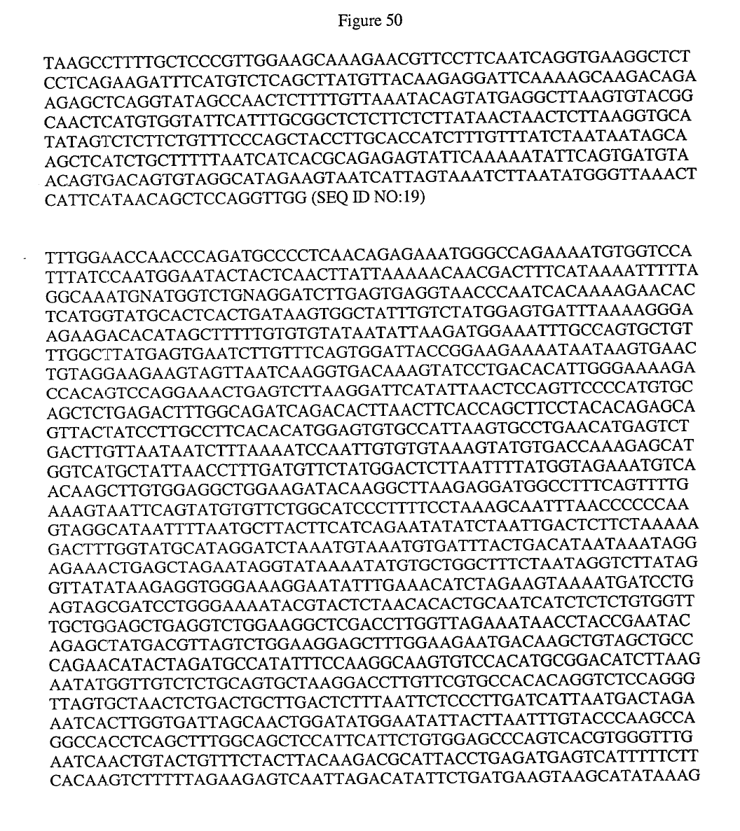

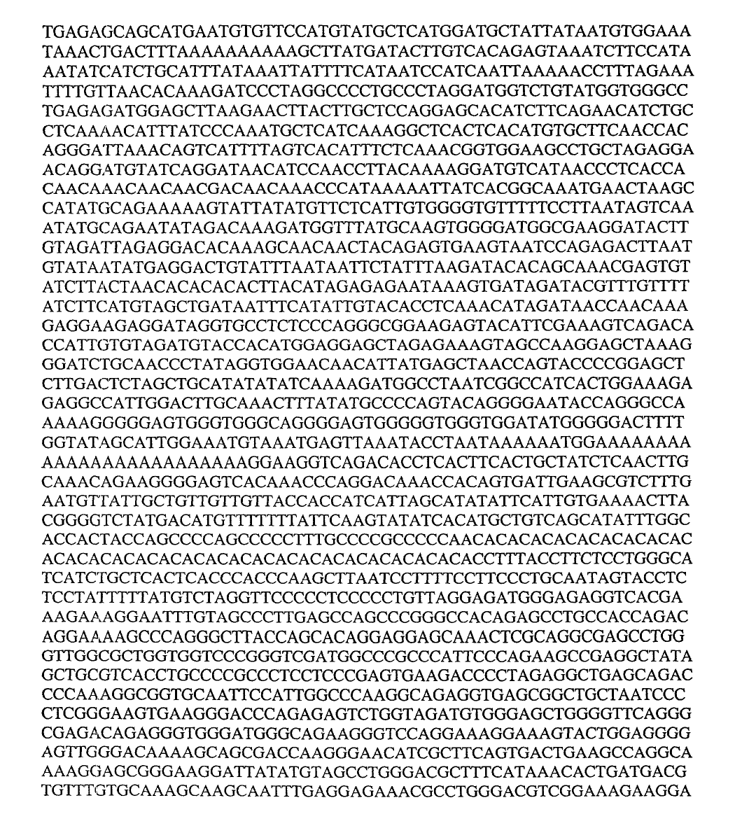

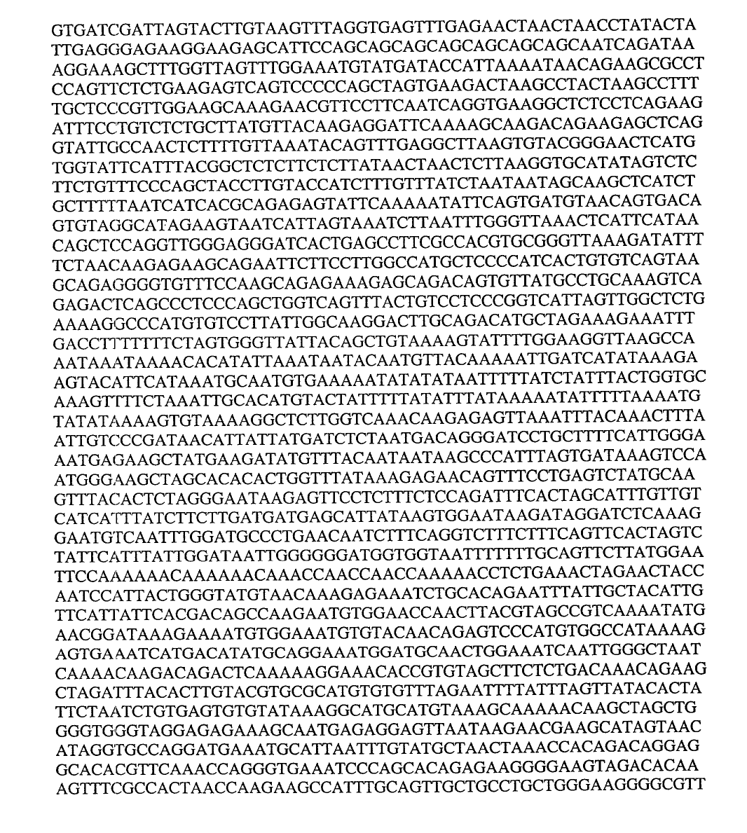

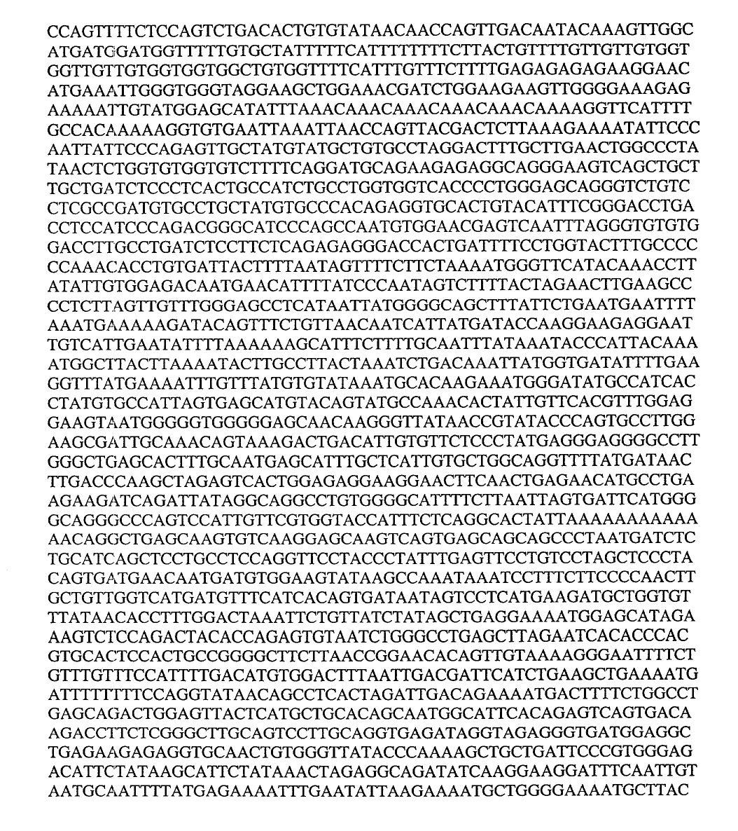

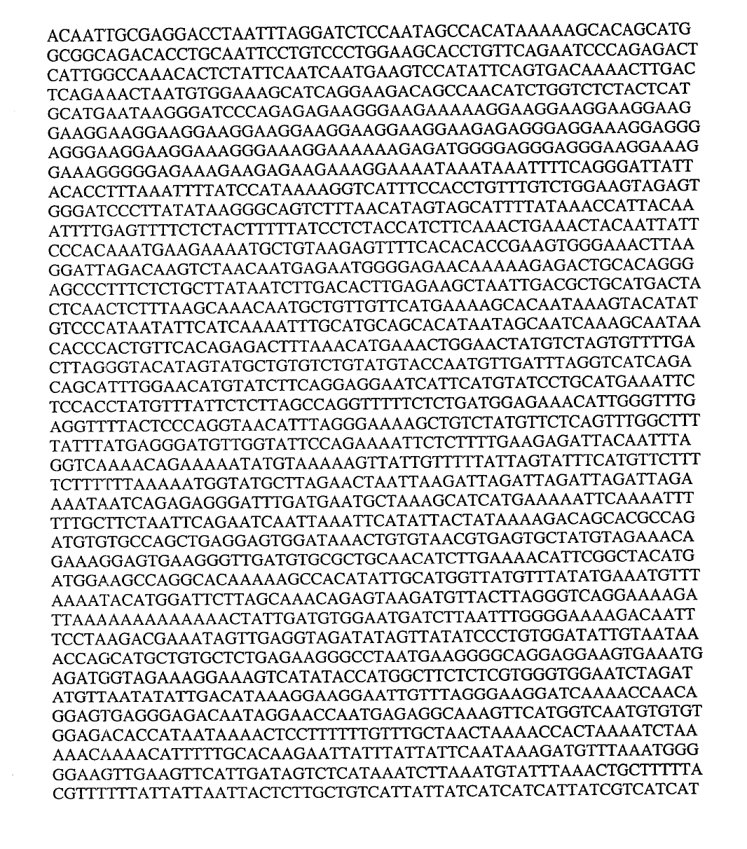

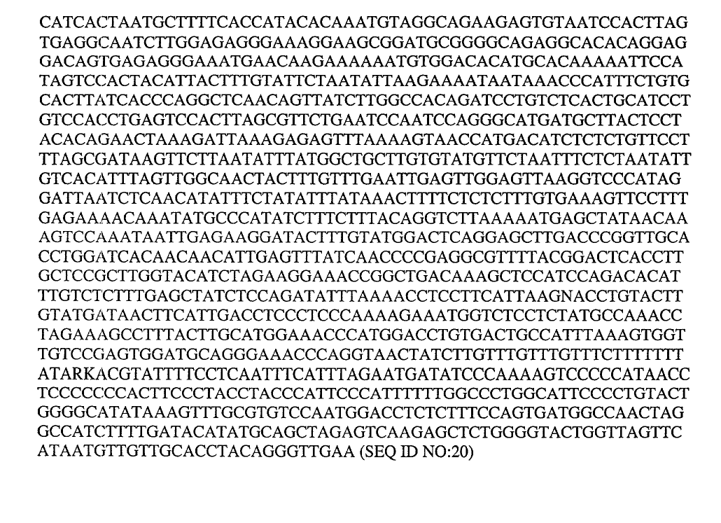

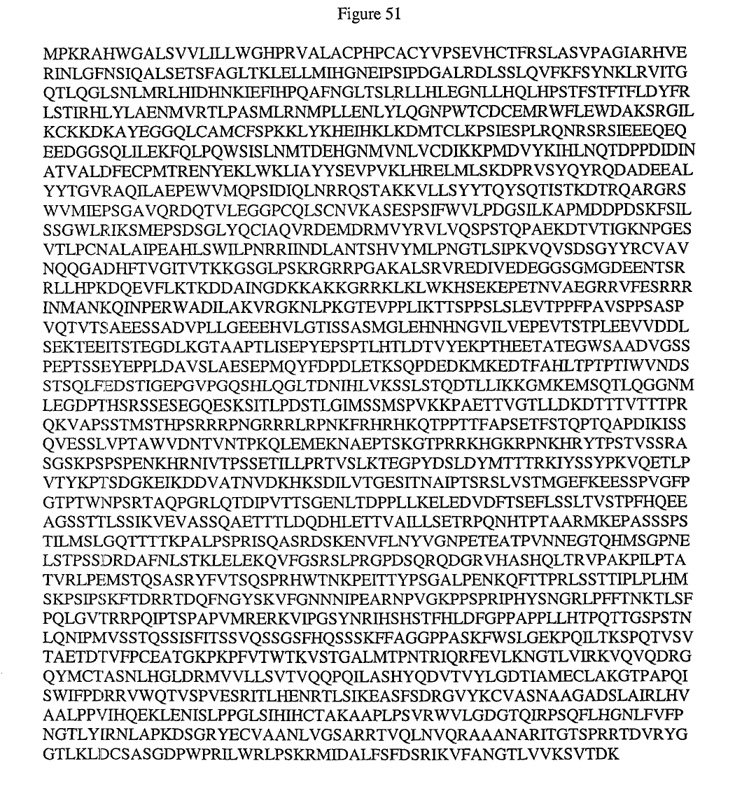

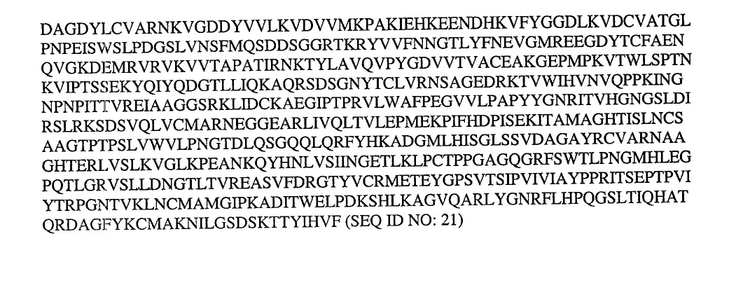

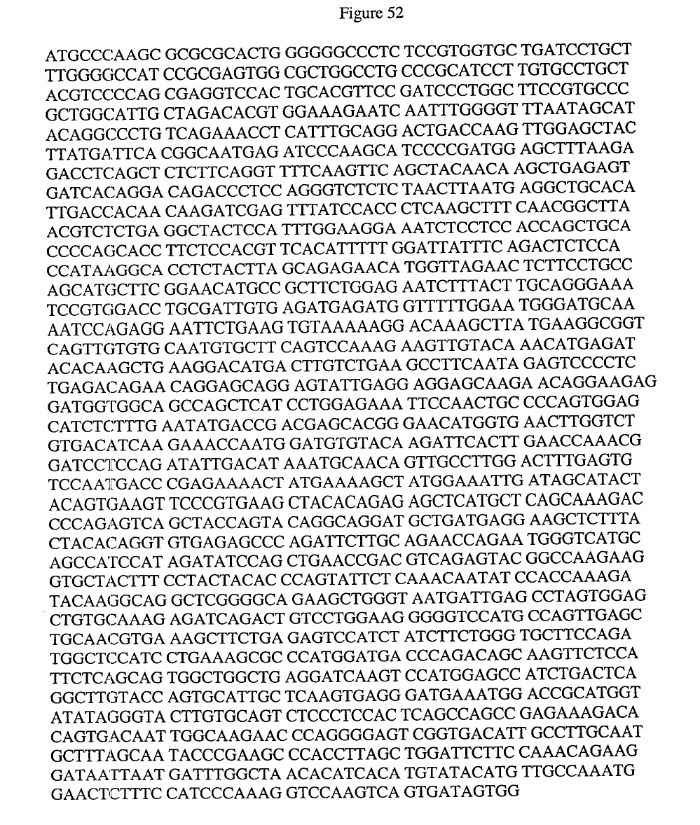

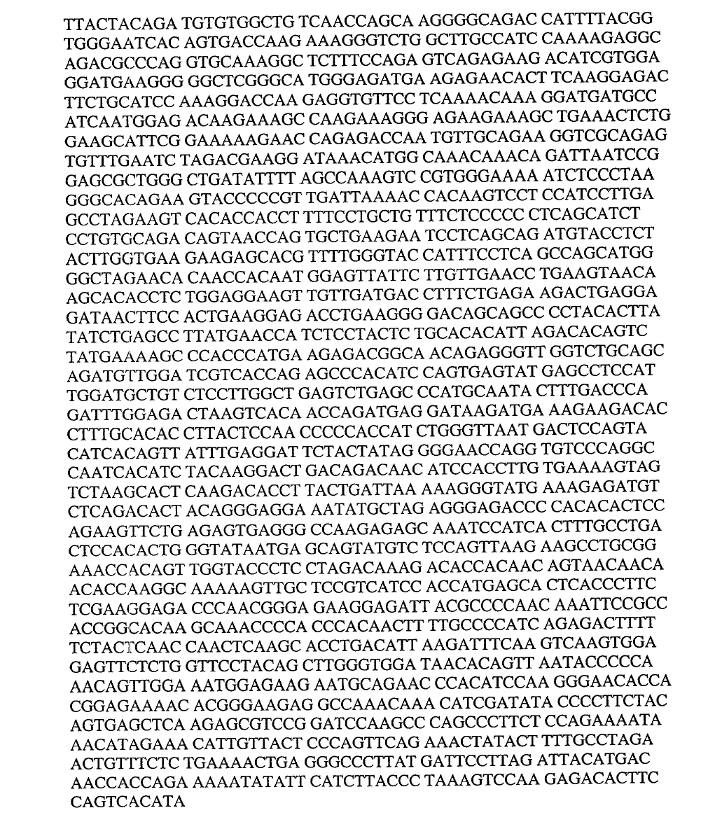

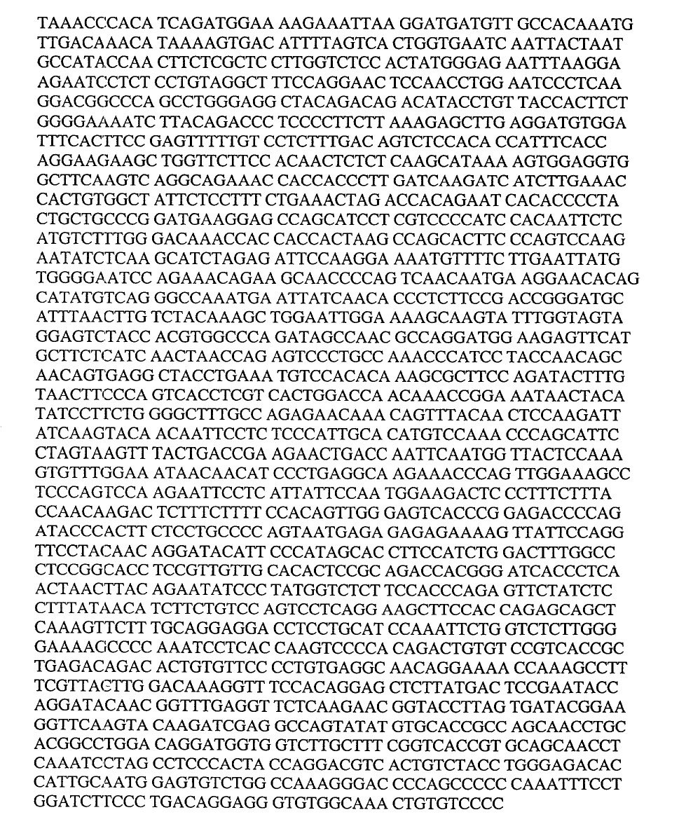

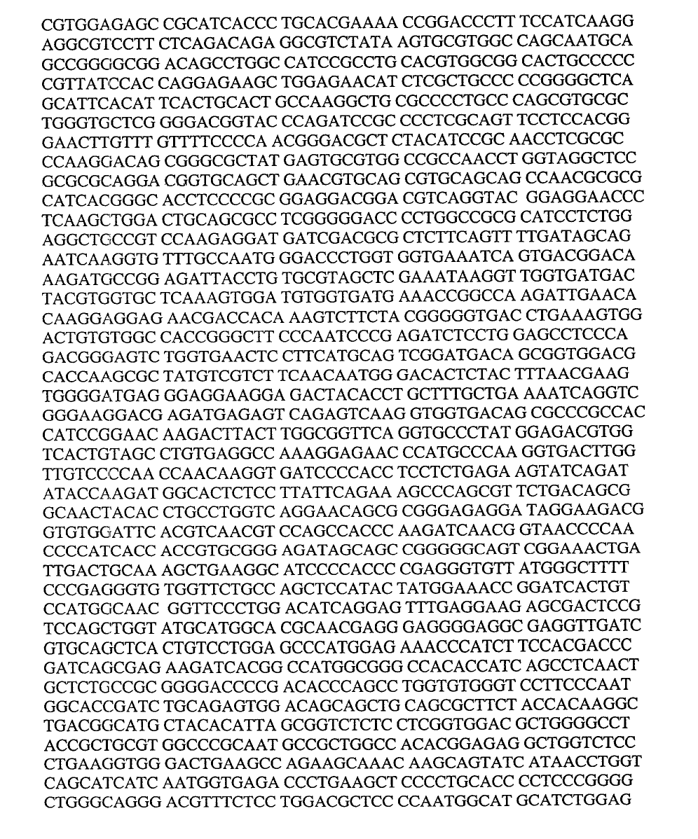

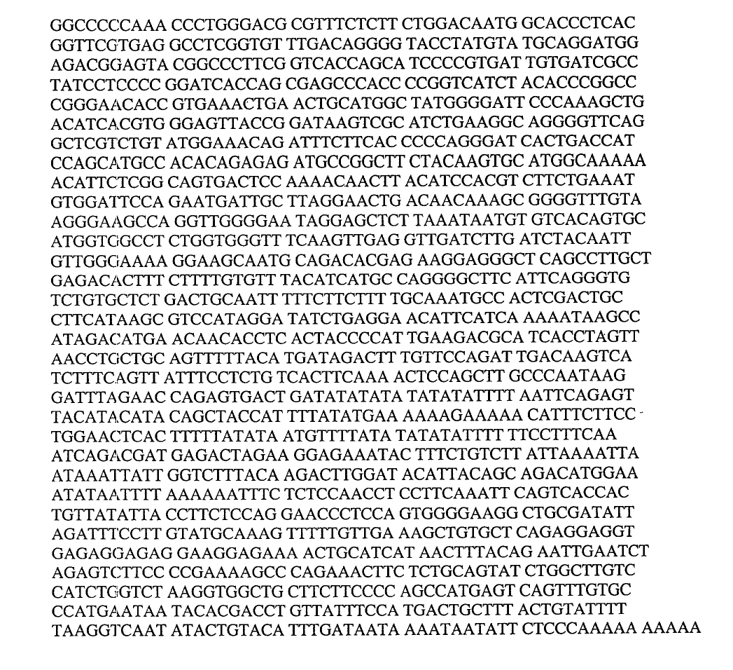

Some of the main “master gene” binding sites are illustrated in FIG. 49. Among them are the osteoblast-/chondrocyte-specific Cbfal factor; the chondrocyte-specific SOX 9 factor; the myoblast-specific Myo-D and Myo-F factors; the brain- and bone-specific WT1; Egr 3 and Egr 2 factors (Egr superfamily); the vitamin D-responsive (VDR) factor; the adipocyte-specific PPAR factor; and the ubiquitous activator SP 1.

### EXAMPLE 9

### Expression Pattern and Regulation of Gene 608 Expression of Gene 608 in Regard to other Osteogenic Lineage Markers

Expression of gene 608 was tested in primary cells and in cell lines with regard to expression of various markers of osteogenic and chondrogenic lineages. The results of this analysis are summarized in the following table and showed that expression of 608 is restricted to committed early osteoprogenitor cells.

| Cells | 608 | Collagen I | Collagen II | Alk. Phos. | Osteocalcin | Cbfa1 | Osteopontin |
|---|---|---|---|---|---|---|---|
| STO | − | − | + | − | + | + | + |
| (fibroblasts) | | | | | | | |
| ROS | − | − | − | + | + | +/− | + |
| (osteosarcoma) | | | | | | | |
| MC3T3 (pre- | + | − | − | + | + | + | + |
| osteoblasts) | | | | | | | |
| C2C12 (pre- | − | − | − | − | + | − | + |
| myoblasts) | | | | | | | |
| C6 | − | | | − | | | |
| (glioma) | | | | | | | |
| Calvaria mouse | + | | | + | | | |
| Calvaria rat | + | | | + | | | |
| C3H10T1/2 | − | − | + | − | + | − | + |
| (mesenchymal | | | | | | | |
| stem cells) | | | | | | | |

### EXAMPLE 10

### OCP Expression is Mechanically Induced in MC3T3 E1 Cells

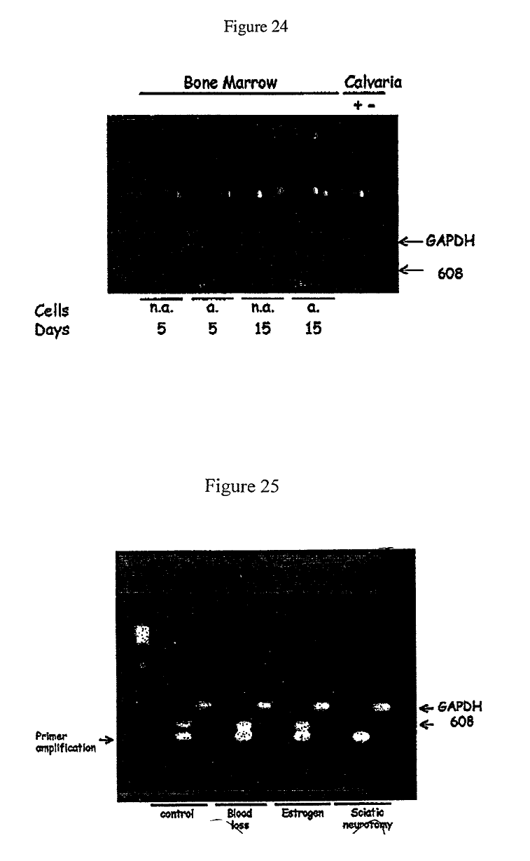

OCP transcription was detected by RT-PCR in mouse calvaria cells, U20S cells (human osteosarcoma cell line), and human embryonal bone. FIG. 24. OCP was initially discovered as being upregulated during mechanical stress in calvaria cells. In the present invention, we demonstrate that the influence of mechanical stress on OCP expression can be reproduced in another cell system using a different type of mechanical stimulation. In serum-deprived MC3T3-E1 pre-osteoblastic cells, mechanical stimulation caused by mild (287× g) centrifugation markedly induced OCP mRNA accumulation. FIG. 25. Other osteoblastic marker genes (osteopontin, ALP (staining—not shown) and Cbfal) were transcriptionally augmented by this procedure. FIG. 25. The RT-PCR product of a non-osteoblastic marker gene (GAP-DH) was used as a control to compare RNA levels between samples. No increased expression was noticed when the latter primers were used. No expression was detected in non-osteoblastic cells (FIG. 24), suggesting that OCP expression is specifically induced in osteogenesis.

### EXAMPLE 11

### OCP Induction During Endochondral Growth—in Situ Hybridization Analysis

Our previous results demonstrated that OCP is expressed during adult mice bone modeling and remodeling. The expression was restricted to the following regions:

1 perichondrium

2 periosteum

3 active remodeling and modeling regions

4 perivascular connective tissue

5 articular cartilage covering cells

6 embryo-condensed mesenchymal cells—head, vertebrae and trunk

7 ectopic bone formation

No previous observations suggest any role for OCP in bone development or initiation of endochondral ossification (longitudinal growth of long bones). Thus, the expression pattern of OCP by in situ hybridization on sections of bones from 1 week old mice was analyzed. At this stage of bone development, osteogenesis starts within the epiphysis (secondary ossification center). The hind limb skeleton of 1 week old rat pups (femur together with tibia) was fixed in buffered formalin and longitudinal sections of decalcified tissue were processed for in situ hybridization according to standard in-house protocol. Autoradiographs were developed, stained with hematoxylin-eosin and studied under microscope using brightfield and darkfield illumination.

A strong fluorescence signal was observed all over the second ossification center using OCP probes. FIG. 27. In addition, the hybridization signal delineates periosteal and perichondrial tissue in a way similar to that found earlier in adult bones. Surrounding mature chondrocytes displayed no signal. A very faint signal was observed using the osteocalcin probe which is a marker of mature osteoblasts.

In conclusion, OCP is expressed in osteoprogenitor cells that initiate eridochondral ossification during bone development.

### EXAMPLE 12

### In Vivo Regulation by Stimuli Either Promoting or Suppressing Bone Formation: Estrogen Administration, Blood Loss and Sciatic Neurotomy

Osteogenic cells are believed to derive from precursor cells present in the marrow stroma and along the bone surface. Blood loss, a condition that stimulates hemopoietic stem cells, activates osteoprogenitor cells in the bone marrow and initiates a systemic osteogenic response. High-dose estrogen administration also increases de novo medullary bone formation possibly via stimulation of generation of osteoblasts from bone marrow osteoprogenitor cells. In contrast, skeletal unweighting, whether due to space-flight, prolonged bed-rest, paralysis or cast immobilization leads to bone loss in humans and laboratory animal models. To detect alteration in OCP expression pattern following the above procedures, the following experiments were performed on two month old mice:

estrogen administration (500 μg/animal/week),

bleeding (withdrawing approximately 1.6% body weight),

unilateral (right limb) sciatic neurotomy,

control groups for each treatment

Total RNA was extracted from long bones after two-day treatment and RT-PCR using OCP-specific primers was performed. The results demonstrate that OCP expression was highly enhanced following blood loss and estrogen administration, while down-regulation was observed following sciatic neurotomy. FIG. 29.

By having a unique cell marker (OCP) we can show that the above procedures induce or reduce bone formation by increasing or decreasing the number of osteoprogenitor cells. The above results suggest once more that OCP is a major member of a group of “bone specific genes” that regulate the accumulation of bone specific precursor cells.

### EXAMPLE 13

### OCP Induction During Osteoblastic Differentiation of Bone Marrow Stroma Cells

Bone formation should be augmented in trabecular bone and cortical bone in osteoporotic patients. We have previously detected OCP expression in periosteum and endosteum (surrounding the cortical bone) but no signal was apparent in bone marrow cells. The latter cells normally differentiate to mature osteoblasts embedded in the trabecular and cortical bone matrix.

To further assess OCP expression in bone marrow progenitor cells, the inventors extracted total RNA from mouse and rat bone marrow immediately after obtaining it and after cultivation for up to 15 days in culture. No OCP-specific RT-PCR product was detected with RNA from freshly obtained bone marrow (both in adherent and non-adherent) cells. However, a faint signal was found after 5 days in culture, and it was further enhanced when RNA from cells grown for 15 days in culture was used. ALP (alkaline phosphatase) expression (an osteoblastic marker) was also found to be enhanced after 15 days. At both time points, adherent and non-adherent cells were reseeded, and RNA extractions were prepared 5 and 15 days later. A stronger RT-PCR product was observed with RNA extracted from originally adherent cells, suggesting the existence of less mature progenitors in the non-adherent population of bone marrow cells. The RT-PCR product of a non-osteoblastic marker gene (GAP-DH) was used as a control to compare RNA levels between samples.

In conclusion, bone marrow progenitor cells do not express OCP, but differentiate to more committed cells that do express this gene.

### EXAMPLE 14

### OCP Induction During Mesenchymal Cell Differentiation Towards Osteogenesis

Mesenchymal stem cells (MSC) are multipotent, self-renewing cell populations which undergo differentiation and commitment to give rise to monopotent cells of specified lineages, such as osteoblasts. The mechanisms of commitment and self-renewal are not fully understood, but may be regulated by factors such as Bone Morphogenetic Proteins (BMPs), differentiation factors such as retinoic acid (RA) and steroid hormones such as glucocorticoids. Furthermore, BMP and RA act synergistically to stimulate osteoblastic commitment and cell proliferation.

In order to find out if OCP expression is induced upon osteoblastic commitment, quiescent C3H10T½ murine MSC cultures were stimulated with BMP and RA for 24 hours and cultured in full medium for further 3 days. RNA was extracted from non-treated cells as well as from cells harvested at 24hrs, 48hrs and 72hrs after the beginning of the treatment, and used for RT-PCR analysis with OCP-specific primers. In parallel, cells were stained for ALP to determine osteoblastic commitment. While ALP staining was apparent only on day 3 (72hrs), OCP expression was augmented by day 1 (24hrs), being undetectable in non-treated cultures. Further experiments have shown that even stronger ALP staining and OCP expression were observed on day 6 following proliferation and further differentiation of osteoprogenitor cells. FIG. 26.

These results demonstrate that upon osteoblastic commitment of MSCs, OCP expression is “switched-on” before the commencement of ALP expression, suggesting that this candidate is the earliest marker gene of osteoprogeniter cells found to date. It should be noted that our previous in situ hybridization results also demonstrated the presence of OCP transcripts only in very early chondro-/osteo-progenitor cells. These cells did not express ALP, and more mature ALP positive cells (in the trabecular bone) were OCP negative.

### EXAMPLE 15

### OCP Induction During Differentiation Switch of Pre-myoblasts to Osteoblasts

Pre-myoblastic cells (C2C12) give rise to mature myoblasts. As with C3H10T½, the administration of BMP and RA to these cells can induce osteoblastic differentiation. To investigate the expression pattern of OCP during this differential switch we introduced BMP and RA to C2C12 cells and analyzed cell fate and expression pattern as above (for C3H10T½ cells). As expected OCP and ALP expression were induced 24hrs post-BMP introduction. FIG. 26.

These assays once more demonstrate the involvement of OCP in the early stages of osteogenesis.

### EXAMPLE 16

### OCP Role in Osteogenesis

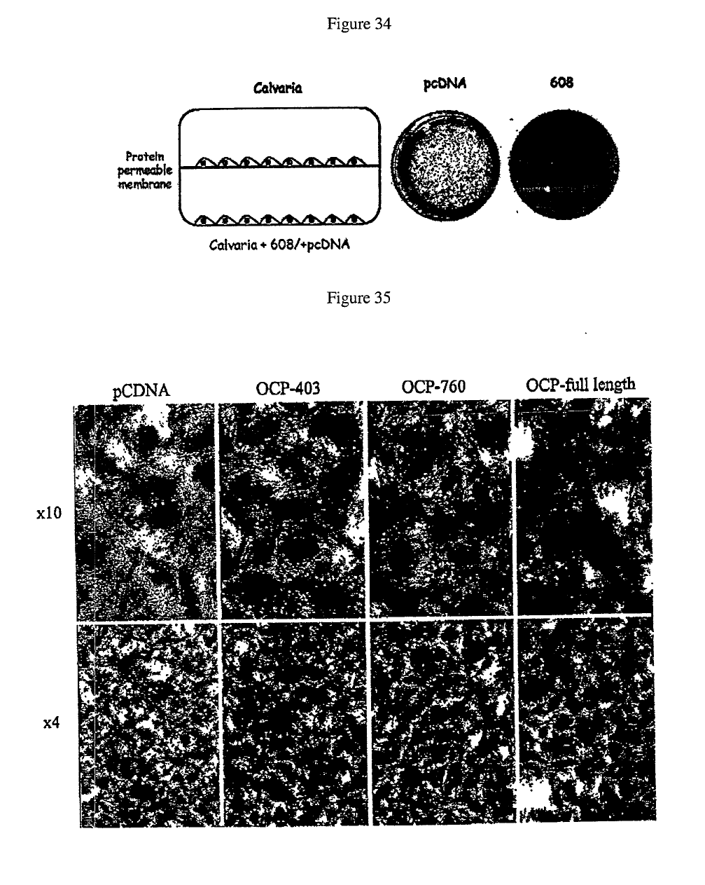

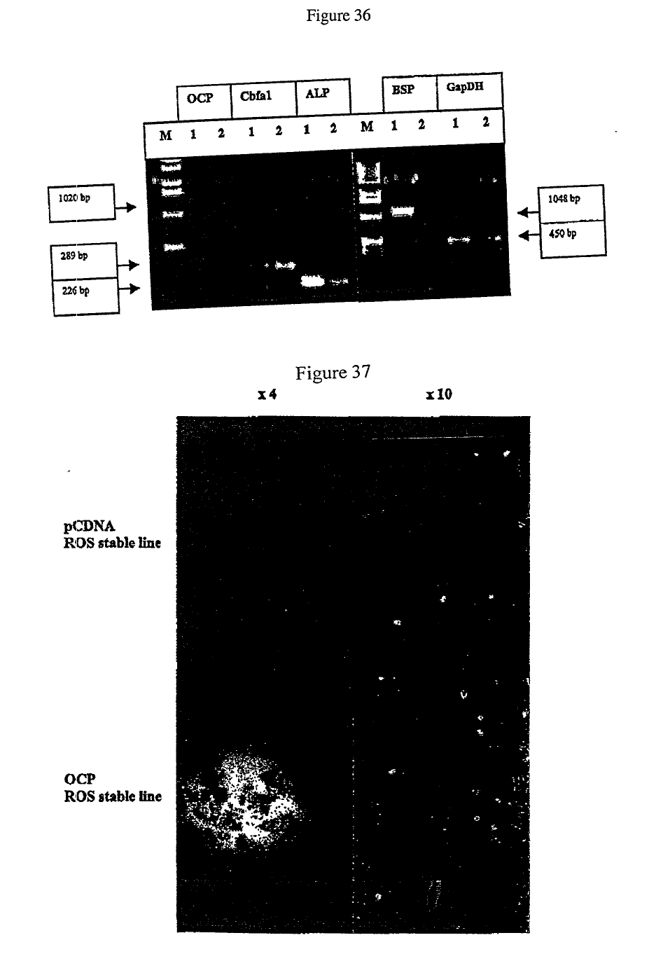

The ultimate test for the role of OCP as a crucial factor that induces osteoblast-related genes is its ability to up-regulate these genes in pre-osteoblastic and osteoblastic cells. In primary calvaria cells, transient transfection with a CMV promoter-driven OCP construct significantly up-regulated the expression of the osteogenic lineage marker ALP. FIG. 34 illustrates the induction in ALP staining. Transient transfections of two smaller deletion constructs of the OCP gene also gave the same induction (FIG. 35), suggesting that the N-terminal 403 amino acid protein stretch (which contains a signal peptide) is necessary and sufficient to augment osteoblastic proliferation and differentiation. In addition, stable transfection of OCP to ROS 17/2.8 (differentiating osteoblast cell line) cells, also substantially upregulated ALP and BSP expression, while repressing Cbfa1 transcription (Cbfa1 is known to be expressed early in the osteoblast lineage and to be transcriptionally downregulated during cellular aging of osteoblasts). FIG. 36. In addition, marked increase in osteoblastic proliferation was observed. FIG. 37.

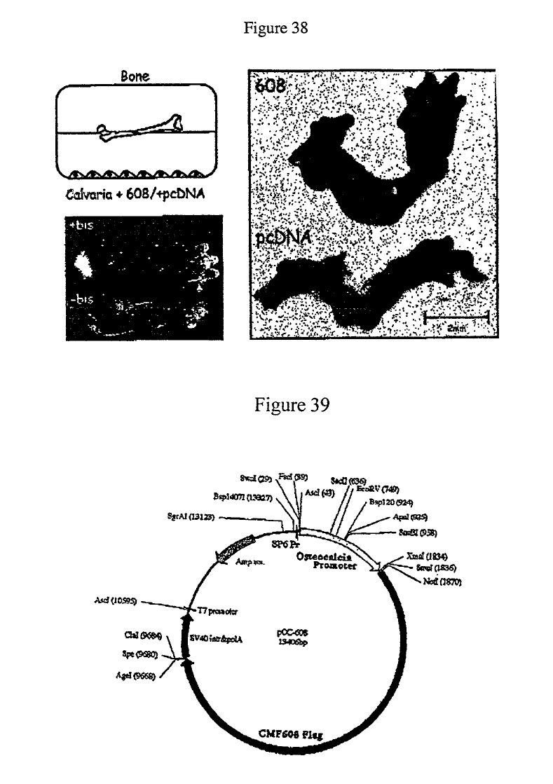

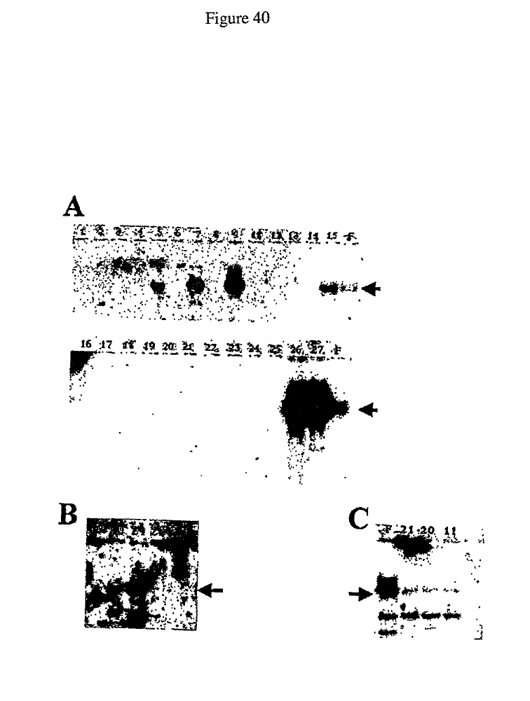

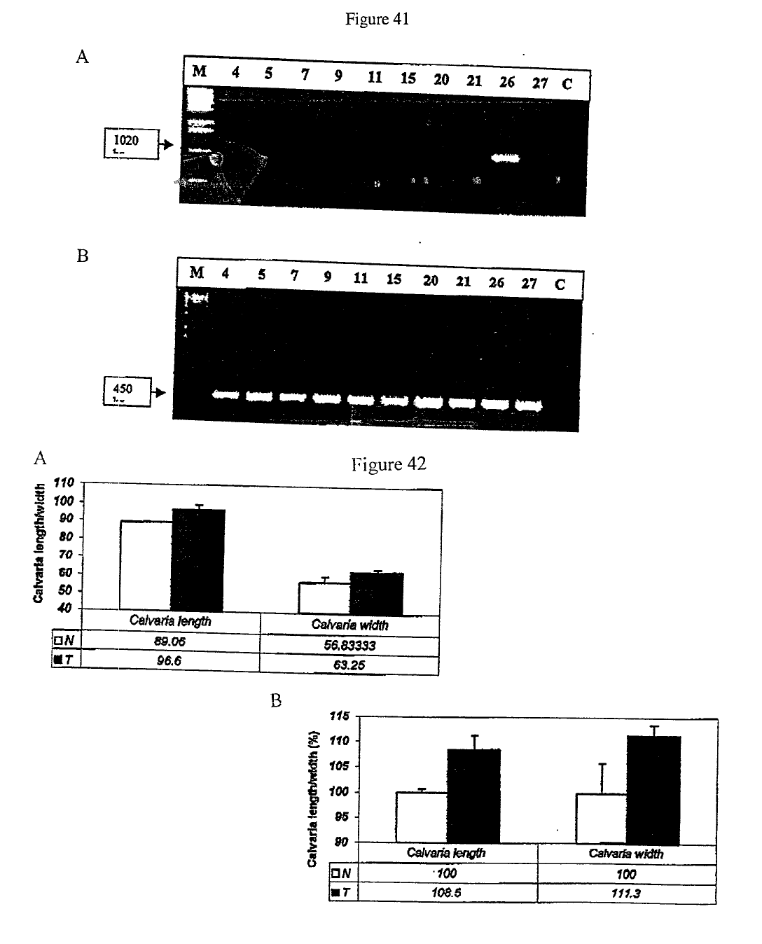

Further experiments have shown that the osteogenic effect of OCP expression in calvaria cells is non-cellautonomous. In a co-cultivation assay where OCP-transfected calvaria cells were cultured in the presence of nontransfected calvaria cells (that were grown on a millipore filter), the osteogenic induction effect was also evident as was illustrated in FIG. 38. The non-transfected cells that were cultured in the presence of C)CP-transfected cells retained elevated ALP activity compared to control assays. No similar effects were observed upon transfection to the pluripotent progenitor C3H10T½ cells that can differentiate to myoblasts, osteoblasts, adipocytes or chondrocytes or to C2C12 pre-myoblast cells.

These results provide compelling evidence that OCP is an essential factor required for the initiation of the signaling cascade that leads to sequential expression of other phenotype-specific genes committed to the osteogenic lineage. In addition, these results support accumulation of an OCP-dependent osteogenesis factor that seems to act as a secreted factor. We have no data yet as to whether this factor is the OCP product or an OCP-induced factor.

### EXAMPLE 17

### Bone Culture Assays

To further confirm the involvement of OCP in bone formation, we performed organ culture of E16 mouse embryonal limbs. The limb bones were stained with Alizarin Red following 6 days of culture to compare bone calcification rate. When the E16 mouse embryonal limbs were cocultivated with OCP-transfected calvaria cells, both endochondral and membranous ossification were enhanced as illustrated in FIG. 42. In contrast to the control limbs (cocultivated with vector-transfected calvaria cells), the OCP transfection to calvaria cells resulted in the formation of bones that are longer and wider in their proximal and distal extremities. Thus, we have shown that the osteogenic inducing effect of OCP that was observed in vitro, can be also demonstrated ex vivo by the induction of bone formation in cartilage bone rudiments. The role of OCP in bone rudiments probably mimics its role in endochondral ossification and bone development of mouse fetuses.

### EXAMPLE 18

### Oc-OCP Transgenic Mice

To verify the results presented in the present invention, the inventors generated transgenic mice in which 608 expression is induced in mature osteoblasts by coupling the OCP cDNA to the osteocalcin (Oc) promoter. Construction of the pOC-608 vector.

The Oc promoter was amplified using primers according to the literature. The promoter was taken from plasmid pSROCAT (Lian et al. (1989) Proc. Natl. Acad. Sci. U.S.A. 86:1143-1147) using SmaI and HindIII (blunted) and sub-cloned into the blunted BamHI and XbaI sites of the vector pMCS-SV producing the vector pOC-NSV.

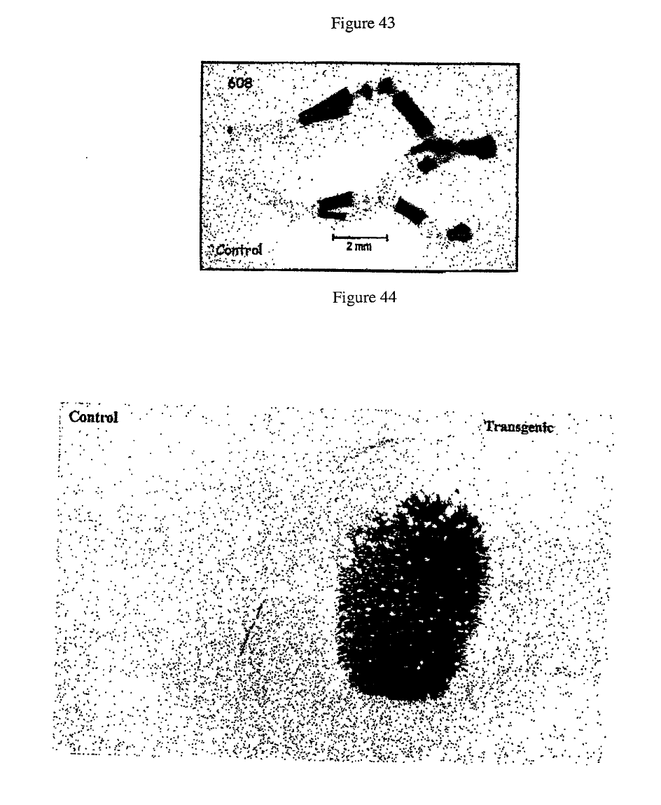

The CMF608 Flag fragment was isolated from the pCDA3.1-608 construct (FIG. 2) after NotI and SpeI digest. The fragment was sub-cloned into the NotI-SpeI sites of the pOC-MCS vector. The construct was verified by extensive sequencing. FIG. 43.

Preparation of DNA for Microinjection

For the preparation of the DNA insert for microinjection the plasmid was digested with AscI (cuts at bp 43 and bp 10595). The ˜10.6Kb fragment was isolated from agarose gel using the Qiaex II kit (Qiagen Cat No. 20021) and then purified over an Elutip-D column (Schleicher & Schuell Cat. No. NA010/1).

Derivation of Transgenic Mice

The DNA was dissolved in a pure Tris/EDTA microinjection solution and adjusted to a concentration of 2 ng/μl. Standard pronuclear microinjection into fertilized eggs from the FVB/N strain and embryo transfer into ICR foster mothers was performed as described in the literature. See, Manipulating the Mouse Embryo, Hogan, Beddington, Constantini and Lacy, Cold Spring Harbor Laboratory Press.

Embryo Recovery

Foster mothers were sacrificed by cervical dislocation at day 18 post-embryo transfer. Embryos were recovered and placentas were taken for DNA preparation and analysis of the presence of the injected OC608-Flag DNA in the mouse genome.

Genomic DNA Analysis

Mouse genomic DNA was recovered from the placenta using standard procedures. Laired et al. (1991) Nucl. Acids Res. 19:4293. Genomic DNA was digested with EcoRV, separated on 1% agarose gel and blotted onto Nytran nylon membranes (Schleicher & Schuell). The blots were hybridized with a SV40 intr&polA labeled probe (see map) overnight and washed the following day. Membranes were exposed to X-ray film and developed after 24 and 48 hours. FIG. 44.

OCP Exogenic RNA Expression Analysis

To determine which of the transgenic embryos expressed the exogenic OCP, total RNA was isolated from the hind legs according to the manufacturer's instructions (EZ-RNA, total RNA isolation kit, Biological industries). 5 μg of total RNA was assayed by RT-PCR according to the manufacturer's instructions (GIBCO BRL SuperScript™ II). As, a negative control, RT was omitted. PCR was performed for 30 cycles (1 min at 94° C., 1 min at 59° C.,and 2 min at 72° C.), using Taq polymerase (Promega) and either exogenic OCP or GapDH primers that amplify cDNA products of 1020 bp and 450 bp, respectively.

The following primers were used for exogenic OCP detection:

Forward: 5′ GCACTGAACTGCTCTGTGGAT 3′ (SEQ ID NO:22); and

Reverse: 5′ CCACAGAAGTAAGGTTCCTTCAC 3′ (SEQ ID NO:23).

Reaction products (5 μl per lane) were electrophoresed in 1.5% agarose and stained in ethidium bromide. As illustrated in FIG. 45, similar amounts of GapDH transcripts were detected in all RNA samples from all tested embryos, indicating that differences in OCP transcript abundance did not reflect variation in the efficiency of the RT reaction. In addition, no GapDH PCR products were detected in any RNA samples when RT was omitted. The results show that OCP was expressed by osteoblasts under osteocalcin promoter transcriptional regulation only in embryo numbers 5, 7, 9, 11, 15, 21, 26 and 27. FIG. 45.

Characterization of Bone Growth in Osteocalcin Promoter—608 Transgenic Embryos

The results illustrated in FIGS. 46-48, suggest that over-expression of OCP during mice embryonal development (E17) results in increased endochondral (longitudinal) and membranous ossification of long bones and increased membranous ossification of calvaria flat bones. Summarizing the above results shows that this phenotype is caused primarily by a profound increase in osteoblastic proliferation, differentiation and finally osteoblast activity.

### EXAMPLE 19

Creation of a Readout System

A readout system is created to identify small molecules that can either activate or inactivate OCP bone-precursor-specific promoter.

### EXAMPLE 20

Bioinformatic Analysis of Human 608

A DNA sequence encoding a fragment of human OCP named AC024886 is found in htgs database but not in nt. There is no genomic DNA corresponding to the rat cDNA. Alignment of AC024886 against the rat cDNA using BLAST shows two areas of long alignment (and several shorter areas):

1. cDNA: 6462-8186

Genomic: 89228-90952 plus/plus orientation: 81% identity

2. cDNA: 5581-6451

Genomic: 107710-106840 plus/minus orientation: 80% identity

Thus AC024886 is wrongly assembled in the region upstream of position 6462 (according to the rat cDNA), it is in the incorrect orientation. Using the incorrect orientation provides incorrect coding sequence and does not yield the human OCP protein.

The Genbank report on AC024886 is as follows:

LOCUS AC024886 175319 bp DNA
HTG 06-SEP-2000
DEFINITION Homo sapiens chromosome 3 clone RP11-25K24,
WORKING DRAFT
SEQUENCE, 9 unordered pieces.
ACCESSION AC024886
VERSION AC024886.10 GI:9438330
KEYWORDS HTG; HTGS₋PHASE1; HTGS₋DRAFT.
SOURCE human.
* NOTE: This is a ‘working draft’ sequence. It currently consists of 9 contigs. The true order of the pieces is not known and their order in this sequence record is arbitrary. Gaps between the contigs are represented as runs of N, but the exact sizes of the gaps are unknown. This record will be updated with the finished sequence as soon as
# it is available and the accession number will be preserved.
* 1 62523: contig of 62523 bp in length
* 62524 62623: gap of unknown length
* 62624 85445: contig of 22822 bp in length
* 85446 85545: gap of unknown length
* 85546 106059: contig of 20514 bp in length
* 106060 106159: gap of unknown length
* 106160 127908: contig of 21749 bp in length
* 127909 128008: gap of unknown length
* 128009 143068: contig of 15060 bp in length
* 143069 143168: gap of unknown length
* 143169 158734: contig of 15566 bp in length
* 158735 158834: gap of unknown length
* 158835 170042: contig of 11208 bp in length
* 170043 170142: gap of unknown length
* 170143 173715: contig of 3573 bp in length
* 173716 173815: gap of unknown length
* 173816 175319: contig of 1504 bp in length.

a. Mapping Human Genomic 608 Exons

Ten exons were mapped on the rat cDNA sequence from base 107 to 6451. Thus the first exon on the human genomic piece may be lacking. The human genomic piece (AC024886) upstream (19090 bases) of base 6462 of cDNA (reverse complement from base of AC024886 92001 to 111090) was compared with the rat cDNA using the program ExonMapper of Genomatix. In the Table, base 1 is actually 1131 in the genomic piece used so that the actual genomic location starts at 91870.

Two additional exons were mapped on the rat cDNA sequence from base 6462 to 8883. Thus bases 6452-6461 are lacking. The human genomic piece used is from base 165,337 to 175667 (10,341 bases). The same type of program was used to compare this sequence to the QBI genomic mouse 608.

Connecting the exon/intron borders from the genomic sequences yielded the predicted human and mouse cDNAs. The mouse and human predicted cDNAs were modified in order to allow frame shifts that allow a good multiple alignment of the human, mouse and rat proteins. Alignment was done using CLUSTALX and Pretty.

The cDNA modifications after the alignment of human cDNA to rat cDNA by GeneWise were as follows. In the following two tables, −x indicates a deletion of nucleotide x in the cDNA sequence; +x indicates an insertion of nucleotide x in the cDNA sequence; and all changed positions are in relation to the original sequence

| Position | Change |
|---|---|
| 1111 | −g |
| 4154 | −c |
| 4538 | +g |
| 4730 | −a |
| 4744-5 | −aa |
| 4830 | +c |
| 4852 | −g |
| 4902 | +t |
| 4942 | +c |
| 5370 | +t |
| 5387 | −a |
| 5395 | +c |

The corrections of frams-shifts in mouse 608 were as follows:

| Position | Change |
|---|---|
| 678 | −c |
| 1106 | −a |

Chromosomal Location on the Human Chromosome:

Two different types of data exist.

Genomic piece AC024886 has identity to the fragment identified as ACCESSION D14436 as described by Fukui et al. (1994) Biochem. Biophys. Res. Commun. 201:894-901.

Alignment information:

Identities=315/335 (94%),

hrh1:4 −338

AC024886-41328

Hrh1 is mapped to chromosome 3 and to 3p25; and

b. Identity to STS at 3q. STS: 20-432 is identified as ACCESSION G54370 and described by Joensuu et al. (2000) Genomics 63:409-416.

### EXAMPLE 21

### Polyclonal Antibody Preparation

Polyclonal antibodies specific to the whole 608 putative protein are prepared by methods well-known in the art (the structure of 608 resembles that of growth factor precursors). Polyclonal antibodies are identified and the recombinant active form of 608 is prepared. The activities of the polyclonal antibodies are tested in vivo in mice. The antibodies can be used for the identification of the active form of this protein which is likely to constitute a fraction of the 608 protein.

### EXAMPLE 22

### Stretch of Basic Amino Acids Found at the Boundary of the Rat and Human 608 Proteins, and its Implications

The homology between the rat and human N-terminal portions of the 608 protein is especially significant within the first 250 amino acids.

At the boundary of this conserved region there is a completely conserved stretch of basic amino acids: KCKKDR (aa 242-247 and 240-245, in rat and human proteins, respectively). Stretches of basic amino acids frequently serve as protease cleavage sites. The fact that such a stretch is found on the boundary of more or less conserved sequences and the fact that it occurs within the C-terminal LRR, a generally conserved domain, suggests an underlying biological significance.

Accordingly, the 608 protein may undergo post-translational processing through the cleavage of its highly conserved N-terminal portion and this portion may be an active part of the 608 protein or possess at least part of its biological activities. Since the resulting ˜25 kD protein preserves the signal peptide, it would be secreted.

To test whether the 25 kD cleavage product of the 608 protein is responsible for the osteogenic activity of medium conditioned by 608-transfected calvaria cells, we constructed a pcDNA vector containing the N-terminal portion of rat 608 cDNA encoding amino acids 1-241 (not including the KCKKDR stretch) and transiently expressed it in rat calvaria cells. The transfected cells were assayed for inducing bone formation both in co-cultured non-transfected calvaria cells and in ex vivo cultured E16 mouse embryo (as described above). The results clearly indicated that the secreted N-terminal portion of OCP was sufficient to stimulate osteogenesis in co-cultured cells and embryo bones.

The biologically active 25 kD N-terminal cleavage product of 608 can thus be used for treatment and/or prevention of osteoporosis, fracture healing, bone elongation and periodontosis. As an indirect product (inhibition by either chemicals or by neutralizing mAbs), the fragment can be used for treatment and/or prevention of osteoarthritis, osteopetrosis, and osteosclerosis.

### EXAMPLE 23

### The Adlican Protein and Gene

Adlican is a recently described protein. Crowl and Luk (2000) Arthritis Biol. Res. Adlican, a proteoglycan, was derived from placenta. The full amino acid sequence of Adlican is disclosed and identified as AF245505.1:1.8487, and is hereby incorporated by reference into this application. FIG. 51.

The structure of Adlican was analyzed using methods described herein and found to have leucine-rich repeats and immunoglobulin regions similar to those of the OCP protein. The overall homology found between the amino acid residues of the indicated regions in the two proteins, is as follows:

| OCP | Adlican | % |
|---|---|---|
| 1-661 | 1-669 | 38.4 |
| 662-1629 | 670-1865 | 19.7 |
| 1630-2587 | 1866-2828 | 46.5 |
| 1-2587 | 1-2828 | 33.2 |

The invention therefor encompasses the use of Adlican in any manner described herein for the OCP protein. These functions and uses have not been disclosed previously for Adlican. They include use of Adlican, or a functional portion thereof, for preventing, treating or controlling osteoporosis, or for fracture healing, bone elongation or treatment of osteopenia, periodontosis, bone fractures or low bone density or other factors causing or contributing to osteoporosis or symptoms thereof or other conditions involving mechanical stress or lack thereof in a subject. As an indirect product (inhibition by either chemicals or by neutralizing mAbs), Adlican can be used for treatment and/or prevention of osteoarthritis, osteopetrosis, and osteosclerosis.

The Adlican gene, or functional portions thereof, can likewise be used for any purpose described herein for an OCP gene. Compositions comprising the Adlican gene, Adlican or antibodies specific for Adlican and physiologically acceptable excipients are likewise encompassed by the invention. Such excipients are known in the art and include saline, phosphate buffered saline and Ringer's solutions.

Having thus described in detail preferred embodiments of the present invention, it is to be understood that the invention defined by the appended claims is not to be limited by particular details set forth in the above description as many apparent variations thereof are possible without departing from the spirit or scope thereof.

1. A composition comprising an isolated polypeptide, wherein the polypeptide is identified as Adlican or a functional portion thereof or a polypeptide which is at least substantially homologous or identical thereto and a physiologically acceptable excipient.

2. The composition according to claim 1, wherein the polypeptide is a human Adlican or a functional portion thereof.

3. The composition according to claim 2, wherein the functional portion comprises a polypeptide having a molecular weight of 10 kD to 30 kD.

4. The isolated polypeptide according to claim 1, wherein the the functional portion comprises a polypeptide having a molecular weight of about 25 kD.

5. A composition comprising one or more isolated polypeptides according to claim 1.

6. An antibody elicited by a polypeptide according to claim 1 or a functional portion thereof.

7. A composition comprising the antibody according to claim 6 or a functional portion thereof.

8. A method for treating or preventing osteoporosis, or for fracture healing, bone enlongation, or periodontosis in a subject, comprising administering to the subject an effective amount of the isolated polypeptide according to claim 1.

9. A method of treating or preventing osteoarthritis, osteopetrosis, or osteosclerosis, comprising administering to a subject an effective amount of a chemical or neutralizing monoclonal antibodies which inhibit the activity of the polypeptide according to claim 1.

10. A composition comprising an isolated nucleic acid molecule encoding the polypeptide according to claim 1 and a physiologically acceptable excipient.

11. A composition comprising a vector comprising an isolated nucleic acid molecule encoding the polypeptide according to claim 1 and a physiologically acceptable excipient.

12. A method for preventing, treating or controlling osteoporosis, or for fracture healing, bone enlongation or osteopenia, periodontosis, or low bone density or other conditions involving mechanical stress or a lack thereof in a subject, comprising administering to the subject an effective amount of a composition as claimed in claim 11.

13. A method for preventing, treating or controlling osteoporosis, or for fracture healing, bone enlongation or osteopenia, periodontosis, bone fractures or low bone density or other factors causing or contributing to osteoporosis or symptoms thereof or other conditions involving mechanical stress or a lack thereof in a subject, comprising administering the vector as claimed in claim 12 to the subject.

14. A method for preventing, treating or controlling osteoporosis, or for fracture healing, bone enlongation or osteopenia, periodontosis, bone fractures or low bone density or other factors causing or contributing to osteoporosis or symptoms thereof or other conditions involving mechanical stress or a lack thereof in a subject, comprising administering an isolated nucleic acid molecule encoding the polypeptide according to claim 1, or a functional portion thereof or a polypeptide comprising an expression product of the gene or functional portion of the polypeptide or an antibody to the polypeptide or a functional portion of the antibody.
